(19) United States
(12) Patent Application Publication
(10) Pub. No.: US 2021/0338558 A1
(43) Pub. Date: Nov. 4, 2021

(54) USE OF COLLAGEN BINDING DOMAINS TO DELIVER PRODUCTS TO SKIN

(71) Applicant: Fabius Biotechnology, Los Angeles, CA (US)

(72) Inventors: Peter BOASBERG, Los Angeles, CA (US); Marcel E. NIMNI, Santa Monica, CA (US); Bo HAN, Temple City, CA (US)

(21) Appl. No.: 17/280,754

(22) PCT Filed: Sep. 23, 2019
(86) PCT No.: PCT/US2019/052424

§ 371 Date: Mar. 26, 2021

Related U.S. Application Data
(60) Provisional application No. 62/737,579, filed on Sep. 27, 2018.

(51) Int. Cl. A61K 8/65; A61K 8/64; A61K 8/86; A61Q 19/00

(52) U.S. Cl. CPC A61K 8/65; A61K 8/64; A61K 8/86; A61K 2800/654; A61K 2800/412; A61K 2800/57; A61Q 19/00

(57) ABSTRACT

The present invention addresses the need for an improved delivery system that is able to specifically target the skin for improved bioavailability and minimization of side effects resulting from administration of the active ingredient or ingredients. One aspect of the present invention is a targeting composition comprising: (1) a skin care agent or an agent that is a cosmeceutical agent; (2) an intermediate release linker bound to the skin care agent or cosmeceutical agent; (3) a targeting moiety bound to the intermediate release linker, the targeting moiety for binding the targeting composition to native collagen fibers; and (4) optionally, a carrier component to enhance delivery to the skin. The present invention also describes methods of use of these targeting compositions.

### CROSS-REFERENCE TO RELATED APPLICATION

This application claims the benefit of U.S. Provisional Patent Application Ser. No. 62/737,579 by Dr. Peter Boasberg et al., entitled “Use of Collagen Binding Domains to Deliver Products to Skin,” and filed on Sep. 27, 2018, the contents of which are incorporated herein in their entirety by this reference.

### FIELD OF THE INVENTION

This invention is directed to compositions and methods employing collagen binding domains to deliver products to skin.

### BACKGROUND OF THE INVENTION

In many applications, for both cosmetic and therapeutic purposes, which may overlap (hence the term “cosmeceutical”), there is a need for targeting and delivery of cosmetic or therapeutic products to the skin. Although many cosmetic formulas can be directly applied to the skin, in many cases they do not reach their intended biological target and are readily washed away.

The agents for which improved targeting and delivery is required include, but are not limited to, vitamin A and analogs, retinoids, hydroxyacids, cosmeceuticals, skin treatment products, and Wnt pathway modulators.

To understand mechanisms for penetration, the biology of the skin needs to be considered. The main function of the skin is protection, which entails stopping undesirable objects such as bacteria, fungi, viruses, harmful chemicals, or parasites. This barrier function is specific to the superficial part of the skin, the stratum corneum (SC), which has recently been shown to include three sharply demarcated layers with distinct barrier properties. The SC consists of cornified keratinocyte (corneocyte) layers attached to each other by corneodesmosomes, with intercellular spaces sealed by lipids. The intracellular space of corneocytes is filled with keratin filaments, filaggrin and their degradation products.

Each corneocyte is encased in a cornified envelope, which is an insoluble amalgam of proteins highly cross-linked by transglutaminases, the surface of which is tightly bound to intercellular lipids to provide a barrier against the passage of water and water-soluble substances. The SC thus functions as a barrier against foreign insults, as well as a barrier to keep skin hydrated. It is necessary for topical delivery systems to be able to penetrate this to be effective.

Topical delivery systems for cosmetics or cosmeceuticals are not well understood. A topical delivery system or vehicle is defined as the substance that carries a specific active agent into and through the skin. In a broader sense, topical delivery is a strategy to improve the aesthetics and performance of an ingredient or its formulation. The major challenge of topical delivery is the transport of active agents across the skin barrier. However, this is not a straightforward process. Depending on the delivery system, the penetration of the active agent or agents can be variable due to the physicochemical properties of the constituent components of that vehicle.

In general, selection of the appropriate topical delivery system depends on the active agent, anatomical site of application, and consumer preferences for aesthetics and skin feel. One cannot simply add a topical delivery system into an existing formulation and expect it to penetrate. In the selection of a suitable delivery system, all ingredients, including those in the base, must be selected with care for their chemical compatibility, stability and kinetics during skin penetration.

Topical formulations generally have three main constituents: (i) the active agent; (ii) base ingredients including emulsifiers, thickeners, emollients, moisturizers, stabilizers, preservatives, color, fragrance, and other components; and (iii) a medium, i.e., water, anhydrous media, hydroxylic solvents, silicones, or other media. All of these components influence the kinetics of skin penetration.

The skin penetration of salicylic acid offers an example. Salicylic acid is practically insoluble in water at ambient temperatures. It has better solubility in alcohol and other hydroxylic formulation media such as glycols. In a typical skin care lotion or cream that contains about 80% water, the addition of salicylic acid at 2%, for example, even during the manufacturing of the formula at elevated temperatures, may not provide expected levels of efficacy since some or all of it may have crystallized out as fine, imperceptible particles suspended in the matrix of the formulation upon cooling. Such solidified microcrystals would have poor skin penetration. Alternatively, salicylic acid may first be dissolved in alcohol and the resulting solution added to the remaining batch; but again, salicylic acid may separate out as suspended microcrystals as soon as the alcohol dissipates into the water of the base formulation. Clearly, a proper delivery system to prevent such crystallization could provide for more effective delivery of the salicylic acid.

Selecting the correct delivery medium is also of paramount importance. In the case of salicylic acid, it may be dissolved in alcohol or a glycol, i.e., PEG-3 or PEG-4, and the resulting solution may be converted into a gel with a suitable thickening agent. When these are applied topically, two different results may be observed. In the case of alcohol, salicylic acid may appear as a white film on the skin due to the rapid evaporation of alcohol. In the case of a glycol, the formulation would penetrate the skin, leaving no visible residue of salicylic acid. It is worth noting that chemical analyses of the above formulations would indicate nearly the same amount of salicylic acid content, albeit with a different level of skin penetration or bioavailability kinetics, consistent with their consumer-perceived variance of efficacy. As a general rule, an ingredient in a liquid or solution form would have a faster rate of penetration than the same ingredient in a solid or a crystalline state.

Most organic compounds used in topical formulations fall broadly into two classifications, lipophilic or hydrophilic, the incompatibilities of which are bridged by surfactant amphiphilic agents. The lipophilicity or hydrophilicity of such compounds is critical for delivery.

Cosmetic or cosmeceutical ingredients can be formulated for topical delivery through the stratum corneum via a number of general routes.

Many volatile components, such as alcohols such as ethanol or isopropanol, acetone, and silicones, are used in relatively simple cosmetic or cosmeceutical formulations, including gels. However, the use of such volatile components may result in complex formulation effects with respect to delivery or bioavailability. For example, different amounts of the volatile vehicle, such as ethanol, also can influence the skin penetration profile of an active agent. Additionally, caution must be taken with topical formulas based on volatile vehicles that also act as solubilizing mediums for certain ingredients, especially those that have limited solubility or are insoluble in water. Decreased solubility and higher ambient temperatures increase the rate of evaporation of volatile components from the formulation, in turn varying the skin penetration of such ingredients, along with their deposition or crystallization on the skin. Further, when emollients or moisturizers are present in the formula, they can mask the physical appearance of the film deposited on the skin.

As an alternative, non-volatile silicones can be used in topical formulations and have been investigated for their topical delivery efficacy, including, for example, an anhydrous semisolid formulation comprising a novel cross-linked silicone polymer in isododecane solvent. Such an anhydrous semisolid formulation has been studied for delivery of ibuprofen.

As another alternative, liquid crystals such as silicone-based liquid crystals have also been investigated for topical delivery efficacy, including, for example, silicone-based liquid crystals containing polyether functional siloxane as a surfactant, silicon glycol copolymer as an oil phase, and water. Such silicone-based liquid crystals have been studied for their ability to deliver retinyl palmitate.

Retinyl palmitate is frequently incorporated into skin care products and cosmeceuticals. In fact, retinyl palmitate is an example of a prodrug; it is hydrolyzed in vivo to retinol. Other examples of prodrugs are also incorporated into skin care products and cosmeceuticals, such as, but not limited to, derivatives of α-tocopherol (vitamin E) such as tocopheryl acetate or conjugates with unsaturated fatty acids or longer-chain fatty acids, as well as derivatives of ascorbic acid (vitamin C) such as esters including, but not limited to, ascorbyl palmitate. Another approach to prodrug formation used in skin care products and cosmeceuticals are formed via ionic bonding and are sometimes known as ion-pair delivery systems. Examples include complexes of hydroxyacids such as zinc glycinate glycolate and zinc glycinate salicylate.

Another approach to delivery of active agents in cosmetic products and cosmeceuticals is the use of carrier-based, active agent-loaded vesicular and particulate delivery systems. These alternatives include nano- and micro-particle carriers. Commonly used nanocarriers include micelles, liposomes, ethosomes, vesicles, microemulsion polymers, carbon-based materials and other substances that are only a few nanometers in size. Other alternatives include inclusion complexes. Such inclusion complexes employ structured polymers such as cyclodextrin and zeolites. These structured polymers possess openings of defined size to permit the entrance and exit of a host molecule. Still other alternatives include macromolecules such as polyethyleneimine and polyethylene glycol. Still other alternatives include dendrimers. Dendrimers are branched, spherical, three-dimensional polymer structures of specific size, shape and molecular mass with many functional groups; i.e., carboxyl, hydroxyl and amine functional groups. These functional groups form electrostatic or covalent bonds with an active, allowing dendrimers to carry the active inside their structure. Examples of dendrimers include amine-terminated polyamidoamine (PAMAM) dendrimers, peptide dendrimers, and glycopeptide dendrimers. Other carriers include liposomes, niosomes, ethosomes, and discosomes. Liposomes are composed primarily of phospholipids such as phosphatidylcholine, phosphatidylethanolamine, phosphatidylinositol and α-L-dipalmitoyl-phosphatidylcholine that may additionally contain stearylamine, cholesterol or lecithin. Niosomes are two-layered carriers comprised of nonionic surfactants. Ethosomes are vesicular carriers composed of phospholipid, ethanol and water, which are typically used for delivery of lipophilic ingredients. Ethosomes can also be bound to molecular-sized peptides, referred to as skin-penetrating and cell-entering (SPACE) peptides which are conjugated to phospholipids. Discosomes are modified forms of niosomes made from a mixture of ethoxylated cholesterol and ethoxylated fatty alcohols.

Despite the wide range of conventional delivery systems known in the art, there is still a need for an improved delivery system for use in delivering a broad range of active ingredients to the skin for skin care products and cosmeceuticals. In particular, the delivery system should be able to specifically target the skin for improved bioavailability and minimization of side effects resulting from administration of the active ingredient or ingredients.

### SUMMARY OF THE INVENTION

The present invention addresses the need for an improved delivery system that is able to specifically target the skin for improved bioavailability and minimization of side effects resulting from administration of the active ingredient or ingredients.

One aspect of the present invention is a targeting composition comprising:
(1) a skin care agent or an agent that is a cosmeceutical agent;
(2) an intermediate release linker bound to the skin care agent or cosmeceutical agent;
(3) a targeting moiety bound to the intermediate release linker, the targeting moiety for binding the targeting composition to native collagen fibers; and
(4) optionally, a carrier component to enhance delivery to the skin.

The composition can comprise a single skin care agent or an agent that is a cosmeceutical agent bound to an intermediate release linker. Alternatively, the composition comprises two or more different agents, each of which is selected from the group consisting of a skin care agent or a cosmeceutical agent. In another alternative, the composition further comprises at least one additional skin care agent or cosmeceutical that is not bound to the intermediate release linker.

A number of targeting moieties suitable for inclusion in compositions according to the present invention are described. These targeting moieties include peptide sequences present in Von Willebrand's factor, peptide sequences related to peptide sequences present in Von Willebrand's factor by one or more conservative amino acid substitutions, peptide sequences related to peptide sequences present in Von Willebrand's factor by extension at either the amino-terminal or carboxyl-terminal ends, peptide sequences included in elongated structures including multiples of these peptide sequences, collagen binding sites of a platelet collagen binding receptor, such as those selected from the group consisting of integrin α2β1 and glycoprotein VI, and targeting moieties that are collagen-binding domains (CBDs) from discoidin domain receptor DDR1 or DDR2.

The targeting composition can further comprise a cell-penetrating peptide or a transcription-activating peptide.

In one alternative, the intermediate release linker is a polymer that shields the therapeutic agent of the composition from clearance by macrophages. The polymer can be a protein or a non-protein polymer, such as polyethylene glycol. Typically, the intermediate release linker does not interact with the skin care active agent or cosmeceutical and does not bind to or otherwise interact with the targeting moiety.

The linkage between the skin care agent or cosmeceutical and the intermediate release linker is a covalent linkage, such as a cleavable linker; alternatively, the linkage can be a non-covalent linkage such as a biotin/avidin or biotin/streptavidin linkage or a specific antigen/antibody or hapten/antibody linkage. Similarly, the linkage between the intermediate release linker and the targeting moiety can be a covalent linkage, such as a cleavable linker; alternatively, the linkage can be a non-covalent linkage such as a biotin/avidin or biotin/streptavidin linkage or a specific antigen/antibody or hapten/antibody linkage.

Typically, the skin care agent is selected from the group consisting of retinoids, hydroxyacids, esters of hydroxyacids, skin treatment products, and Wnt pathway modulators.

Typically, the cosmeceutical is selected from the group consisting of a botanical extract from oil palm vegetation liquor; GM-CSF; a nucleic acid expressing GM-CSF; a suspension of a powder of an aliphatic polyester copolymer, a cross-linked silicone elastomer, and at least one hydrolysate or acylated short-chain peptide; a mixture of refined, bleached, deodorized palm oils and red palm olein; a dipeptide incorporating a selenoamino acid; a 3,6-dihydro-2H-pyran; calcium chloride, magnesium chloride, and potassium bromide for restoration of skin barrier function; a composition including nordihydroguiaretic acid, niacinimide, and, optionally, an antioxidant; a peptide modified with a triterpenoid; 5-aminolevulinic acid; 3,5-dimethoxy-4′-hydroxystilbene; an alkanediol selected from the group consisting of 1,2-propanediol, butyleneglycol, 2-ethyl-1,3-hexanediol, and 2-methyl-2,4-pentanediol; an ether diol; a diether alcohol; a composition including hyaluronic acid, kokic acid, and glycolic acid; artemetin; hydroquinone or a derivative thereof; an anti-acne agent selected from the group consisting of N-acetylcysteine, adapalene, azelaic acid, benzoyl peroxide, cholate, clindamycin, deoxycholate, erythromycin, flavonoids, glycolic acid, meclocycline, mupirocin, octopirox, phenoxyethanol, phenoxypropanol, pyruvic acid, resorcinol, retinoic acid, salicylic acid, scymnol sulfate, sulfacetamide-sulfur, sulfur, tazarotene, tetracycline, and tretinoin triclosan; melatonin; an anti-psoriatic agent selected from the group consisting of 6-aminonicotinam ide, 6-aminonicotinic acid, 2-aminopyrazinamide, anthralin, calcipotriene, 6-carbamoylnicotinamide, 6-chloronicotinamide, 2-carbamoylpyrazinam ide, corticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, and trichothecene mycotoxins; an anti-rosacea agent selected from the group consisting of azelaic acid and metronidazole sulfacetamide; a histamine receptor H₁ antagonist selected from the group consisting of doxepin hydrochloride, carbinoxamine maleate, clemastine fumarate, diphenhydramine hydrochloride, dimenhydrinate, pyrilamine citrate, tripelennamine hydrochloride, tripelennamine citrate, chlorpheniramine mdialeate, brompheniramine maleate, hydroxyzine hydrochloride, hydroxyzine pamoate, cyclizine hydrochloride, cyclizine lactate, meclizine hydrochloride, promethazine hydrochloride, cyproheptadine hydrochloride, phenindamine tartrate, acrivastine, cetirizine hydrochloride, azelastine hydrochloride, levocabastine hydrochloride, loratidine, desloratidine, ebastine, mizolastine, and fexofenadine; a histamine receptor H₂ antagonist selected from the group consisting of cimetidine and ranitidine; a histamine receptor H₃ antagonist; a histamine receptor H₄ antagonist; a kinin receptor antagonist; a leukotriene receptor antagonist; vitamin E; vitamin E acetate; tocotrienol; progesterone; capsaicin; capsicum oleoresin; menthol; methyl salicylate; benzophenone-3; octyl methoxycinnamate; benzocaine; and lidocaine.

Typically, if present, the optional carrier component is a pharmaceutically acceptable carrier, diluent, or excipient. Preferably, the pharmaceutically acceptable carrier, diluent, or excipient is a dermatologically acceptable carrier, diluent, or excipient. Various components can be included in the optional carrier component.

Another aspect of the present invention is a method of treating a subject with a skin care agent or cosmeceutical as described above to effect an esthetic improvement in the subject. The method comprises administering a therapeutically effective quantity of a targeting composition according to the present invention to effect an esthetic improvement in the subject. The esthetic improvement can be, but is not limited to, selected from the group consisting of removal or reduction of blemishes, removal or reduction of wrinkles, and removal or reduction of irregularities in skin color or skin tone. Typically, the targeting composition is administered topically. Typically, the targeting composition includes the optional carrier component. In one alternative, the targeting composition is administered in a pharmaceutical composition including at least one additional skin care agent or cosmeceutical.

### BRIEF DESCRIPTION OF THE DRAWINGS

FIG. 1 shows a native collagen fiber stained with phosphotungstic acid, showing 68-nm periodicity and a schematic representation of collagen molecules measuring approximately 300 nm (adapted from M. Nimni, ed., “Collagen”, Vol. 1, CRC Press, 1988).

FIG. 2 shows the molecular packing of the Type I collagen fiber.

FIG. 3 depicts a genetically engineered fusion protein consisting of TGF-β1 with a collagen binding decapeptide. The purification tag comprises a hexapeptide of histidine, linked via a Gly-Gly link; it binds tightly to a Ni-NTA column. DNA constructs were transfected into Escherichia coli.

FIG. 4 depicts the binding of the TGF-β with a collagen binding domain to collagen; the binding requires a high concentration of urea for dissociation. This is compared to the behavior of TGF-β without the collagen binding domain, which has poor affinity for collagen.

FIG. 5 shows molecular modeling of discoidin, including the amino acids on the surface involved in binding to collagen. These amino acids and their distribution within the DS domain provide a three-dimensional view of the nature of the collagen-ligand interaction.

FIG. 6 is a schematic drawing of molecular packing within a collagen fiber. (A) Axial view showing linear staggering; (B) Cross-sectional view showing the unit cell. (B) shows how particular segments are repeated on the surface of the fiber (b-b for instance is separated by 2× the diameter of a molecule or approximately 3 nm laterally, the distance that repeating CBDs should be set apart for optimal binding).

FIG. 7 is a diagrammatic representation of a collagen targeting vector: (A) CBD; (B) peptide for facilitating skin care agent or cosmeceutical (D) attachment (length of peptide and specific amino acids in peptide leading to suitable conformations in solution will vary); (C) reactive functional groups suitable for drug attachment (—SH, —CO₂H, —NH₂, or other groups); (D) skin care agent or cosmeceutical; (E) additional site for identical or different CBD, separated by a suitable length of spacer (B) can be added.

FIG. 8 shows the entire wild-type DDR2 amino acid sequence.

### DETAILED DESCRIPTION OF THE INVENTION

Accordingly, one aspect of the present invention is a targeting composition comprising:

(1) a skin care agent or an agent that is a cosmeceutical agent;

(2) an intermediate release linker bound to the skin care agent or cosmeceutical agent;

(3) a targeting moiety bound to the intermediate release linker, the targeting moiety for binding the targeting composition to native collagen fibers; and

(4) optionally, a carrier component to enhance delivery to the skin.

In one alternative, the composition comprises a single skin care agent or an agent that is a cosmeceutical agent bound to an intermediate release linker. In another alternative, the composition comprises two or more different agents, each of which is selected from the group consisting of a skin care agent and a cosmeceutical agent.

The composition can additionally comprise at least one additional skin care agent or cosmeceutical that is not bound to the intermediate release linker.

The peptide motif can be selected from the group consisting of: Trp-Arg-Glu-Pro-Ser-Phe-Met-Ala-Leu-Ser (WREPSFMALS) (SEQ ID NO: 1); Trp-Arg-Glu-Pro-Ser-Phe-Cys-Ala-Leu-Ser (WREPSFCALS) (SEQ ID NO: 2); and peptides related to SEQ ID NO: 1 or SEQ ID NO: 2 by one or more conservative amino acid substitutions.

Alternatively, the peptide motif can be a peptide related to SEQ ID NO: 1 or SEQ ID NO: 2 by one or more conservative amino acid substitutions, wherein the peptide is selected from the group consisting of: Trp-Arg-Asp-Pro-Ser-Phe-Met-Ala-Leu-Ser (WREPSFMALS) (SEQ ID NO: 3); Trp-Arg-Asp-Pro-Ser-Phe-Cys-Ala-Leu-Ser (WRDPSFCALS) (SEQ ID NO: 4); Trp-Arg-Glu-Pro-Ser-Phe-Met-Ala-Ile-Ser (WREPSFMAIS) (SEQ ID NO: 5); Trp-Arg-Glu-Pro-Ser-Phe-Cys-Ala-Ile-Ser (WREPSFCAIS) (SEQ ID NO: 6); Trp-Arg-Asp-Pro-Ser-Phe-Met-Ala-Ile-Ser (WRDPSFMAIS) (SEQ ID NO: 7); and Trp-Arg-Asp-Pro-Ser-Phe-Cys-Ala-Ile-Ser (WRDPSFCAIS) (SEQ ID NO: 8).

In still another alternative, the peptide motif can be a peptide selected from the group consisting of: Gly-Pro-Pro-Gly-Trp-Arg-Glu-Pro-Ser-Phe-Met-Ala-Leu-Ser-Gly-Pro-Pro-Gly (GPPGWREPSFMALSGPPG) (SEQ ID NO: 9); Gly-Pro-Pro-Gly-Trp-Arg-Glu-Pro-Ser-Phe-Cys-Ala-Leu-Ser-Gly-Pro-Pro-Gly (GPPGWREPSFCALSGPPG) (SEQ ID NO: 10); Gly-Pro-Pro-Gly-Trp-Arg-Asp-Pro-Ser-Phe-Met-Ala-Leu-Ser-Gly-Pro-Pro-Gly (GPPGWRDPSFMALSGPPG) (SEQ ID NO: 11); Gly-Pro-Pro-Gly-Trp-Arg-Asp-Pro-Ser-Phe-Cys-Ala-Leu-Ser-Gly-Pro-Pro-Gly (GPPGWRDPSFCALSGPPG) (SEQ ID NO: 12); Gly-Pro-Pro-Gly-Trp-Arg-Glu-Pro-Ser-Phe-Met-Ala-Ile-Ser-Gly-Pro-Pro-Gly (GPPGWREPSFMAISGPPG) (SEQ ID NO: 13); Gly-Pro-Pro-Gly-Trp-Arg-Glu-Pro-Ser-Phe-Cys-Ala-Ile-Ser-Gly-Pro-Pro-Gly (GPPGWREPSFCAISGPPG) (SEQ ID NO: 14); Gly-Pro-Pro-Gly-Trp-Arg-Asp-Pro-Ser-Phe-Met-Ala-Ile-Ser-Gly-Pro-Pro-Gly (GPPGWRDPSFMAISGPPG) (SEQ ID NO: 15); and Gly-Pro-Pro-Gly-Trp-Arg-Asp-Pro-Ser-Phe-Cys-Ala-Ile-Ser-Gly-Pro-Pro-Gly (GPPGWRDPSFCAISGPPG) (SEQ ID NO: 16).

In still another alternative, the peptide motif is an elongated peptide structure of Formula (I):

(I)
[Gly-Pro-Pro-Gly-X₁-Gly-Pro-Pro-Gly-X₂-Gly-Pro-
Pro-Gly]ₙ

wherein: (1) X₁ and X₂ are one of peptide sequences SEQ ID NO: 1 through SEQ ID NO: 16; and (2) n is an integer from 1 to 15.

In yet another alternative, the targeting moiety can be a collagen binding site of a platelet collagen binding receptor, including, but not limited to, integrin α2β1 and glycoprotein VI.

In still another alternative, the targeting moiety can be a targeting moiety in which the peptide sequence WREPSFMALS (SEQ ID NO: 1) or WREPSFCALS (SEQ ID NO: 2) is incorporated into a molecule to generate a peptide of from about 2,000 daltons to about 10,000 daltons in molecular weight. In this alternative, the flanking sequences can mimic a sequence found in native collagen or native elastin; the targeting moiety can also include at least one reactive amino acid. The targeting moiety can include two or three collagen binding domains, with the collagen binding domains being separated by spacers. The spacers can provide laterally displaced equivalent sites with a lateral displacement of about 3 nm. The spacers can elongate in solution. The spacers can include alternating polar and nonpolar sequences; alternatively, the spacers can include polylysine or polyglycine residues.

The targeting moiety can be pegylated.

The targeting moiety can include a peptide sequence including an amino-terminal amino acid that is acetylated, or can include a peptide sequence including a carboxyl-terminal amino acid that is amidated. The targeting moiety can include a fluorescein moiety for labeling.

In yet another alternative, the targeting moiety includes the amino acid sequence GVMGFO (SEQ ID NO. 17).

In still another alternative, the targeting moiety includes a collagen-binding domain (CBD) from discoidin domain receptor DDR1 or DDR2, or includes a CBD incorporating the amino acids on the surface of the three-dimensional protein structure of DDR1 or DDR2 in which at least one of the amino acids not directly contacting collagen is replaced with a conservative amino acid substitution such that the CBD binds collagen with a binding affinity of at least 80% of the binding affinity of the native CBD from DDR1 or DDR2.

In still another alternative, the targeting moiety includes the amino acid sequence GTPGPGGIAGQRGVV (SEQ ID NO: 19), or includes an amino acid sequence derived from GTPGPGGIAGQRGVV (SEQ ID NO: 19) by one or more conservative amino acid substitutions such that the CBD binds collagen with a binding affinity of at least 80% of the binding affinity of the sequence GTPGPGGIAGQRGVV (SEQ ID NO: 19).

In one alternative, the skin care agent or cosmeceutical includes one or more optional substitutions in an organic molecule or organic portion of a molecule as described below. Such optional substitutions can be introduced either to facilitate the linkage of the skin care agent or cosmeceutical agent to the intermediate release linker or to improve the activity, specificity, or stability of the skin care agent or cosmeceutical.

When the skin care agent or cosmeceutical includes multiple components, one or more of the components may be covalently linked to the intermediate release linker. Alternatively, one or more of the components may be non-covalently linked to the intermediate release linker through such interactions as hydrophobic interactions, hydrogen bonds, or salt links as is understood in the art. If one or more of the components of the skin care agent or cosmeceutical is an inorganic molecule, structure, or moiety, the inorganic molecule or moiety can be linked to the intermediate release linker through suitable interactions such as ionic interactions. For skin care agents or cosmeceuticals having multiple components, one or more of the components may be covalently linked to the intermediate release linker. In some alternatives, the component that is covalently or non-covalently linked to the intermediate release linker may be further covalently or non-covalently linked to another component of the skin care agent or cosmeceutical.

In one alternative, the skin care agent is selected from the group consisting of: retinoids, including but not limited to vitamin A and analogs thereof; hydroxyacids; esters of hydroxyacids; cosmeceuticals; skin treatment products; and Wnt pathway modulators.

Retinoids suitable for use in methods and compositions according to the present invention include, but are not limited to the retinoids recited below.

Suitable retinoids include, but are not limited to, retinol, retinal, tretinoin (retinoic acid), isotretinoin, alitretinoin, etretinate, acitretin, adapalene, bexarotene, and tazarotene.

Additional retinoids include the following:

U.S. Pat. No. 9,855,244 to Duprat et al. discloses 3″-t-butyl-4′-(2-hydroxyethoxy)-4″-pyrrolidin-1-yl[1,1′;3′,1″]terphenyl-4-carboxylic acid as well as compounds of Formula (R-I):

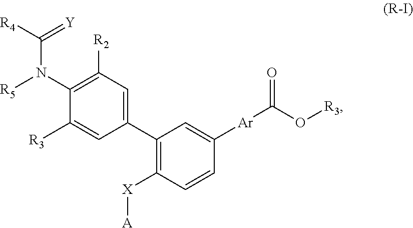

wherein:

(1) R₁ is hydrogen, C₁-C₄ alkyl, or —CF₃;

(2) R₂ is hydrogen, C₁-C₄ alkyl, C₁-C₄ alkoxy, or chloro;

(3) R₃ is hydrogen, a linear or branched C₁-C₁₀ alkyl or alkoxy group optionally substituted with a methoxy group, or a linear or branched C₁-C₁₀ alkyl group containing an ether function;

(4) R₄ is hydrogen or C₁-C₃ alkyl;

(5) R₅ is hydrogen or C₁-C₃ alkyl; or, alternatively, R₄ and R₅, together with a —N—C(═Y)— moiety, a pyrrolidine, pyrrolidinone, piperidine or piperidinone ring;

(6) Y is two hydrogen atoms or a heteroatom such as oxygen or sulfur;

(7) Ar is a 1,4-phenyl, 2,5-pyridyl, 5,2-pyridyl or 2,5-thiophenyl ring;

(8) X is an oxygen atom optionally substituted with an alkyl or alkylamine chain or a C— single bond;

(9) A is a hydrogen atom or a moiety of Formula (R-I(a)):

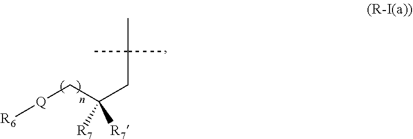

wherein: (a) Q is an oxygen atom or an —NH— bond; (b) R₆ is hydrogen, C₁-C₆ alkyl, C₃-C₆ cycloalkyl, —C(O)CH₃, or —C(O)CH₂CH₃; (c) R₇ and R_{7′} are hydrogen or hydroxyl, with the proviso that R₇ and R_{7′} are not both hydroxyl; and n is 0, 1, 2, 3, 4, or 5.

U.S. Pat. No. 9,549,884 to Gaboardi et al. discloses retinoids including 2-(nicotinamido)-ethyl retinoate, 2-(nicotinamido)-butyl retinoate, 5-(nicotinamido)-pentyl retinoate, 2-(nicotinamido)-hexyl retinoate, and retinyl retinoate.

U.S. Pat. No. 9,456,984 to Niitsu et al. discloses retinoids including retinyl palmitate and fenretinide.

U.S. Pat. No. 9,399,023 to Ditzinger et al. discloses retinoids including 9-cis-retinal and 9-cis-retinol.

U.S. Pat. No. 9,339,509 to Claggett-Dame et al. discloses retinoids including tamibarotene, retinoyl t-butyrate, retinoyl pinacol and retinoyl cholesterol.

U.S. Pat. No. 9,193,672 to Yu discloses retiferol and prodrugs of retinoids.

U.S. Pat. No. 8,962,691 to Palczewski et al. discloses synthetic retinoids including 9-ethyl-11-cis-retinal, 7-methyl-1-cis-retinal, 13-desmethyl-11-cis-retinal, 11-cis-10-F-retinal, 11-cis-10-Cl-retinal, 11-cis-10-methyl-retinal, 11-cis-10-ethyl-retinal, 9-cis-10-F-retinal, 9-cis-10-Cl-retinal, 9-cis-10-methyl-retinal, 9-cis-10-ethyl-retinal, 11-cis-12-F-retinal, 11-cis-12-Cl-retinal, 11-cis-12-methyl-retinal, 11-cis-10-ethyl-retinal, 9-cis-12-F-retinal, 9-cis-12-Cl-retinal, 9-cis-12-methyl-retinal 11-cis-14-F-retinal, 11-cis-14-methyl-retinal, 11-cis-14-ethyl-retinal, 9-cis-14-F-retinal, 9-cis-14-methyl-retinal, and 9-cis-14-ethyl-retinal, as well as derivatives having a modified ring structure.

U.S. Pat. No. 8,633,335 to Muratake et al. discloses retinoid prodrugs including compounds of Formula (R-II)):

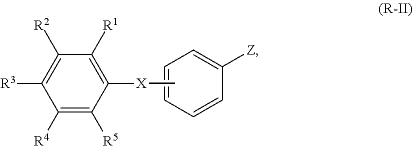

wherein:

(1) R¹, R², R³, R⁴, and R⁵ are each hydrogen, lower alkyl, or tri(lower alkyl) silyl where two adjacent lower alkyl groups represented by them may bond together to form a 5- or 6-membered ring which may have one or two or more alkyl groups together with the carbon atoms in the benzene ring to which they bond;

(2) X is —NH—CO—, —CO—NH—, —N(COR⁶)—CO—, —CO—N[CON(R⁸)(R⁹)]—, or —N[CON(R¹⁰)(R¹¹)];

(3) R⁶ and R⁷ are an optionally substituted lower alkoxy group or a phenyl group has at least one alkoxycarbonyl group or carboxy group as a substituent and optionally has another substituent;

(4) R⁸, R⁹, R¹⁰, and R¹¹ are hydrogen or lower alkyl;

(5) Z is Y—CH(R¹²)—COOH, —CHO, —CH═CH—COOH, or —COOR¹³;

(6) Y is a single valence bond, CH₂—, —CH(OH)—, —CO—, —CO—NH—, or —CO—NH—CH₂—CO—NH—;

(7) R¹² is hydrogen or lower alkyl;

(8) R¹³ is hydrogen, —CH(R¹⁴)—, —[CH₂CH₂—O]ₙ—CH₂—CH₂—OH, —CH₂—O—[CH₂CH₂—O]ₘ—CH₂—OH, or —[CH(CH₃)—CO—O]ₚ—CH(CH₃)—COOH;

(9) R¹⁴ is hydrogen, lower alkyl, or hydroxy;

(10) m is 1-100;

(11) n is 1-100;

(12) p is 1-100; and

(13) with the proviso that when X is —NH—CO— or —CO—NH—, R¹³ is other than hydrogen.

U.S. Pat. No. 8,530,517 to Cabri et al. discloses synthetic retinoids, including (S)-2-amino-3-methyl-butyric acid 3-adamantan-1-yl-4′-((E)-2-carboxy-vinyl)-biphenyl-4-yl ester hydrochloride; (E)-3-(3′-adamantan-1-yl-4′-{2-[2-(2-carboxymethoxy-ethoxy)-ethoxy]-acetoxy}-biphenyl-4-yl)-acrylic acid; undecanoic acid 3-adamantan-1-yl-4′-(E)-2-carboxy-vinyl)-biphenyl-4-yl ester; 4-morpholin-4-yl-butyric acid 3-adamantan-1-yl-4′-((E)-2-carboxy-vinyl)-biphenyl-4-yl ester hydrochloride; 4-(4-methyl-piperazin-1-yl)-butyric acid 3-adamantan-1-yl-4′-((E)-2-carboxy-vinyl)-biphenyl-4-yl ester dihydrochloride; (E)-3-[3′-adamantan-1-yl-4′-(2-methylamino-ethylcarbamoyloxy)-biphenyl-4-yl]-acrylic acid; (E)-3-(3′-adamantan-1-yl-4′-carboxymethylcarbamoyloxy-biphenyl-4-yl)-acrylic acid; (E)-3-[3′-adamantan-1-yl-4′-(4-amino-butylcarbamoyloxy)-biphenyl-4-yl]-acrylic acid hydrochloride; (E)-3-[3′-adamantan-1-yl-4′-(2-morpholin-4-yl-ethyl-carbamoyloxy)-biphenyl-4-yl]-acrylic acid hydrochloride; (E)-3-(3′-adamantan-1-yl-4′-undecyl-carbamoyloxy-biphenyl-4-yl)-acrylic acid; [1,4′]bipiperidinyl-1′-carboxylic acid 3-adamantan-1-yl-4′-((E)-2-carboxy-vinyl)-biphenyl-4-yl ester hydrochloride; (E)-3-(3′-adamantan-1-yl-4′-isopropylcarbamoyloxy-biphenyl-4-yl)-acrylic acid; 4-[3-adamantan-1-yl-4′-((E)-2-carboxy-vinyl)-biphenyl-4-yloxycarbonylamino]-piperidine-1-carboxylic acid benzyl ester; (E)-3-{3′-adamantan-1-yl-4′-[(S)-1-(carboxymethyl-carbamoyl)-2-methyl-propylcarbamoyloxy]-biphenyl-4-yl}-acrylic acid; (E)-3-[3-adamantan-1-yl-4′-(2-methoxy-ethoxymethoxy)-biphenyl-4-yl]-acrylic acid; cyclopropanecarboxylic acid 3-adamantan-1-yl-4′-((E)-2-carboxy-vinyl)-biphenyl-4-yl ester; E)-3-[3′-adamantan-1-yl-4′-(1,3-dioxo-1,3-dihydro-isoindol-2-ylmethoxy)-biphenyl-4-yl]-acrylic acid; (9Z,12E)-octadeca-9,12-dienoic acid 3-adamantan-1-yl-4′-((E)-2-carboxy-vinyl)-biphenyl-4-yl ester; (E)-3-(3′-adamantan-1-yl-4′-propoxycarbonyloxymethoxy-biphenyl-4-yl)-acrylic acid; 1-amino-cyclopropanecarboxylic acid 3-adamantan-1-yl-4′-((E)-2-carboxy-vinyl)-biphenyl-4-yl ester; (E)-3-(3′-adamantan-1-yl-4′-cyanomethoxy-biphenyl-4-yl)-acrylic acid; (E)-3-(3′-adamantan-1-yl-4′-carbamoylmethoxy-biphenyl-4-yl)-acrylic acid; and (E)-3-[3′-adamantan-1-yl-4′-(2-morpholin-4-yl-ethoxy)-biphenyl-4-yl]-acrylic acid.

U.S. Pat. No. 8,529,920 to Liu et al. discloses retinoids including retinyl acetate, retinyl propionate, dehydroretinol, eretinate, eretrin, and motretinide.

U.S. Pat. No. 8,293,803 to Przyborski discloses synthetic retinoids including compounds of Formula (R-III):

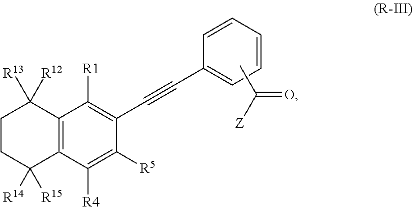

wherein:

(1) R¹, R⁴ and R₅ are each independently selected from hydrogen, R⁶, hydrocarbyl optionally substituted with 1, 2, 3, 4 or 5 R⁶, and —(CH₂)ₖ-heterocyclyl optionally substituted with 1, 2, 3, 4 or 5 R⁶;

(2) each R⁶ is independently selected from halogen, trifluoromethyl, cyano, nitro, oxo, ═NR⁷, —OR⁷, —C(O)R⁷, —C(O)OR⁷, —OC(O)R⁷, —S(O)/R⁷, —N(R⁷)R⁸, —C(O)N(R⁷)R⁸, —S(O)N(R⁷)R⁸ and R⁹;

(3) R⁷ and R⁸ are each independently hydrogen or R⁹;

(4) R⁹ is selected from hydrocarbyl and —(CH₂)k-heterocyclyl, either of which is optionally substituted with 1, 2, 3, 4 or 5 substituents independently selected from halogen, cyano, amino, hydroxy, C₁-C₆ alkyl and C₁-C₆ alkoxy;

(5) k is 0, 1, 2, 3, 4, 5 or 6;

(6) l is 0, 1 or 2;

(7) R¹¹, R¹², R¹⁴ and R¹⁵ are each independently selected from hydrogen, R⁶, hydrocarbyl optionally substituted with 1, 2, 3, 4 or 5 R⁶, and —(CH₂)ₖ-heterocyclyl optionally substituted with 1, 2, 3, 4 or 5 R⁶; and

(8) Z is selected from —OH and NHOH; and

wherein any one or more of the aliphatic and the aromatic groups of Formula (R-III) may optionally be substituted with one or more R⁶ groups.

U.S. Pat. No. 8,110,703 to Chen et al. discloses synthetic retinoids, including (4-trifluoromethylphenyl)-(all-trans)-3,7-dimethyl-9-(2,6,6-trimethyl-1-cyclohexen-1-yl)-2,4,6,8-nonatetraenoate; (3-trifluoromethylphenyl)-(all-trans)-3,7-dimethyl-9-(2,6,6-trimethyl-1-cyclohexen-1-yl)-2,4,6,8-nonatetraenoate; (2-trifluoromethylphenyl)-(all-trans)-3,7-dimethyl-9-(2,6,6-trimethyl-1-cyclohexen-1-yl)-2,4,6,8-nonatetraenoate; (4-trifluoromethylphenyl)-(all-trans)-3,7-dimethyl-9-(2,6,6-trimethyl-1-cyclohexen-1-yl)-2,4,6,8-nonatetraenoic acid amide; (3-trifluoromethylphenyl)-(all-trans)-3,7-dimethyl-9-(2,6,6-trimethyl-1-cyclohexen-1-yl)-2,4,6,8-nonatetraenoic acid amide; (4-amino-3-trifluoromethylphenyl)-(all-trans)-3,7-dimethyl-9-(2,6,6-trimethyl-1-cyclohexen-1-yl)-2,4,6,8-nonatetraenoate; (4-amino-2-trifluoromethylphenyl)-(all-trans)-3,7-dimethyl-9-(2,6,6-trimethyl-1-cyclohexen-1-yl)-2,4,6,8-nonatetraenoate; (4-trifluoromethoxyphenyl)-(all-trans)-3,7-dimethyl-9-(2,6,6-trimethyl-1-cyclohexen-1-yl)-2,4,6,8-nonatetraenoate; (3-trifluoromethoxyphenyl)-(all-trans)-3,7-dimethyl-9-(2,6,6-trimethyl-1-cyclohexen-1-yl)-2,4,6,8-nonatetraenoate; (4-trifluoromethoxyphenyl)-(all-trans)-3,7-dimethyl-9-(2,6,6-trimethyl-1-cyclohexen-1-yl)-2,4,6,8-nonatetraenoic acid amide; (3-trifluoromethoxyphenyl)-(all-trans)-3,7-dimethyl-9-(2,6,6-trimethyl-1-cyclohexen-1-yl)-2,4,6,8-nonatetraenoic acid amide; (4-hydroxy-3-trifluoromethylphenyl)-(all-trans)-3,7-dimethyl-9-(2,6,6-trimethyl-1-cyclohexen-1-yl)-2,4,6,8-nonatetraenoic acid amide; (4-hydroxy-2-trifluoromethylphenyl)-(all-trans)-3,7-dimethyl-9-(2,6,6-trimethyl-1-cyclohexen-1-yl)-2,4,6,8-nonatetraenoic acid amide; (3-trifluoromethylphenyl)-(all-trans)-9-(4-methoxy-2,3,6-trimethylphenyl)-3,7-dimethyl-2,4,6,8-nonatetraenoate; (2-trifluoromethylphenyl)-(all-trans)-9-(4-methoxy-2,3,6-trimethylphenyl)-3,7-dimethyl-2,4,6,8-nonatetraenoate; (4-trifluoromethylphenyl)-(all-trans)-9-(4-methoxy-2,3,6-trimethylphenyl)-3,7-dimethyl-2,4,6,8-nonatetraenoate; (3-trifluoromethoxyphenyl)-(all-trans)-9-(4-methoxy-2,3,6-trimethylphenyl)-3,7-dimethyl-2,4,6,8-nonatetraenoic acid amide; (2-trifluoromethoxyphenyl)-(all-trans)-9-(4-methoxy-2,3,6-trimethylphenyl)-3,7-dimethyl-2,4,6,8-nonatetraenoic acid amide; (4-amino-3-trifluoromethylphenyl)-(all-trans)-9-(4-methoxy-2,3,6-trimethylphenyl)-3,7-dimethyl-2,4,6,8-nonatetraenoate; (4-amino-2-trifluoromethylphenyl)-(all-trans)-9-(4-methoxy-2,3,6-trimethylphenyl)-3,7-dimethyl-2,4,6,8-nonatetraenoate; (4-trifluoromethoxyphenyl)-(all-trans)-9-(4-methoxy-2,3,6-trimethylphenyl)-3,7-dimethyl-2,4,6,8-nonatetraenoate; (3-trifluoromethoxyphenyl)-(all-trans)-9-(4-methoxy-2,3,6-trimethylphenyl)-3,7-dimethyl-2,4,6,8-nonatetraenoate; (3-trifluoromethoxyphenyl)-(all-trans)-9-(4-methoxy-2,3,6-trimethylphenyl)-3,7-dimethyl-2,4,6,8-nonatetraenoic acid amide; (2-trifluoromethoxyphenyl)-(all-trans)-9-(4-methoxy-2,3,6-trimethylphenyl)-3,7-dimethyl-2,4,6,8-nonatetraenoic acid amide; (4-trifluoromethoxyphenyl)-(all-trans)-9-(4-methoxy-2,3,6-trimethylphenyl)-3,7-dimethyl-2,4,6,8-nonatetraenoic acid amide; (4-hydroxy-3-trifluoromethylphenyl)-(all-trans)-9-(4-methoxy-2,3,6-trimethylphenyl)-3,7-dimethyl-2,4,6,8-nonatetraenoic acid amide; or (4-hydroxy-2-trifluoromethylphenyl)-(all-trans)-9-(4-methoxy-2,3,6-trimethylphenyl)-3,7-dimethyl-2,4,6,8-nonatetraenoic acid amide.

U.S. Pat. No. 8,101,793 to Merlini et al. discloses retinoid derivatives that are adamantyl derivatives with two phenyl moieties.

U.S. Pat. No. 7,964,639 to DeLuca et al. discloses retinoid esters with sterically hindered alcohols such as secondary or tertiary alcohols.

U.S. Pat. No. 7,166,744 to Sin et al. discloses retinoids including 2-butyryloxy-5-(2E,4E,6E,8E)-[3,7-dimethyl-9-(2,6,6-trimethyl-cyclohex-1-enyl)-nona-2,4,6,8-tetraenoylamino]-phenyl butanoate; 5-(2E,4E,6E,8E)-[3,7-dimethyl-9-(2,6,6-trimethyl-cyclohex-1-enyl)-nona-2,4,6,8-tetraenoylamino]-2-hydroxy-phenyl butanoate; and (2E,4E,6E,8E)-[3,7-dimethyl-9-(2,6,6-trimethyl-cyclohex-1-enyl)-nona-2,4,6,8-tetraenoylamino]-(3-butylamino-4-hydroxy)-phenylamide.

U.S. Pat. No. 7,074,420 to Duggan et al. discloses retinoxytrimethylsilane.

U.S. Pat. No. 6,858,647 to Voegel et al. discloses retinoids including 6-[3-(1-adamantyl)-4-methoxy-5-hydroxyphenyl]-2-naphthoic acid; 4-[4-(6-methoxymethoxy-4′-methylbiphenyl-2-yl)but-3-en-1-ynyl]benzoic acid; 4-[4-(6-methoxy-4′-methylbiphenyl-2-yl)but-3-en-1-ynyl]benzoic acid; 6-[2-methyl-4-hydroxy-5-(1-adamantyl)phenyl]-2-naphthoic acid; 4-[3-(3,5-di-t-butyl-4-oxocyclohexa-2,5-dienylidene)prop-1-ynyl]benzoic acid; 2-hydroxy-4-[4-(4′-methylbiphenyl-2-yl)but-3-en-1-ynyl]benzoic acid; 4-[4-(4,4′-dimethylbiphenyl-2-yl)but-3-en-1-ynyl]benzoic acid; 6-(3-adamantan-1-yl-5-bromo-4-hydroxyphenyl)naphthalene-2-carboxylic acid; 4-[4-(6,4′-dimethylbiphenyl-2-yl)but-3-en-1-ynyl]benzoic acid; 4-[4-(4′-propylbiphenyl-2-yl)but-3-en-1-ynyl]benzoic acid; (E)-4-[4-(5-methoxymethoxy-4′-methylbiphenyl-2-yl)but-3-en-1-ynyl]benzoic acid; 4-[4-(3-methoxy-4′-methylbiphenyl-2-yl)but-3-en-1-ynyl]benzoic acid; and 4-[4-(4′-methylbiphenyl-2-yl)but-3-en-1-ynyl]benzoic acid.

U.S. Pat. No. 6,777,418 to Lapierre et al. discloses retinoid derivatives including a 1,1,4,4-tetramethyl-1,2,3,4-tetrahydronaphthalenyl moiety.

U.S. Pat. No. 6,759,396 to Michel et al. discloses 6-[3-(1-adamantyl)-4-hydroxyphenyl]-2-naphthoic acid and 6-[3-(1-adamantyl)-4-methoxyphenyl]-2-naphthoic acid.

U.S. Pat. No. 6,603,012 to Belloni et al. discloses tetrahydronaphthalen-2-yl)-alkyloxy]-benzoic acid derivatives as retinoids.

U.S. Pat. No. 6,083,977 to Boehm et al. discloses trienoic retinoids.

U.S. Pat. No. 5,863,942 to Duffy et al. discloses conjugates of retinoids and a bioactive organic acid preferably selected from among alpha-hydroxy acids, beta-hydroxy acids, and keto-acids; preferred conjugates include retinyl glycolyl ether and retinyl glycolate (as either the ester or reverse ester).

U.S. Pat. No. 5,798,372 Davies et al. discloses retinoids including (E)-3,7-dimethyl-9-(2,6,6-trimethyl-1-cyclohexen-1-yl)-2,4,6,8-nonatetraenoic acid; (2Z,4E,6E,8E)-3, 7-dimethyl-9-(2,6,6-trimethyl-1-cyclohexen-1-yl)-2,4,6,8-nonatetraenoic acid; (E)-4-[4-(2,6,6-trimethyl-1-cyclohexen-1-yl) but-2-en-1-ynyl] benzoic acid; 4-[2-(5,6,7,8-tetrahydro-5,5,8,8-tetramethyl-2-naphthalenyl)ethynyl] benzoic acid; (E)-4-[2-(5,6,7,8-tetrahydro-5,5,8,8-tetramethyl-2-naphthalenyl)-1-propen-1-yl] benzoic acid; 4-[2-(3-(2-tetrahydropyranyl)oxy)-(4-(1,1-dimethylethyl)phenyl)ethynyl]benzoic acid; 6-[2-(3-(2-tetrahydropyranyl)oxy)-(4-(1,1-dimethylethyl)phenyl)ethynyl]-3-nicotinic acid; (2E,4E,6Z,8E)-3,7-dimethyl-9-(2,6,6-trimethyl-1-cyclohexen-1-yl)-2,4,6,8-nonatetraenoic acid; and (E)-5-[2-(5,6,7,8-tetrahydro-3,5,5,8,8-pentamethyl-2-naphthalenyl)-1-propen-1-yl] thiophene-2-carboxylic acid.

U.S. Pat. No. 5,665,367 to Burger et al. discloses retinyl linoleate.

U.S. Pat. No. 5,587,637 to Reichert et al. discloses retinoids including (E,E)-5-[2-methyl-4-(2,6,6-trimethyl-1-cyclohexen-1-yl)-1,3-butadienyl]-2-thiophene carboxylic acid; 2-(5,6,7,8-tetrahydro-5,5,8,8-tetramethyl-2-naphthyl)-6-benzo(b)furane carboxylic acid; 2-(5,6,7,8-tetrahydro-5,5,8,8-tetramethyl-2-naphthyl)-6-indole carboxylic acid; 2-[3-(1-adamantyl)-4-methoxyphenyl]-5-benzimidazole carboxylic acid; 2-(5,6,7,8-tetrahydro-5,5,8,8-tetramethyl-2-naphthyl)-5-benzimidazole carboxylic acid; p-(5,6,7,8-tetrahydro-5,5,8,8-tetramethyl-2-1H-benz[f]indolyl)benzoic acid; and 5-(5,6,7,8-tetrahydro-5,5,8,8-tetramethyl-2-naphthoyloxymethyl)-2-thiophene carboxylic acid.

U.S. Pat. No. 5,556,844 to Reichert et al. discloses retinoids including (all-E)-9-(4-methoxy-2,3,6-trimethylphenyl)-3,7-dimethyl-2,4,6,8-nonatetraenoic acid; (E,E,E)-7-(2,3,-dihydro-1,1,3,3-tetramethyl-1H-inden-5-yl)-3,7-dimethyl-2,4,6-octatrienoic acid; (E,E,E)-7-(5,6,7,8-tetrahydro-5,5,8,8-tetramethyl-2-naphthalenyl)-3,7-dimethyl-2,4,6-octatrienoic acid; (E)-4-[(2,3,-dihydro-1,1,3,3-tetramethyl-1H-inden-5-yl)-1-propenyl] benzoic acid; (E)-4-[2-(5,6,7,8-tetrahydro-5,5,8,8-tetramethyl-2-naphthalenyl)-1-propenyl] benzoic acid; (E)-4-[2-(5,6,7,8-tetrahydro-3-methyl-5,5,8,8-tetramethyl-2-naphthalenyl)-1-propenyl] benzoic acid; 2-(5,6,7,8-tetrahydro-5,5,8,8-tetramethyl-2-naphthyl)-6-naphthalene carboxylic acid; (E)-4-[2-(5,6,7,8-tetrahydro-5,5,8,8-tetramethyl-2-naphthalenyl)-1-propenyl] benzenesulfonic acid; (E,E)-4-[2-methyl-4-(2,6,6-trimethyl-1-cyclohexen-1-yl)-1,3-butadienyl] benzoic acid; (E,E)-4-[4-methyl-6-(2,6,6-trimethyl-1-cyclohexen-1-yl)-1,3,5-hexatrienyl] benzoic acid; (E)-6-[2-(2,6,6-trimethyl-1-cyclohexen-1-yl)-ethenyl]-2-naphthalene carboxylic acid; (E)-4-[2-(5,6,7,8-tetrahydro-8,8-dimethyl-2-naphthalenyl)-1-propenyl] benzoic acid; 4-[(5,6,7,8-tetrahydro-5,5,8,8-tetramethyl-2-naphthenyl)ethynyl] benzoic acid; (E)-4-[2-(5,6,7,8-tetrahydro-3-methyl-5,5,8,8-tetramethyl-2-naphthalenyl)-1-propenyl] benzoic acid; 4-(5,6,7,8-tetrahydro-5,5,8,8-tetramethyl-2-naphthalenylcarbamoyl) benzoic acid; 4-(5,6,7,8-tetrahydro-5,5,8,8-tetramethyl-2-naphthamido) benzoic acid; (E)-4-[3-(3,5-ditert-butylphenyl)-3-oxo-1-propenyl] benzoic acid; 6-[(5,6,7,8-tetrahydro-5,5,8,8-tetramethyl-2-naphthalenyl) ethynyl] 3-pyridine carboxylic acid; 2-(5,6,7,8,-tetrahydro-5,5,8,8-tetramethyl-2-naphthyl)-6-benzo(b) thiophene carboxylic acid; 4-(5,6,7,8-tetrahydro-5,5,8,8-tetramethyl-2-anthracenyl) benzoic acid; 6-[3-(1-adamantyl)-4-methoxyphenyl]-2-naphthoic acid; 4-[3-(1-adamantyl)-4-methoxybenzamido] benzoic acid; 4-[3-(1-adamantyl)-4-methoxy benzoylthio] benzoic acid; 4-[3-(1-adamantyl)-4-methoxy benzoyloxy] benzoic acid; 2-(5,6,7,8-tetrahydro-5,5,8,8-tetramethyl-2-naphthyl)-6-carbonyl naphthalene carboxylic acid; trans-(5,5,8,8-tetramethyl-5,6,7,8-tetrahydro-2-naphthyl)-4-carbonyl-α-methyl cinnamic acid; 4-[3-(1-adamantyl)-4-methoxybenzoyloxy]-2-fluorobenzoic acid; 4-[3-(1-adamantyl)-4-methoxybenzoyloxy]-2-methylbenzoic acid; 4-[3(1-adamantyl)-4-methoxybenzoyloxy]-2-hydroxybenzoic acid; 4-[5-(1-adamantyl)-2-fluoro-4-methoxybenzoyloxy] benzoic acid; 4-[3,5-di-t-butyl-4-hydroxybenzoyloxy] benzoic acid; 4-[3-(1-adamantyl)-4-vinylbenzoyloxy] benzoic acid; 4-[3-(1-adamantyl)-4-ethylbenzoyloxy] benzoic acid; 4-[3-(1-adamantyl)-4-allyloxybenzoyloxy] benzoic acid; 4-[3-(1-adamantyl-4-methylthiobenzoyloxy] benzoic acid; 4-(5,6,7,8,-tetrahydro-5,5,8,8-tetramethyl-2-naphthylglyoxyloyloxy) benzoic acid; 4-(5,6,7,8-tetrahydro-5,5,8,8-tetramethyl-2-naphthoyloxymethyl) benzoic acid; 4-(3,5-di-t-butyl-4-hydroxybenzoyloxymethyl) benzoic acid; 4-(3-t-butyl-4-m ethoxybenzoyloxymethyl) benzoic acid; 4-(5,6,7,8-tetrahydro-5,5,8,8-tetramethyl-2-naphthoylmethyloxy) benzoic acid; 4-[1-(5,6,7,8-tetrahydro-5,5,8,8-tetramethyl-2-naphthoyloxy)ethyl] benzoic acid; 4-[[(5,6,7,8-tetrahydro-5,5,8,8-tetramethyl-2-naphthyl)ethyloxy]carbonyl] benzoic acid; 4-(5,6,7,8-tetrahydro-5,5,8,8-tetramethyl-2-naphthoylmethylamino) benzoic acid; 4-(5,6,7,8-tetrahydro-5,5,8,8-tetramethyl-2-naphthoyl formamido)benzoic acid; 4-(α-hydroxy-5,6,7,8-tetrahydro-5,5,8,8-tetramethyl-2-naphthylacetamido) benzoic acid; 4-(α-fluoro-5,6,7,8-tetrahydro-5,5,8,8-tetramethyl-2-naphthylacetamido) benzoic acid; 6-[3-(1-adamantyl)-4-(2,3-dihydroxypropyloxy) phenyl]-2-naphthoic acid; 6-[3-(1-adamantyl)-4-(3-hydroxypropyloxy)phenyl]-2-naphthoic acid; 6-[3-(1-adamantyl)-4-acetoxymethylphenyl]-2-naphthoic acid; 6-[3-(1-adamantyl)-4-methoxycarbonylphenyl]-2-naphthoic acid; 6-[3-(1-adamantyl)-4-methoxycarbonylethylphenyl]-2-naphthoic acid; 6-[3-(1-adamantyl)-4-(2-hydroxypropyl) phenyl]-2-naphthoic acid; 2-hydroxy-4-[2-hydroxy-2-(5,6,7,8-tetrahydro-5,5,8,8-tetramethyl-2-naphthyl) ethoxy] benzoic acid; methyl 2-hydroxy-4-[2-hydroxy-2-(5,6,7,8-tetrahydro-5,5,8,8-tetramethyl-2-naphthyl) ethoxy] benzoate; 2-hydroxy-4-[2-hydroxyimino-2-(5,6,7,8-tetrahydro-5,5,8,8-tetramethyl-2-naphthyl) ethoxy]benzoic acid; 2-acetyloxy-4-[2-acetyloxy-2-(5,6,7,8-tetrahydro-5,5,8,8-tetramethyl-2-naphthyl) ethoxy] benzoic acid; 2-hydroxy-4-[2-acetyloxy-2-(5,6,7,8-tetrahydro-5,5,8,8-tetramethyl-2-naphthyl) ethoxy] benzoic acid; 2-acetyloxy-4-[2-hydroxy-2-(5,6,7,8-tetrahydro-5,5,8,8-tetramethyl-2-naphthyl) ethoxy] benzoic acid; 4-(N-methyl-5,6,7,8-tetrahydro-5,5,8,8-tetramethyl-2-naphthylcarboxaminidino) benzoic acid; 4-(5,6,7,8-tetrahydro-5,5,8,8-tetramethyl-2-naphthylcarboxamidino) benzoic acid.

U.S. Pat. No. 5,235,076 to Asato et al. discloses azulenic retinoid compounds.

U.S. Pat. No. 5,158,773 to Gross discloses the retinoid ethylidene acetate 3,7-dimethyl-9-(2,6,6-trimethyl-1-cyclohexen-1-yl)-2(Z),5,7,8(E)-nonatetraenoate.

U.S. Pat. No. 5,094,783 to Muccio et al. discloses retinoids that are compounds of Formula (R-IV) and (R-V):

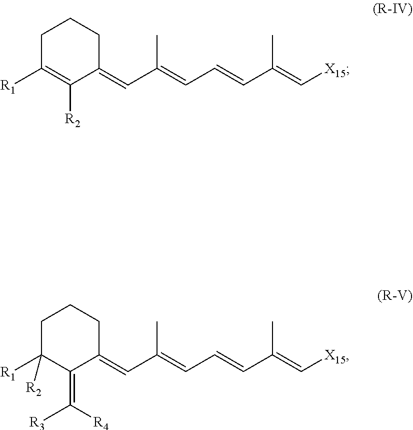

wherein:

(1) X is CO₂H or CH₂OH;

(2) in formula (R-IV), R₁ and R₂ are each hydrogen, C₁-C₄ alkyl, alkoxy, or R₁ and R₂ taken together form a 5- to 7-membered cycloalkyl or cycloalkenyl ring; and

(3) in formula (R-V), R₁ and R₂ are each hydrogen, C₁-C₄ alkyl, C₁-C₄ alkenyl or C₁-C₄ alkoxy, or R₂ and R₃ taken together form a 5- to 7-membered cycloalkyl or cycloalkenyl with R₁ and R₄ being hydrogen, C₁-C₄ alkyl, C₁-C₄ alkenyl or C₁-C₄ alkoxy.

U.S. Pat. No. 4,565,863 to Bollag et al. discloses retinoid carbohydrate derivatives, wherein the carbohydrate is a pentose, hexose, disaccharide, lower alkyl glycoside, amino sugar, amino sugar with one or more acylated amino or hydroxy groups, deoxy sugar, or sugar wherein at least two of the free hydroxy groups are acetalized.

U.S. Pat. No. 4,473,503 to Barua et al. discloses 15-fluororetinoids.

United States Patent Application Publication No. 2017/0072060 by Yu discloses prodrugs of retinoids comprising: (a) a functional unit; (b) a linker; and (c) a transportational unit; wherein the functional unit is covalently linked to the transportational unit via the linker; wherein the functional unit comprises a moiety of the retinoid; wherein the transportational unit comprises a protonatable amine group; and wherein the linker comprises a chemical bond that is capable of being cleaved after the high penetration composition penetrates across a biological barrier.

United States Patent Application Publication No. 2016/0317579 by De The et al. discloses a benzoic acid-terminated retinoid or a heterocyclic analog thereof, a napthalenecarboxylic acid terminated retinoid, and a carboxylic acid retinoid.

United States Patent Application Publication No. 2016/0287507 by Lewis II discloses conjugates of a retinoid, an organic acid, particularly an α-hydroxy acid, and an alcohol or acyl group; the retinoid, organic acid, and alcohol/acyl group are preferably linked via ester bonds.

United States Patent Application Publication No. 2015/0342920 by Mallard discloses 6-[3-(1-adamantyl)-4-hydroxyphenyl]-2-naphthoic acid, 6-[3-(1-adamantyl)-4-decyloxyphenyl]-2-napthoic acid; and 6-[3-(1-adamantyl)-4-hexyloxyphenyl]-2-naphthoic acid as retinoids.

In another alternative, the skin care agent is a prodrug of a retinoid. Suitable prodrugs of retinoids are as described above; additional prodrugs of retinoids can be constructed by employing suitable functional groups in retinoids for conjugation. As used herein, the term “prodrug” refers to compounds that are transformed in vivo to yield a disclosed compound or a pharmaceutically acceptable form of the compound. In some embodiments, a prodrug is a compound that may be converted under physiological conditions or by solvolysis to a biologically active compound as described herein. Thus, the term “prodrug” refers to a precursor of a biologically active compound that is pharmaceutically acceptable. A prodrug can be inactive when administered to a subject, but is then converted in vivo to an active compound, for example, by hydrolysis (e.g., hydrolysis in blood or a tissue). In certain cases, a prodrug has improved physical and/or delivery properties over a parent compound from which the prodrug has been derived. The prodrug often offers advantages of solubility, tissue compatibility, or delayed release in a mammalian organism (H. Bundgard, Design of Prodrugs (Elsevier, Amsterdam, 1988), pp. 7-9, 21-24). A discussion of prodrugs is provided in T. Higuchi et al., “Pro-Drugs as Novel Delivery Systems,” ACS Symposium Series, Vol. 14 and in E. B. Roche, ed., Bioreversible Carriers in Drug Design (American Pharmaceutical Association & Pergamon Press, 1987). Exemplary advantages of a prodrug can include, but are not limited to, its physical properties, such as enhanced water solubility for parenteral administration at physiological pH compared to the parent compound or enhanced drug stability for long-term storage. The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound in such a way that the modifications are cleaved, either in routine manipulation or in vivo, to yield the parent therapeutically active compound. Prodrugs include compounds wherein a hydroxy, amino, or mercapto group is covalently bonded to any group that, when the prodrug of the active compound is administered to a subject, cleaves to form a free hydroxy, free amino, or free mercapto group, respectively. Examples of prodrugs include, but are not limited to, formate or benzoate derivatives of an alcohol or acetamide, formamide or benzamide derivatives of a therapeutically active agent possessing an amine functional group available for reaction, and the like. For example, if a therapeutically active agent or a pharmaceutically acceptable form of a therapeutically active agent contains a carboxylic acid functional group, a prodrug can comprise an ester formed by the replacement of the hydrogen atom of the carboxylic acid group with a group such as C₁-C₈ alkyl, C₂-C₁₂ alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino-, or morpholino(C₂-C₃)alkyl. Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆))alkanoyloxy)ethyl, 1-methyl-1-((C₁-C₆)alkanoyloxy)ethyl (C₁-C₆)alkoxycarbonyloxymethyl, N(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate). If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, C(OH)C(O)OY¹ wherein Y¹ is H, (C₁-C₆)alkyl or benzyl, C(OY²)Y³ wherein Y² is (C₁-C₄) alkyl and Y³ is (C₁-C₆)alkyl, carboxy(C₁-C₆)alkyl, amino(C₁-C₄)alkyl or mono-N or di-N,N(C₁-C₆)alkylaminoalkyl, C(Y⁴)Y⁵ wherein Y⁴ is H or methyl and Y⁵ is mono-N or di-N,N(C₁-C₆)alkylamino, morpholino, piperidin-1-yl or pyrrolidin-1-yl. The use of prodrug systems is described in T. Järvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796.

Hydroxyacids used in skin care products are typically α-hydroxyacids, and include, but are not limited to, glycolic acid, lactic acid, malic acid, citric acid, and tartaric acid. Other hydroxyacids include tartronic acid, glucuronic acid, pyruvic acid, 2-hydroxyisobutyric acid, 3-hydroxybutyric acid, galacturonic acid, mandelic acid, mucic acid, α-phenyllactic acid, α-phenylpyruvic acid, saccharic acid, α-hydroxybutyric acid, α-hydroxyisobutyric acid, α-hydroxyisocaproic acid, α-hydroxyisovaleric acid, atrolactic acid, galactaric acid, pantoic acid, glyceric acid, isocitric acid, dihydroxymaleic acid, dihydroxytartaric acid, dihydroxyfumaric acid, and benzylformic acid. The hydroxyacid can also be a β-hydroxyacid such as salicylic acid or a derivative thereof such as octanoyl salicylate.

U.S. Pat. No. 8,158,811 to Charveron et al. discloses additional hydroxyacids such as 10-hydroxy-dec-2-fluro-2-enoic acid, 12-hydroxy-dodeca-2-fluoro-2-enoic acid, 14-hydroxy-tetradec-2-enoic acid, 14-hydroxy-tetradec-2-fluoro-2-enoic acid, and 17-hydroxy-heptadec-2-fluoro-2-enoic acid.

U.S. Pat. No. 6,399,082 to Ganemo discloses glucuronic acid and pyruvic acid as hydroxyacids.

U.S. Pat. No. 5,961,999 to Bimczok et al. discloses saccharic acid as a hydroxyacid.

U.S. Pat. No. 5,686,489 to Yu et al. discloses hydroxyacid esters including partial or full esters.

United States Patent Application Publication No. 2006/0057075 by Arkin et al. discloses additional hydroxyacids including agaricic acid, aleuritic acid, allaric acid, altraric acid, arabiraric acid, ascorbic acid, benzilic acid, citramalic acid, erythraric acid, glucaric acid, glyceric acid, gularic acid, gulonic acid, hydroxypyruvic acid, idaric acid, lyxaric acid, mannaric acid, methyllactic acid, quinic acid, ribaric acid, ribonic acid, talaric acid, threaric acid, tropic acid, uronic acid, and xylaric acid.

Other hydroxyacids suitable for use in skin care preparations, including both α-hydroxyacids and β-hydroxyacids, are known in the art.

In another alternative, the skin care agent can be an ester of a hydroxyacid, such as an ester of an α-hydroxyacid or a β-hydroxyacid.

Cosmeceuticals include, but are not limited to the following components and compositions.

U.S. Pat. No. 9,381,145 to Sambanthamurthi et al. discloses botanical extracts from oil palm vegetation liquor for cosmeceutical applications, including phenolic compounds, fruit acids, and sugars.

U.S. Pat. No. 8,673,859 to Brem et al. discloses GM-CSF or nucleic acids expressing GM-CSF for cosmeceutical applications.

U.S. Pat. No. 8,568,751 to Goldsberry et al. discloses a cosmeceutical composition including a suspension of a powder of an aliphatic polyester copolymer, a cross-linked silicone elastomer, and at least one hydrolysate or acylated short-chain peptide.

U.S. Pat. No. 8,524,292 to Kopas et al. discloses a cosmeceutical composition including a mixture of refined, bleached, deodorized palm oils and red palm olein.

U.S. Pat. No. 8,193,196 to Majeed et al. discloses a cosmeceutical composition including a dipeptide incorporating a selenoamino acid.

U.S. Pat. No. 7,550,603 to Hong et al. discloses a cosmeceutical composition including 3,6-dihydro-2H-pyrans.

U.S. Pat. No. 7,300,649 to Tajono et al. discloses a cosmeceutical composition including calcium chloride, magnesium chloride, and potassium bromide for restoration of skin barrier function.

U.S. Pat. No. 6,149,896 to Riklis et al. discloses a cosmeceutical composition including nordihydroguiaretic acid, niacinimide, and, optionally, an antioxidant such as propyl gallate.

United States Patent Application Publication No. 2014/0206842 by Majeed et al. discloses a cosmeceutical composition including a peptide modified with a triterpenoid.

United States Patent Application Publication No. 2010/0255080 by Sanmiguel et al. discloses a cosmeceutical composition including 5-aminolevulinic acid.

United States Patent Application Publication No. 2010/0240767 by Majeed et al. discloses a cosmeceutical composition including 3,5-dimethoxy-4′-hydroxystilbene.

United States Patent Application Publication No. 2009/0221625 by Hirsch et al. discloses cosmeceutical compositions including alkanediols including propyleneglycol (1,2-propanediol), butyleneglycol, 2-ethyl-1,3-hexanediol, or hexyleneglycol (2-methyl-2,4-pentanediol); ether diols including dipropyleneglycol or diethyleneglycol; or diether alcohols including diethyleneglycol monoethylether.

United States Patent Application Publication No. 2008/0317733 by Azimi discloses cosmeceutical compositions including hyaluronic acid, kokic acid, and glycolic acid.

United States Patent Application Publication No. 2006/0110481 by Majeed et al. discloses cosmeceutical compositions including artemetin.

United States Patent Application Publication No. 2006/0057075 by Arkin et al. discloses cosmeceutical compositions including at least one of: (1) hydroquinone or derivatives thereof as age spot/keratosis remover; (2) anti-acne agents, including N-acetylcysteine, adapalene, azelaic acid, benzoyl peroxide, cholate, clindamycin, deoxycholate, erythromycin, flavonoids, glycolic acid, meclocycline, mupirocin, octopirox, phenoxyethanol, phenoxypropanol, pyruvic acid, resorcinol, retinoic acid, salicylic acid, scymnol sulfate, sulfacetamide-sulfur, sulfur, tazarotene, tetracycline, or tretinoin triclosan; (3) melatonin as an anti-aging agent; (4) antipsoriatic agents including 6-aminonicotinam ide, 6-aminonicotinic acid, 2-aminopyrazinamide, anthralin, calcipotriene, 6-carbamoylnicotinamide, 6-chloronicotinamide, 2-carbamoylpyrazinam ide, corticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, or trichothecene mycotoxins; (5) antirosacea agents including azelaic acid or metronidazole sulfacetamide; (6) hydroxyacids including agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllactic acid, mucic acid, phenyllactic acid, pyruvic acid, quinic acid, ribaric acid, ribonic acid, saccharic acid, talaric acid, tartaric acid, tartronic acid, threaric acid, tropic acid, uronic acids, or xylaric acid; (7) non-steroidal anti-inflammatory agents including: oxicams, including ampiroxicam, cinnoxicam, droxicam, piroxicam, isoxicam, lornoxicam, meloxicam, tenoxicam, sudoxicam; nabumetone; Cox-2 inhibitors including rofecoxib, celecoxib, cimicoxib, deracoxib, etoricoxib, imrecoxib, lumaricoxib, parecoxib, tilmacoxib, valdecoxib; nimesulide; 4-hydroxy-2-methyl-N-phenyl-2H-1,2-benzothiazixine-3-carboxamide-1,1-dioxide (CP-14,304); cinmetacin; clonixin; epirizole; ethenzamide; fenclozic acid; fenclozine; filenadol; orpanoxin; oxaceprol; oxaprozin; parsalmide; salicin; satigrel; talmetacin; triflusal; tropesin; ursolic acid; zidometacin; salicylates, including sodium salicylate and choline magnesium trisalicylate, acetylsalicylic acid (aspirin), salsalate, diflunisal, sulfasazaline, olsalazine, salicylamide, salicylsalicylic acid, salicylic acid; acetaminophen; indomethacin; benzydamine; benzpiperylon; bucloxic acid; bumadizone; darbufelone; florifenine; flosulide; flubichin methanesulfonate; flufenisal; flunixin; licofelone; pamicogrel; parcetasal; sulindac; talniflumate; tazofelone; tebufelone; tenidap; tepoxalin; tiaramide; tinoridine; benorylate; trilisate; fendosal; acetic acid derivatives, including aceclofenac, alclofenac, amfenac, bendazac, bromfenac, clidanac, clopirac, diclofenac, eltenac, etodolac, felbinac, fenclofenac, tolmetin, isoxepac, furofenac, ibufenac, isofezolac, isoxepac, lonazolac, mofezolac, nepafanac, nitrofenac, pemedolac, pirazolac, tiopinac, acemetacin, fentiazac, zomepirac, clindanac, oxindanac, oxepinac, felbinac, ibufenac, ketorolac; fenamates, including erfenamic acid, etofenamate, mefenamic acid, meclofenamic acid, flufenamic acid, niflumic acid, tolfenamic acid; propionic acid derivatives, including araprofen, alminoprofen, bermoprofen, butibufen, ibuprofen, naproxen, benoxaprofen, esflurbiprofen, flurbiprofen, ketoprofen, fenoprofen, fenbufen, flobufen, flunoxaprofen, fluprofen, indoprofen, lobuprofen, loxaprofen, mabuprofen, miroprofen, naproxen, nitroflurbiprofen, nitronaproxen, pelubiprofen, pirprofen, carprofen, pranoprofen, miroprofen, tiaprofenic acid, tioxaprofen, suprofen, alminoprofen, tiaprofen, ximoprofen, zaltoprofen; pyrazoles; phenylbutazone; oxyphenbutazone; apazone; clofezone; feprazone; fluproquazone; mofebutazone; morazone; azapropazone; and trimethazone; (8) histamine receptor H₁ antagonists such as, but not limited to, doxepin hydrochloride, carbinoxamine maleate, clemastine fumarate, diphenhydramine hydrochloride, dimenhydrinate, pyrilamine citrate, tripelennamine hydrochloride, tripelennamine citrate, chlorpheniramine mdialeate, brompheniramine maleate, hydroxyzine hydrochloride, hydroxyzine pamoate, cyclizine hydrochloride, cyclizine lactate, meclizine hydrochloride, promethazine hydrochloride, cyproheptadine hydrochloride, phenindamine tartrate, acrivastine, cetirizine hydrochloride, azelastine hydrochloride, levocabastine hydrochloride, loratidine, desloratidine, ebastine, mizolastine, and fexofenadine, and the salts, solvates, analogues, congeners, bioisosteres, hydrolysis products, metabolites, precursors, and prodrugs thereof, as well as additional ethanolamines, alkylamines, ethylenediamines, piperazines, phenothiazines, and tricyclic piperidines that are histamine receptor H₁ antagonists; (9) histamine receptor H₂ antagonists such as, but not limited to cimetidine, ranitidine, and the salts, solvates, analogues, congeners, bioisosteres, hydrolysis products, metabolites, precursors, and prodrugs thereof; (10) histamine receptor H₃ antagonists and the salts, solvates, analogues, congeners, bioisosteres, hydrolysis products, metabolites, precursors, and prodrugs thereof; and (11) histamine receptor H₄ antagonists, including but not limited to 5-chloro-2-[(4-methylpiperazin-1-yl)carbonyl]-1H-indole (JNJ7777120) and the salts, solvates, analogues, congeners, bioisosteres, hydrolysis products, metabolites, precursors, and prodrugs thereof.

Additionally, anti-inflammatory agents suitable for incorporation into a targeting composition according to the present invention include kinin receptor antagonists, including, but not limited to, B₁ or B₂ receptor antagonists and the salts, solvates, analogues, congeners, bioisosteres, hydrolysis products, metabolites, precursors, and prodrugs thereof. A number of kinin receptor antagonists, including peptides containing one or more amino acids of the D-configuration and small molecules, are disclosed in United States Patent Application Publication No. 2008/0221039 by Gibson et al.

Additionally, anti-inflammatory agents suitable for incorporation into a targeting composition according to the present invention include leukotriene receptor antagonists such as zafirlukast and montelukast, and the salts, solvates, analogues, congeners, bioisosteres, hydrolysis products, metabolites, precursors, and prodrugs thereof.

United States Patent Application Publication No. 2005/0191267 by Luanratana discloses cosmeceutical compositions including herbal plant extracts.

United States Patent Application Publication No. 2005/0079210 by Gupta discloses cosmeceutical compositions in which potential cosmeceutical ingredients can include vitamin E, vitamin E acetate, tocotrienol, progesterone, capsaicin, capsicum oleoresin, menthol, methyl salicylate, benzophenone-3, octyl methoxycinnamate, benzocaine, and lidocaine.

The Wnt signaling pathway is involved in inflammation and its modulation can be used to treat skin disorders associated with inflammation, including, but not limited to, rashes accompanied by skin itching and redness, dermatitis, eczema, rosacea, seborrheic dermatitis, and psoriasis. The Wnt signaling pathway is activated by binding a Wnt-protein ligand to a Frizzled family receptor, which passes the biological signal to the Dishevelled protein to the cell, regulating gene transcription. Wnt comprises a diverse family of secreted lipid-modified signaling glycoproteins that are 350-400 amino acids in length. The type of lipid modification that occurs on these proteins is palmitoylation of cysteines in a conserved pattern of 23-24 cysteine residues. Palmitoylation is necessary because it initiates targeting of the Wnt protein to the plasma membrane for secretion and it allows the Wnt protein to bind its receptor due to the covalent attachment of fatty acids. Wnt proteins also undergo glycosylation, which attaches a carbohydrate in order to ensure proper secretion. In Wnt signaling, these proteins act as ligands to activate the different Wnt pathways via paracrine and autocrine routes.

Accordingly, agents that modulate the Wnt signaling pathway can be used as cosmeceuticals or skin care products, or as components of cosmeceutical or skin care products.

Wnt modulators include, but are not limited to: XAV-939 (3,5,7,8-tetrahydro-2-[4-(trifluoromethyl)phenyl]-4H-thiopyrano[4,3-d]pyrimidin-4-one); ICG-001 (an enantiomer of PRI-724 ((6S,9a5)-N-benzyl-6-[(4-hydroxyphenyl)methyl]-8-(naphthalen-1-ylmethyl)-4,7-dioxo-3,6,9,9a-tetrahydro-2H-pyrazino[1,2-a]pyrimidine-1-carboxamide)); IWR-1-endo (4-[(3ar,4s,7r,7as)-1,3,3a,4,7,7a-hexahydro-1,3-dioxo-4,7-methano-2h-isoindol-2-yl]-N-8-quinolinyl-benzamide); Wnt-059 (2-[4-(2-methylpyridin-4-yl)phenyl]-N-(4-pyridin-3-ylphenyl)acetamide); LKG-974 (2-[5-methyl-6-(2-methylpyridin-4-yl)pyridin-3-yl]-N-(5-pyrazin-2-ylpyridin-2-yl)acetamide); iCRT3 (2-[[2-(4-ethylphenyl)-5-methyl-1,3-oxazol-4-yl]methylsulfanyl]-N-(2-phenylethyl)acetamide); GNF-6231 (N-(5-(4-acetylpiperazin-1-yl)pyridin-2-yl)-2-(2′,3-dimethyl-[2,4′-bipyridin]-5-yl)acetamide); methyl vanillate; LF3 (4-[(E)-3-phenylprop-2-enyl]-N-(4-sulfamoylphenyl)piperazine-1-carbothioamide); CP21R7 (3-(3-aminophenyl)-4-(1-methyl-1H-indol-3-yl)-1H-pyrrole-2,5-dione); NCB-0846; PNU-74654 (N-[(5-methylfuran-2-yl)methylideneamino]-2-phenoxybenzamide); KY02111 (N-(6-chloro-1,3-benzothiazol-2-yl)-3-(3,4-dimethoxyphenyl)propanamide); IWP-2 (N-(6-methyl-1,3-benzothiazol-2-yl)-2-[(4-oxo-3-phenyl-6,7-dihydrothieno[3,2-d]pyrimidin-2-yl)sulfanyl]acetamide); salinomycin; FH535 (2,5-dichloro-N-(2-methyl-4-nitrophenyl)benzenesulfonamide); WIKI4 (2-[3-[[4-(4-methoxyphenyl)-5-pyridin-4-yl-1,2,4-triazol-3-yl]sulfanyl]propyl]benzo[de]isoquinoline-1,3-dione); PRI-724; and KYA1797K ((Z)-3-(5-((5-(4-nitrophenyl)furan-2-yl)methylene)-4-oxo-2-thioxothiazolidin-3-yl)propanoic acid).

U.S. Pat. No. 9,771,351 to Ishida et al. discloses Wnt inhibitors, including compounds of Formula (W-I):

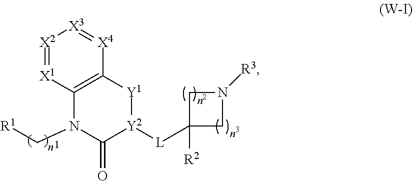

wherein:

(1) n¹ is 0 or 1;

(2) n² and n³ are the same or different, and each is 1 or 2;

(3) when n¹ is 0, R¹ is an optionally substituted aryl, an optionally substituted aromatic heterocyclic group, or an optionally substituted aliphatic heterocyclic group, and when n¹ is 1, R¹ is an aryl substituted with cyano or carbamoyl, an optionally substituted aromatic heterocyclic group, or an optionally substituted aliphatic heterocyclic group;

(4) R² is a hydrogen atom or hydroxy;

(5) R³ is an optionally substituted aromatic heterocyclic group or an optionally substituted aliphatic heterocyclic group;

(6) X¹, X², X³, and X⁴ may be the same or different, and each is N or CR⁴; each R⁴ independently is hydrogen, lower alkyl, cyano, halogen, hydroxy, lower alkoxy, lower alkanoyl, or lower alkylsulfonyl;

(7) Y¹ is CH₂ or C(═O);

(8) Y² is CH or N; and

(9) L is CH₂ or NH.

U.S. Pat. Nos. 9,763,927, 9,381,192, 9,199,991, 8,987,298, 8,822,478, 8,703,794, 8,673,936, 8,664,241, and 8,604,052 to Hood et al. disclose indazole derivatives as Wnt inhibitors.

U.S. Pat. No. 9,649,300 to Rebbaa et al. discloses Wnt inhibitors, including compounds of Formula (W-II):

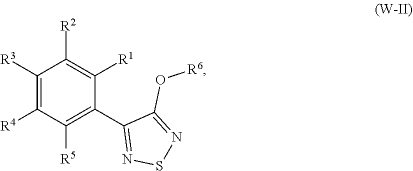

wherein:

(1) each of R¹, R², R³, R⁴, and R⁵ is individually selected from H, halogen, optionally substituted alkyl, optionally substituted alkoxy, nitro, sulfonamide, hydroxy, or amino; and

(2) R⁶ is selected from H, optionally substituted alkyl, or optionally substituted aryl.

U.S. Pat. No. 9,579,361 to Satyal et al. discloses Wnt-binding polypeptides as Wnt inhibitors.

U.S. Pat. No. 9,556,144 An et al. discloses Wnt inhibitors, including compounds of Formula (W-III):

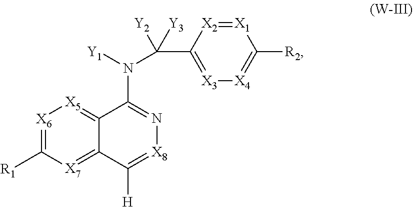

wherein:

(1) X₁, X₂, X₃, X₄, X₅, X₆, X₇, and X₅ are independently CR₄ or N;

(2) Y₁ is hydrogen or —C(R₄)₃, wherein each R₄ is the same or different;

(3) Y₂ and Y₃ are independently hydrogen, halogen or —C(R₃)₃, wherein each R₃ is the same or different;

(4) R₁ and R₂ are independently selected from hydrogen, halogen, C₁-C₆ alkyl, quinolinyl, a moiety of Formula (W-III(a)),

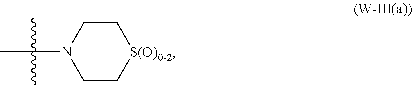

C₆-C₃₀ aryl, 3 to 6 membered heterocycloalkyl containing 1-2 heteroatoms selected from N, O and S, and 5 or 6 membered heteroaryl containing 1-4 heteroatoms selected from N, O and S, wherein each of quinolinyl, the moiety of Formula (W-III(a)), C₆-C₃₀ aryl, 3 to 6 membered heterocycloalkyl, and 5 or 6 membered heteroaryl can be optionally substituted with one or two R₄, which can be the same or different;

(5) each R₃ is independently selected from hydrogen, halogen, cyano, C₁-C₆ alkyl, and C₁-C₆ alkoxy, wherein each of the C₁-C₆ alkyl and C₁-C₆ alkoxy can be optionally substituted with halo, amino, hydroxyl, C₁-C₆ alkoxy or cyano;

(6) R₄ is independently selected from hydrogen, halogen, cyano, C₁-C₆ alkoxy, —S(O)₂R₅, —C(O)OR₅, —C(O)R₅, —C(O)NR₆R₇, C₁-C₆ alkyl, C₂-C₆ alkenyl and C₂-C₆ alkynyl, wherein each of C₁-C₆ alkoxy, —S(O)₂R₅, —C(O)OR₅, —C(O)R₅, —C(O)NR₆R₇, C₁-C₆ alkyl, C₂-C₆ alkenyl and C₂-C₆ alkynyl can be optionally substituted with halo, amino, hydroxyl, C₁-C₆ alkoxy or cyano; and

(7) R₅, R₆ and R₇ are independently selected from hydrogen, C₁-C₆ alkyl, C₂-C₆ alkenyl, and C₂-C₆ alkynyl, in which each of the C₁-C₆ alkyl, C₂-C₆ alkenyl, and C₂-C₆ alkynyl can be optionally substituted with halo, amino, hydroxyl, C₁-C₆ alkoxy or cyano.

U.S. Pat. No. 9,427,457 to Komuro et al. discloses the use of an insulin-like growth factor-binding protein as a Wnt inhibitor.

U.S. Pat. No. 9,403,812 to Holsworth et al. discloses triazole derivatives as Wnt inhibitors.

U.S. Pat. No. 9,359,444 to Dupont et al. discloses antibodies binding a Frizzled protein as Wnt inhibitors.

U.S. Pat. No. 9,303,087 to Ma et al. discloses monoclonal antibodies against LRP96 as Wnt inhibitors.

U.S. Pat. No. 9,238,646 to Cheng et al. discloses (N-(hetero)aryl, 2-(hetero)aryl-substituted acetamides as Wnt inhibitors.

U.S. Pat. No. 9,181,235 to Cheng et al. discloses substituted pyridines as Wnt inhibitors.

U.S. Pat. No. 9,056,891 to Tarasova et al. discloses synthetic peptides as Wnt inhibitors.

U.S. Pat. No. 9,045,416 to Lum et al. discloses Wnt inhibitors, including the compound of Formula (W-IV):

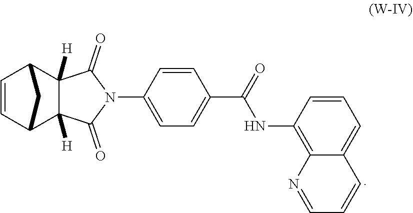

U.S. Pat. No. 7,723,477 to Gurney et al. discloses soluble FZD receptor as a Wnt inhibitor.

U.S. Pat. No. 7,652,043 to Beachy et al. discloses Wnt inhibitors, including the compound of Formula (W-V):

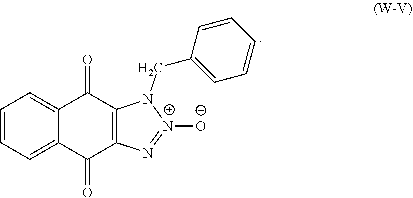 [SMILES: O=C1c2ccccc2C(=O)c2c1n[n+]([O-])n2Cc1ccccc1]

United States Patent Application Publication No. 2017/0298062 by Bhamra et al. discloses N-pyridinylacetamide derivatives as Wnt inhibitors.

United States Patent Application Publication No. 2017/0210741 by Augelli-Szafran et al. discloses benzimidazole compounds as Wnt inhibitors.

United States Patent Application Publication No. 2017/0114070 by Thede et al. discloses substituted trifluoromethoxybenzamides as Wnt inhibitors.

United States Patent Application Publication No. 2016/0355496 by Boutros et al. discloses chromene derivatives as Wnt inhibitors.

United States Patent Application Publication No. 2016/0318926 by Alam et al. discloses maleimide derivatives as Wnt inhibitors.

United States Patent Application Publication No. 2016/0311829 by Alam et al. discloses dihydropyrazolo[1,5-a]pyrimidines as Wnt inhibitors.

United States Patent Application Publication No. 2016/0090386 by Ho et al. discloses purine diones as Wnt inhibitors.

United States Patent Application Publication No. 2015/0051177 by Orton discloses substituted benzamides as Wnt inhibitors, including N-(4-fluorophenyl)-4-(indolin-1-ylsulfonyl)benzam ide; N-(4-(N-p-tolylsulfamoyl)phenyl)-4,5-dihydro-1H-benzo[g]indazole-3-carboxamide; N-(4-(N-(2,5-dimethylphenyl)sulf-amoyl)phenyl)-4,5-dihydro-1H-benzo[g]indazole-3-carboxamide; N-(5,6-dimethoxybenzo[d]thiazol-2-yl)-4-(N-methyl-N-phenyl-sulfamoyl)benzamide; N-(5,6-dimethoxybenzo[d]thiazol-2-yl)-4-(indolin-1-ylsulfonyl)benzamide; 4-(3,4-dihydroquinolin-1(2H)-ylsulfonyl)-N-(4-(pyridin-2-yl)thiazol-2-yl)benzamide; 4-(indolin-1-ylsulfonyl)-N-(5-methyl-4-phenylthiazol-2-yl)benzamide; N-(2-carbamoylphenyl)-3-(N-phenylsulfamoyl)benzamide; 4-(3,4-dihydroquinolin-1(2H)-ylsulfonyl)-N-(4-p-tolylthiazol-2-yl)benzamide; 4-(3,4-dihydroquinolin-1(2H)-ylsulfonyl)-N-(4-phenylthiazol-2-yl)benzamide; dihydroquinolin-1(2H)-ylsulfonyl)-N-(6-methylbenzo[d]thiazol-2-yl)benzam ide; and N-(4-(4-acetamidophenyl)thiazol-2-yl)-4-(3,4-dihydroquinolin-1(2H)-ylsulfonyl)benzam ide.

United States Patent Application Publication No. 2014/0350015 by McDonald et al. discloses pyridine and pyrimidine derivatives as Wnt inhibitors.

United States Patent Application Publication No. 2014/0194441 by Kumar et al. discloses 3-(benzoimidazol-2-yl)-indazole derivatives as Wnt inhibitors, including 4-(3,4-dihydroquinolin-1(2H)-ylsulfonyl)-N-(4-(pyridin-2-yl)thiazol-2-yl)benzamide, 4-(3,4-dihydroquinolin-1(2H)-ylsulfonyl)-N-(4-p-tolylthiazol-2-yl)benzamide), 4-(3,4-dihydroquinolin-1(2H)-ylsulfonyl)-N-(4-phenylthiazol-2-yl)benzamide, 4-(3,4-dihydroquinolin-1(2H)-ylsulfonyl)-N-(6-methylbenzo[d]thiazol-2-yl)benzamide, N-(4-(4-acetamidophenyl)thiazol-2-yl)-4-(3,4-dihydroquinolin-1(2H)-ylsulfonyl)benzamide, and 3-(3,4-dihydroquinolin-1(2H)-ylsulfonyl)-N-(4-(pyridin-2-yl)thiazol-2-yl)benzamide.

Other Wnt inhibitors are known in the art.

Other suitable skin treatment products suitable for incorporation into compositions according to the present invention are disclosed in U.S. Pat. No. 8,163,298 to Griffiths-Brophy. These agents can include vitamins, peptides, aminosugars, sunscreens, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols, plant hormones, tyrosinase inhibitors, anti-inflammatory agents, N-acyl amino acid compounds, antimicrobials, and antifungals, suitable alternatives for which are disclosed in United States Patent Application Publication No. 2006/0275237 by Bissett et al. and United States Patent Application Publication No. 2004/0175347 by Bissett.

As used herein, the terms “stable,” “stability,” or equivalent terminology mean compositions which are substantially unaltered in chemical state, physical state and/or color. “Stable” further means that the compositions and the skin care actives exhibit stability under reasonable shelf storage conditions and under conditions reasonably expected to be incurred during transport and storage. Transport and storage conditions may include prolonged exposure to temperatures of from about −50° C. to about 60° C. Stability may be determined either by empirical observation or by appropriate methods of chemical analysis that would be known to one of skill in the art.

“Keratinous tissue,” as used herein, means keratin-containing layers disposed as the outermost protective covering of mammals and includes, but is not limited to, skin, hair and nails. “Topical application,” as used herein, means to apply or spread a composition onto the surface of the keratinous tissue. This may be done directly or indirectly, such as by applying the composition to a patch and applying the patch to the keratinous tissue.

As used herein, the term “skin care agent” means an agent or a composition including the agent suitable for application to or delivery to mammalian keratinous tissue and that provides a benefit or improvement to the keratinous tissue. As stated above, the keratinous tissue can, in some alternatives, include keratinous tissue other than skin.

As used herein, the term “skin care” or similar terminology means regulating and/or improving skin condition. Herein, “regulating skin condition” means improving skin appearance and/or feel, for example, by providing a smoother appearance and/or feel. Herein, “improving skin condition” or equivalent terminology means effecting a visually and/or tactilely perceptible positive change in skin appearance and feel. Conditions that may be regulated and/or improved include, but are not limited to, one or more of the following: reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, and graying hair. As used herein, “signs of skin aging,” include, but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Dermatologically acceptable” or similar terminology, as used herein, means that the compositions or components thereof so described are suitable for use in contact with mammalian keratinous tissue without undue toxicity, incompatibility, instability, allergic response, or other deleterious effects, whether or not such effects are visible to the naked eye.

As used herein, the term “delivery enhancement device” means any device that increases the amount of any active ingredient applied to and/or into the skin relative to the amount of active ingredient that is delivered without using the device or, alternatively, prevents application of any active ingredient to regions of the skin to which the delivery of the active ingredient is not desired or intended. Delivery enhancement devices can include, but are not limited to, spray applicators.

As used herein, the term “kit” or similar terminology means a packaging unit comprising at least one composition described herein. The kit may comprise an outer packaging unit, which in turn may comprise one or more inner packaging units. The inner and outer packaging units may be of any type suitable for containing, presenting and/or reasonably protecting from damage the contents of the kit. The kit may comprise one or more compositions as described herein, a delivery enhancement device, instructions for use of the device, instructions for complying with suitable application regimens, or combinations thereof.

As used herein, the term “vitamins” includes vitamins, provitamins, and their salts, isomers and derivatives. The vitamins may include those vitamins not known to exhibit significant antioxidant properties, for example, vitamin D compounds; vitamin K compounds; and mixtures thereof. The compositions of the present invention optionally may include those which exhibit antioxidant properties, non-limiting examples of suitable vitamins include: vitamin B compounds (including niacinamide, nicotinic acid, C₁-C₁₈ nicotinic acid esters, and nicotinyl alcohol; vitamin B6 compounds, such as pyroxidine; and vitamin B5 compounds, such as panthenol, or “pro-B5”); vitamin A compounds, and all natural and/or synthetic analogs of Vitamin A, including retinoids, carotenoids, and other compounds which possess the biological activity of Vitamin A; vitamin E compounds, or tocopherol, including tocopherol sorbate, tocopherol acetate, other esters of tocopherol; vitamin C compounds, including ascorbyl esters of fatty acids, and ascorbic acid derivatives, for example, ascorbyl glucoside, magnesium ascorbyl phosphate, sodium ascorbyl phosphate, and ascorbyl sorbate.

As used herein, the term “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palm itoyl-lysine-threonine-threonine-lysine-serine (pal-KTTKS), palmitoyl-glycine-glutamine-proline-arginine (pal-GQPR), and Cu-histidine-glycine-glycine (Cu-HGG). In some alternatives, preferred peptides can contain at least one basic amino acid (lysine, arginine, or histidine). Other preferred peptides include: carnosine (β-alanine-histidine), a dipeptide with the non-standard amino acid β-alanine; the tripeptide histidine-glycine-glycine; the tripeptide glycine-glycine-histidine; the tripeptide glycine-histidine-glycine; and the pentapeptide lysine-threonine-threonine-lysine-serine (KTTKS) (SEQ ID NO: 95). Still other suitable peptides include: arginine-lysine-arginine, acetyl-arginine-lysine-arginine-NH₂; and arginine-serine-arginine-lysine. Still other suitable peptides include palmitoylated peptides such as palmitoyl-lysine-threonine-threonine-lysine-serine.

The composition may also include aminosugars including glucosamine, mannosamine, galactosamine, N-acetylglucosamine, N-acetyl-glycerosamine, N-acetyl-erythrosamine, N-acetyl-threosamine, N-acetyl-ribosamine, N-acetyl-arabinosamine, N-acetyl-xylosamine, N-acetyl-lyxosamine, N-acetyl-allosamine, N-acetyl-altrosamine, N-acetyl-mannosamine, N-acetyl-gulosamine, N-acetyl-idosamine, N-acetyl-galactosamine, N-acetyl-talosamine, N-acetyl-glucoheptosamine, N-acetyl-galactoheptosamine, N-acetyl-mannoheptosamine, N-acetyllactosamine, N-acetylmuramic acid, N-acetylneuramine, N-acetylneuramin lactose, N-acetyl-glyceraminic acid, N-acetyl-erythrosaminic acid, N-acetyl-threosaminic acid, N-acetyl-ribosaminic acid, N-acetyl-arabinosaminic acid, N-acetyl-xylosaminic acid, N-acetyl-lyxosaminic acid, N-acetyl-allosaminic acid, N-acetyl-altrosaminic acid, N-acetyl-glucosaminic acid, N-acetyl-mannosaminic acid, N-acetyl-gulosaminic acid, N-acetyl-idosaminic acid, N-acetyl-galactosaminic acid, N-acetyl-talosaminic acid, N-acetyl-heptoglucosaminic acid, N-acetyl-heptogalactosaminic acid, N-acetyl-heptomannosaminic acid, N-acetyl-N-acetylneuraminic acid, and isomeric or nonisomeric, free acid, salt, lactone, amide, or ester forms thereof. Suitable aminosugars are disclosed in U.S. Pat. No. 6,159,485 to Yu et al.

The composition may also include one or more sunscreen actives (or sunscreen agents) and/or ultraviolet light absorbers. As used herein, “sunscreen active” includes both sunscreen agents and physical sunblocks. Sunscreen actives and ultraviolet light absorbers may be organic or inorganic. Sunscreens suitable for inclusion in a skin care composition include 2-ethylhexyl-p-methoxycinnamate, 4,4′-t-butyl methoxydibenzoyl-methane, 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyWaminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, menthyl anthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonylbenzoxazoic acid, octocrylene, zinc oxide, benzylidene camphor and derivatives thereof, titanium dioxide, and mixtures thereof. Other suitable sunscreens include, but are not limited to: p-aminobenzoic acid, its salts, and derivatives, including ethyl, isobutyl, and glyceryl esters; anthranilates, including methyl anthranilate, phenyl anthranilate, benzyl anthranilate, linalyl anthranilate, terpinyl anthranilate, and cyclohexenyl anthranilate; salicylates, including amyl, phenyl, octyl, benzyl, menthyl, glyceryl, and dipropyleneglycol esters; cinnamic acid derivatives, including menthyl and benzyl esters, α-phenyl cinnamonitrile, butyl cinnamoyl pyruvate; dihydroxycinnamic acid derivatives, including umbelliferone, methylumbelliferone, methylacetoumbelliferone; trihydroxycinnamic acid derivatives, including esculetin, methylesculetin, daphnetin, and the glucosides esculin and daphnin; hydrocarbons including diphenylbutadiene and stilbene; dibenzalacetone; benzalacetophenone; naphtholsulfonates; dihydroxynaphthoic acid and its salts; o- and p-hydroxybiphenyldisulfonates; coumarin derivatives, including 7-hydroxy, 7-methyl, and 3-phenyl derivatives; diazoles, including 2-acetyl-3-bromoindazole, phenyl benzoxazole, methyl naphthoxazole, and various aryl benzothiazoles; quinine salts, including bisulfate, sulfate, chloride, oleate, and tannate; quinoline derivatives, including 8-hydroxyquinoline salts and 2-phenylquinoline; hydroxy- or methoxy-substituted benzophenones; uric and violuric acids; tannic acid and its derivatives (e.g., hexaethylether); (butyl carbitol) (6-propyl piperonyl) ether; hydroquinone; benzophenones, including oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one); terephthalylidene dicamphor sulfonic acid; and 4-isopropyl-dibenzoylmethane. Other sunscreen actives are described in U.S. Pat. No. 4,937,370 to Sabatelli and U.S. Pat. No. 4,999,186 to Sabatelli et al. The sunscreen actives described in these patents have, in a single molecule, two distinct chromophore moieties which exhibit different ultra-violet radiation absorption spectra. One of the chromophore moieties absorbs predominantly in the UVB radiation range and the other absorbs strongly in the UVA radiation range. These sunscreen actives include 4-N,N-(2-ethylhexyl)methyl aminobenzoic acid ester of 2,4-dihydroxybenzophenone; N,N-di-(2-ethylhexyl)-4-aminobenzoic acid ester with 4-hydroxydibenzoylmethane; 4-N,N-(2-ethylhexyl)methyl-aminobenzoic acid ester with 4-hydroxydibenzoylmethane; 4-N,N-(2-ethylhexyl)methyl-aminobenzoic acid ester of 2-hydroxy-4-(2-hydroxyethoxy)benzophenone; 4-N,N-(2-ethylhexyl)-methylaminobenzoic acid ester of 4-(2-hydroxyethoxy)dibenzoylmethane; N,N-di-(2-ethylhexyl)-4-aminobenzoic acid ester of 2-hydroxy-4-(2-hydroxyethoxy)benzophenone; and N,N-di-(2-ethylhexyl)-4-aminobenzoic acid ester of 4-(2-hydroxyethoxy)dibenzoylmethane.

The composition may also include one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide), their isomers, esters, salts and derivatives, and mixtures thereof.

The composition can also include one or more tanning actives. A suitable tanning active is dihydroxyacetone.

The composition can also include one or more anti-acne actives. Suitable anti-acne actives include resorcinol, sulfur, salicylic acid, erythromycin, benzoyl peroxide, and zinc. Additional anti-acne actives are described in U.S. Pat. No. 5,607,980 to McAtee et al., including 5-octanoyl salicylate; retinoic acid, N-acetyl-L-cysteine; octopirox; tetracycline; 2,4′,4′-trichloro-2′-hydroxy diphenyl ether; 3′ 4′,4′-trichlorocarbanilide; azelaic acid; derivatives of azelaic acid; phenoxyethanol; phenoxypropanol; phenoxyisopropanol; ethyl acetate; clindamycin; meclocycline; flavonoids; scymnol sulfate; deoxycholate; and cholate.

The composition can also include one or more desquamation actives. Suitable desquamation actives include sulfhydryl compounds, salicylic acid, and zwitterionic surfactants, including cetyl betaine.

The composition can also include one or more anti-cellulite actives. Suitable anti-cellulite actives include but are not limited to xanthine compounds such as caffeine, theophylline, theobromine, and aminophylline.

The composition can also include one or more chelating agents. Suitable chelating agents include furildioxime, furilmonoxime, and derivatives thereof.

The composition can also include one or more topical anesthetics. Suitable topical anesthetics include but are not limited to benzocaine, lidocaine, bupivacaine, chloroprocaine, dibucaine, etidocaine, mepivacaine, tetracaine, dyclonine, hexylcaine, procaine, and pramoxine.

The composition can also include one or more anti-wrinkle actives or anti-atrophy actives. Suitable anti-wrinkle actives or anti-atrophy actives include but are not limited to sulfur-containing D- and L-amino acids and their derivatives and salts, particularly the N-acetyl derivatives, a preferred example of which is N-acetyl-L-cysteine; thiols, e.g. ethanethiol; hydroxyacids (e.g., α-hydroxyacids such as lactic acid or glycolic acid and β-hydroxyacids such as salicylic acid or derivatives of salicylic acid such as octanoyl derivatives), keto acids (e.g., pyruvic acid), ascorbic acid (vitamin C), phytic acid, lipoic acid; lysophosphatidic acid, skin peel agents (e.g., phenol and the like), flavonoids (e.g., flavanones, chalcones, isoflavones, flavones), stilbenes, cinnamates, resveratrol, kinetin, zeatin, dimethylaminoethanol, peptides from natural sources (e.g., soy peptides), salts of sugar acids (e.g., magnesium gluconate), terpene alcohols (e.g., farnesol, geraniol, phytantriol), vitamin B compounds (e.g., thiamine (vitamin B1), pantothenic acid (vitamin B5), carnitine (vitamin Bt), riboflavin (vitamin B2), cobalamine (vitamin B12), pangamic acid or diisopropylamine dichloroacetate (vitamin B15), and their derivatives and salts.

The composition can also include one or more skin lightening agents. Suitable skin lightening agents include, but are not limited to, kojic acid, arbutin, tranexamic acid, ascorbic acid and derivatives (e.g., magnesium ascorbyl phosphate, sodium ascorbyl phosphate, other salts of ascorbyl phosphate, or ascorbyl glucoside), undecylenoyl phenylalanine, aloesin, and compounds disclosed in PCT Patent Application Publication No. WO 95/34280 by Hillebrand, including thioglycolic acid, cysteine, homocysteine, glutathione, thioglycerol, thiomalic acid, 2-mercaptopropionic acid, 3-mercaptopropionic acid, thiodiglycol, 2-mercaptoethanol, dithiothreitol, thioxanthene, thiosalicylic acid, thiolactic acid, thiopropionic acid, thiodiglycolic acid, N-acetyl-L-cysteine, and lipoic acid, as well as cosmetically- or pharmaceutically-acceptable salts thereof, as described in U.S. Pat. No. 5,296,500 by Hillebrand.

The composition can also include one or more antimicrobial or antifungal agents. Suitable antimicrobial and antifungal agents include B-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorocarbanilide, phenoxyethanol, phenoxypropanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lincomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione, and clotrimazole.

The composition can also include one or more particulate materials. Particulate materials include colored and uncolored pigments, interference pigments (nonlimiting examples include mica, layered with about 50-300 nm films of TiO₂, Fe₂O₃ silica, tin oxide, Cr₂O₃, and mixtures thereof; spherical TiO₂ particles having a size of from about 100 to about 300 nanometers; or alternatively, spherical TiO₂ particles having a size of from about 1 to about 30 micrometers; and mixtures thereof), inorganic powders (for example, iron oxides, ferric ammonium ferrocyanide, manganese violet, ultramarine blue, and chrome oxide), organic powders (for example, phthalocyanine blue and green pigment), composite powders, optical brightener particles, and combinations thereof. These particulates can, for instance, be platelet shaped, spherical, elongated or needle-shaped, or irregularly shaped; surface coated or uncoated; porous or non-porous; charged or uncharged.

The composition can include one or more flavonoids or derivatives thereof. The flavonoid can be synthetic materials or obtained as extracts from natural sources, which also further may be derivatized. Suitable flavonoids include, but are not limited to, unsubstituted flavanones; monosubstituted flavanones including methoxy flavanones; unsubstituted chalcones (especially the trans isomer); 2′,4-dihydroxy chalcone; mono-substituted chalcones; di-substituted chalcones; tri-substituted chalcones; glycosyl derivatives of chalcones; unsubstituted flavones; mono-substituted flavones; di-substituted flavones; unsubstituted coumarins; mono-substituted coumarins; di-substituted coumarins; unsubstituted chromones; mono-substituted chromones; di-substituted chromones; dicoumarols; chromanones; chromanols; and isoflavones. By the term “substituted” as used herein means flavonoids wherein one or more hydrogen atom of the flavonoid has been independently replaced with hydroxyl, C₁-C₈ alkyl, C₁-C₄ alkoxyl, 0-glycoside, and the like or a mixture of such substituents. Examples of suitable flavonoids include, but are not limited to, 2′-hydroxy flavanone, 6-hydroxy flavanone, 7-hydroxy flavanone, 5-methoxy flavanone, 6-methoxy flavanone, 7methoxy flavanone, 4′-methoxy flavanone, 2′-hydroxy chalcone, 4′-hydroxy chalcone, 2′,4-dihydroxy chalcone, 2′,4′-dihydroxy chalcone, 2,2′-dihydroxy chalcone, 2′,3-dihydroxy chalcone, 2′,5′-dihydroxy chalcone, 2′,3′,4′-trihydroxy chalcone, 4,2′,4′-trihydroxy chalcone, 2,2′,4′-trihydroxy chalcone, 7,2′-dihydroxy flavone, 3′,4′-dihydroxy naphthoflavone, 4′-hydroxy flavone, 5,6-benzoflavone, 7,8-benzoflavone, 5,7-dihydroxy-4′-methoxy isoflavone, 4-hydroxy coumarin, 7-hydroxy coumarin, 6-hydroxy-4-methyl coumarin, 3-formyl chromone, and 3-formyl-6-isopropyl chromone. Other examples of suitable flavonoids include flavanones such as hesperidin and glucosyl hesperidin, isoflavones such as soy isoflavones, including but not limited to apigenin, genistein, apigenenin, genistein, genistin, 6″-O-malonylgenistin, 6″-O-acetylgenistin, daidzein, daidzin, 6″-O-malonyldaidzin, 6″-O-acetylgenistin, glycitein, glycitin, 6″-O-malonylglycitin, and 6-O-acetylglycitin, and equol, their glucosyl derivatives, and mixtures thereof.

The composition can include one or more non-vitamin antioxidants and radical scavengers. Suitable non-vitamin antioxidants and radical scavengers include, but are not limited to, butylated hydroxytoluene, L-ergothioneine, tetrahydrocurcumin, cetyl pyridinium chloride, carnosine, diethylhexyl syrinylidene malonate, butylated hydroxybenzoic acids, 6-hydroxy-2,5,7,8-tetramethylchroman-2-carboxylic acid, gallic acid and its alkyl esters, including propyl gallate, uric acid and its salts and alkyl esters, sorbic acid and its salts, amines (e.g., N,N-diethylhydroxylamine and aminoguanidine), sulfhydryl compounds (e.g., glutathione), lysine pidolate, arginine pidolate, nordihydroguaretic acid, curcumin, lysine, methionine, proline, superoxide dismutase, melanin, and ubiquinone.

The composition can include one or more hair growth regulators. Suitable hair growth regulators include, but are not limited to, hexamidine, butylated hydroxytoluene (BHT), hexanediol, panthenol and pantothenic acid derivatives, their isomers, salts and derivatives, and mixtures thereof.

The composition can include one or more plant sterols or plant hormones. Suitable plant sterols or plant hormones include, but are not limited to, sitosterol, stigmasterol, campesterol, brassicasterol, kinetin, zeatin, and mixtures thereof.

The composition can include one or more protease inhibitors. Suitable protease inhibitors include, but are not limited to, hexamidine, vanillin acetate, menthyl anthranilate, and mixtures thereof.

The composition can include one or more tyrosinase inhibitors. Suitable tyrosine inhibitors include, but are not limited to, sinablanca (mustard seed extract), tetrahydrocurcumin, cetyl pyridinium chloride, and mixtures thereof.

The composition can include one or more anti-inflammatory agents. Suitable anti-inflammatory agents include, but are not limited to, the following categories of anti-inflammatory agents: (i) corticosteroids such as hydrocortisone, hydroxytriamcinolone, α-methyl dexamethasone, dexamethasone phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, and triamcinolone; (ii) non-steroidal anti-inflammatory agents such as: (i) oxicams such as piroxicam, isoxicam, tenoxicam, sudoxicam, and 4-hydroxy-2-methyl-N-phenyl-2H-1,2-benzothiazixine-3-carboxamide-1,1-dioxide (CP-14,304); (ii) salicylates, such as aspirin, disalcid, benorylate, trilisate, safapryn, solprin, diflunisal, and fendosal; (iii) acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; (iv) fenamates, such as mefenamic acid, meclofenamic acid, flufenamic acid, niflumic acid, tolfenamic acid, and etofenamate, (v) propionic acid derivatives such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic acid; (vi) pyrazoles such as phenylbutazone, oxyphenbutazone, feprazone, azapropazone, and trimethazone; and (vii) natural product derivatives such as a-bisabolol, aloe vera, chamomile, allantoin, glycyrrhizic acid, glycyrrhetic acid, esters of glycyrrhizic acid or glycyrrhetenic acid, including C₂-C₂₄ saturated or unsaturated esters, preferably C₁₀-C₂₄ esters, more preferably C₁₆-C₂₄ esters such as stearyl glycyrrhetinate, monoammonium glycyrrhizinate, monopotassium glycyrrhizinate, dipotassium glycyrrhizinate, 1-β-glycyrrhetic acid, 3-stearyloxy-glycyrrhetinic acid, disodium 3-succinyloxy-beta-glycyrrhetinate, candelilla wax, and combinations thereof.

The composition can include a skin soothing or skin healing agent. Suitable skin soothing or skin healing agents include, but are not limited to, panthenoic acid derivatives (including panthenol, dexpanthenol, ethyl panthenol), aloe vera, allantoin, bisabolol, and dipotassium glycyrrhizinate.

The composition can include a conditioning agent. The conditioning agent can be a humectant, a moisturizer, or a skin conditioner. Suitable conditioning agents include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose); hyaluronic acid; lactamide monoethanolamine; acetamide monoethanolamine; panthenol; allantoin; and mixtures thereof; propoxylated glycols described in U.S. Pat. No. 4,976,953 to On et al.; C₁-C₃₀ monoesters and polyesters of sugars and sugar derivatives, derived from a sugar or polyol moiety and one or more carboxylic acid moieties.

The composition can include a N-acyl amino acid or derivative thereof. Suitable N-acyl amino acids include, but are not limited to, N-acyl phenylalanine, N-acyl tyrosine, their isomers, including their D and L isomers, salts, derivatives, and mixtures thereof. An example of a suitable N-acyl amino acid derivative is N-undecylenoyl-L-phenylalanine.

The composition can include an anti-microbial active or antifungal active. Suitable anti-microbial or antifungal actives include β-lactam drugs, quinolone drugs, ciprofloxacin, norfloxacin, tetracycline, erythromycin, am ikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione, and clotrimazole.

Other alternatives for skin care actives can include, but are not limited to, dehydroepiandrosterone (DHEA), its analogs and derivatives; arbutin; dimethyl aminoethanol (DMAE); kojic acid; dihydroxyacetone (DHA); soy proteins and peptides (for example, protease inhibitors such as soybean trypsin inhibitor and Bowman-Birk inhibitor).

In skin care agents or cosmeceuticals as described above that are organic molecules or include an organic molecule component, saturated carbon atoms that are included within such organic molecules can be optionally substituted. In general, for such saturated carbon atoms, the following substituents can be employed: C₆-C₁₀ aryl; heteroaryl containing 1-4 heteroatoms selected from N, O, and S; C₁-C₁₀ alkyl; C₁-C₁₀ alkoxy; cycloalkyl; F; amino (NR¹R²); nitro; —SR; —S(O)R; —S(O₂)R; —S(O₂)NR¹R²; and —CONR¹R², which can in turn be optionally substituted. Further descriptions of additional potential optional substituents are provided below. Optional substituents as described above that are within the scope of the present invention are those that do not substantially affect the activity of the skin care agent or the cosmeceutical or the stability of the skin care agent or cosmeceutical, particularly the stability of the skin care agent or cosmeceutical in aqueous solution. Additionally, optional substituents as described above that are within the scope of the present invention are compatible with other ingredients included in a composition according to the present invention.

Definitions for a number of common groups that can be used as optional substituents are provided below; however, the omission of any group from these definitions cannot be taken to mean that such a group cannot be used as an optional substituent as long as the chemical and cosmeceutical requirements for an optional substituent are satisfied.

As used herein, the term “alkyl” refers to an unbranched, branched, or cyclic saturated hydrocarbyl residue, or a combination thereof, of from 1 to 12 carbon atoms that can be optionally substituted; the alkyl residues contain only C and H when unsubstituted. Typically, the unbranched or branched saturated hydrocarbyl residue is from 1 to 6 carbon atoms, which is referred to herein as “lower alkyl.” When the alkyl residue is cyclic and includes a ring, it is understood that the hydrocarbyl residue includes at least three carbon atoms, which is the minimum number to form a ring. Examples of alkyl groups include, but are not limited to, methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, and 1-octyl. As used herein, the term “alkenyl” refers to an unbranched, branched or cyclic hydrocarbyl residue having one or more carbon-carbon double bonds. As used herein, the term “alkynyl” refers to an unbranched, branched, or cyclic hydrocarbyl residue having one or more carbon-carbon triple bonds; the residue can also include one or more double bonds. With respect to the use of “alkenyl” or “alkynyl,” the presence of multiple double bonds cannot produce an aromatic ring. As used herein, the terms “hydroxyalkyl,” “hydroxyalkenyl,” and “hydroxyalkynyl,” respectively, refer to an alkyl, alkenyl, or alkynyl group including one or more hydroxyl groups as substituents; as detailed below, further substituents can be optionally included. As used herein, the term “aryl” refers to a monocyclic or fused bicyclic moiety having the well-known characteristics of aromaticity; examples include phenyl and naphthyl, which can be optionally substituted. As used herein, the term “hydroxyaryl” refers to an aryl group including one or more hydroxyl groups as substituents; as further detailed below, further substituents can be optionally included. As used herein, the term “heteroaryl” refers to monocyclic or fused bicyclic ring systems that have the characteristics of aromaticity and include one or more heteroatoms selected from O, S, and N. The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition. This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation. As used herein, the term “hydroxyheteroaryl” refers to a heteroaryl group including one or more hydroxyl groups as substituents; as further detailed below, further substituents can be optionally included. As used herein, the terms “haloaryl” and “haloheteroaryl” refer to aryl and heteroaryl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. The term “heterocycloalkyl” denotes a monocyclic or bicyclic carbocyclic moiety containing from 3 to 10 ring members, interrupted with one or more heteroatoms, which may be identical or different, selected from oxygen, nitrogen or sulfur atoms; for example, morpholinyl, thiomorpholinyl, homomorpholinyl, aziridyl, azetidyl, piperazinyl, piperidyl, homopiperazinyl, pyrrolidinyl, imidazolidinyl, pyrazolidinyl, tetrahydrofuryl, tetrahydrothienyl, tetrahydropyran, oxodihydropyridazinyl, or oxetanyl moieties, all of these moieties can be optionally substituted. As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents.

Substituent groups useful for substituting saturated carbon atoms in the specified group, moiety, or radical include, but are not limited to, —Z^{a}, ═O, —OZ^{b}, —SZ^{b}, ═S⁻, —NZ^{c}Z^{c}, ═NZ^{b}, ═N—OZ^{b}, trihalomethyl, —CF₃, —CN, —OCN, —SCN, —NO, —NO₂, ═N₂, —N₃, —S(O)₂Z^{b}, —S(O)₂NZ^{b}, —S(O₂)O⁻, —S(O₂)OZ^{b}, —OS(O₂)OZ^{b}, —OS(O₂)O⁻, —OS(O₂)OZ^{b}, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)Z^{b}, —NZ^{b}C(S)Z^{b}, —NZ^{b}C(O)O⁻, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z^{c} is independently Z^{b} or, alternatively, the two Z^{c}'s may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, —NZ^{c}Z^{c} is meant to include —NH₂, —NH-alkyl, —N-pyrrolidinyl, and —N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and —CH₂—CH₂—C(O)—CH₃, but is not limited to those specific alternatives and includes other alternatives known in the art. The one or more substituent groups, together with the atoms to which they are bonded, may form a cyclic ring, including, but not limited to, cycloalkyl and cycloheteroalkyl. Similarly, substituent groups useful for substituting unsaturated carbon atoms in the specified group, moiety, or radical include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —N₃, —S(O)₂Z^{b}, —S(O₂)O—, —S(O₂)OZ^{b}, —OS(O₂)OZ^{b}, —OS(O₂)O—, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

Similarly, substituent groups useful for substituting nitrogen atoms in heteroalkyl and cycloheteroalkyl groups include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —S(O)₂Z^{b}, —S(O₂)O⁻, —S(O₂)OZ^{b}, —OS(O₂)OZ^{b}, —OS(O₂)O—, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other alternatives for stereoisomers) as well as mixtures of stereoisomers in varying degrees of chiral purity or percentage of E and Z, including racemic mixtures, mixtures of diastereomers, and mixtures of E and Z isomers, unless specific stereoisomers or enantiomers are specifically excluded herein. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures, unless specific stereoisomers or enantiomers are specifically excluded herein. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures, unless specific stereoisomers or enantiomers are specifically excluded herein. It also encompasses the various diastereomers unless specifically excluded herein. Other structures may appear to depict a specific isomer, but that is merely for convenience, and is not intended to limit the invention to the depicted isomer. When the chemical name does not specify the isomeric form of the compound, it denotes any one of the possible isomeric forms or mixtures of those isomeric forms of the compound, unless any specific isomeric form or mixture of isomeric forms is excluded herein.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium; the equilibrium may strongly favor one of the tautomers, depending on stability considerations. For example, ketone and enol are two tautomeric forms of one compound.

In some alternatives, organic compounds that are skin care agents or cosmeceuticals or components of skin care agents or cosmeceuticals are in the form of a solvate. As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hemihydrate, monohydrate, dihydrate, trihydrate, hexahydrate, and other water-containing species. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

As used herein, the term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolyzable esters of the present compounds are the compounds whose carboxyls are present in the form of hydrolyzable ester groups. That is, these esters are pharmaceutically acceptable and can be hydrolyzed to the corresponding carboxyl acid in vivo.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁₀ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1 to 3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but is non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C₇-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the substituents are not otherwise described.

“Amino” as used herein refers to —NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising at least one heteroatom. The carbocyclyl can be fully saturated or partially saturated, but non-aromatic. For example, the carbocyclyl encompasses cycloalkyl. The carbocyclic and heterocyclic structures encompass compounds having monocyclic, bicyclic or multiple ring systems; and such systems may mix aromatic, heterocyclic, and carbocyclic rings. Mixed ring systems are described according to the ring that is attached to the rest of the compound being described.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent.

As used herein, the term “sulfamoyl” refers to a substituent with the structure —S(O₂)NH₂, wherein the nitrogen of the NH₂ portion of the group can be optionally substituted as described above.

As used herein, the term “carboxyl” refers to a group of the structure —C(O₂)H.

As used herein, the term “carbamyl” refers to a group of the structure —C(O₂)NH₂, wherein the nitrogen of the NH₂ portion of the group can be optionally substituted as described above; such an optional substitution replaces one or both hydrogens of the NH₂ moiety.

As used herein, the terms “monoalkylaminoalkyl” and “dialkylaminoalkyl” refer to groups of the structure -Alk₁-NH-Alk₂ and -Alk₁-N(Alk₂)(Alk₃), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above. The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

Other combinations of substituents are known in the art and, are described, for example, in U.S. Pat. No. 8,344,162 to Jung et al. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

In another alternative, the composition can include two or more skin care agents or agents that are cosmeceutical agents. Suitable combinations include, but are not limited, to, two or more skin care agents, two or more cosmeceuticals, and one or more skin care agents and one or more cosmeceuticals. In some alternatives, when two or more skin care agents or cosmeceuticals are present in a composition according to the present invention, the two or more skin care agents or cosmeceuticals can be linked to the same intermediate release linker. In other alternatives, when two or more skin care agents or cosmeceuticals are present in a composition according to the present invention, the two or more skin care agents or cosmeceuticals can be linked to different intermediate release linkers. When two or more different intermediate release linkers are used in the composition, they can be linked either to the same targeting moiety or to different targeting moities.

Typically, the intermediate release linker of the composition is a polymer. The polymer can be a protein or non-protein polymer. If the polymer is a protein polymer, it can be selected from the group consisting of albumin, gelatin, keyhole limpet hemocyanin, ferritin, and ovalbumin, and derivatives thereof. Typically, the protein polymer is albumin or gelatin, such as bovine serum albumin. The protein polymer can also be a synthetic polypeptide. The protein polymer can be pegylated. Typically, the intermediate release linker does not interact with the skin care agent or cosmeceutical agent and does not bind to or otherwise interact with the targeting moiety. If the polymer is a non-protein polymer, it can be selected from the group consisting of polyethylene glycol and polypropylene glycol. Typically, the non-protein polymer is polyethylene glycol.

The linkages between the skin care agent or cosmeceutical agent and the intermediate release linker and between the intermediate release linker and the targeting moiety can be covalent linkages or non-covalent linkages. In one alternative, the linkages are peptide linkages formed by derivatization of the components involved with peptides and the formation of a peptide linkage between the peptides. If the linkages are non-covalent linkages, they can be, for example, biotin/avidin or biotin/streptavidin linkages or specific antigen/antibody or hapten/antibody linkages.

The targeting composition can bind to native collagen fibers. In some cases, the native collagen fibers may differ from other collagen fibers in an organism that can be targeted by virtue of having their surfaces exposed as a consequence of the metabolic activity associated with processes such as inflammation.

The intermediate release linker can be stabilized by crosslinking, such as by reaction with an aldehyde, or by a reaction catalyzed by a transglutaminase, in which case the intermediate release linker includes groups that are substrates for a transglutaminase. In another alternative, the intermediate release linker can include a thiol-containing amino acid sequence derived from keratin or a biosynthesized thiol-containing amino acid sequence mimicking the properties of the thiol-containing amino acid sequence derived from keratin, or can include a hydrophobic amino acid sequence derived from elastin or a biosynthesized hydrophobic amino acid sequence mimicking the properties of the hydrophobic amino acid sequence derived from elastin.

Inflammation can lead to “uncoating” of the collagen fibers. MMPs, a family of zinc-dependent neutral endopeptidases, play a significant role in this connection. In addition many metabolic processes are known to be associated with inflammation, including new collagen deposition and turnover. We propose to use peptide sequences present in Von Willebrand's factor, which we have shown are able to cause polypeptide growth factors to bind tightly to collagen.

Collagen fibers are major constituents of tissue parenchyma or stroma that surround all cells. Such fibers contribute to the structural and functional properties of the majority of tissues. These fibers are normally not visible to cells or in direct contact with them as they are coated with a layer of proteoglycans, another major component of the connective tissues. This “coating” of the collagen fibers plays a key physiological role, since exposed collagen serves as a site for platelet attachment, and can initiate the blood clotting process. If collagen were exposed, abnormal hemostasis (blood clotting) would occur at multiple sites. At sites of inflammation or other processes associated with collagen remodeling, enhanced enzymatic activity can degrade this protective coat, thus exposing collagen. This now allows it to become a target for recognition. This can be used to deliver skin care agents or cosmeceuticals.

Collagen fibers are major constituents of tissue parenchyma. There are now over 30 distinct collagens. The first unique and distinct mammalian collagen, now known as type II collagen, was unique because it was not constructed from three identical polypeptides; rather, it was constructed of two identical polypeptides and one polypeptide that was slightly different (Strawich and Nimni, 1971). All these collagens have a characteristic repeating motif or a variation of this motif, typically a Gly-Pro-Hypro-Gly sequence, where Hypro is hydroxylated proline. Hydroxylated proline is not directly incorporated into the collagen molecule during polypeptide synthesis, but is produced by post-translational modification. Most important every fourth residue is by necessity glycine. Intervening amino acids can vary. The collagen molecules organize into a three-dimensional structure, leading to fibers. As mentioned collagen fibers are not normally directly accessible to cells as these fibers are coated with a layer of proteins and proteoglycans. This has an important physiological function as it prevents, among other things, platelets to attach and initiate the clotting cascade. It is only during the process of tissue damage (wound healing, release of inflammatory cytokines, and other processes related to collagen remodeling, including, but not limited to, skin damage) that metalloproteases and other related enzymes are released and remove such a coat, thereby exposing the surface of the collagen fibers.

Our working hypothesis, to which we are not bound, is that if we link a skin care agent or cosmeceutical to a peptide or peptides which recognize such naked collagen, the peptide or peptides will target such a site. As discussed below, such a peptide can be a decapeptide sequence, identical or similar to the sequence present in von Willebrand's factor (VWF) used by platelets to attach to collagen, which can be used to generate fusion proteins or other proteins or polypeptides which have an ability to strongly bind to collagen.

Since the VWF collagen binding domain was first identified, many new collagen binding sites of platelet collagen binding receptors, such as integrin α2β1, glycoprotein VI, and others, as well as more effective modifications of the VWF collagen binding domain, are being constantly mapped. These can provide binding sequences of increased affinity and specificity that can be incorporated into targeting compositions for delivery of a cosmeceutical or skin care agent according to the present invention. Accordingly, the targeting composition can comprise a targeting moiety, as discussed further below, that is a collagen binding site of a platelet collagen binding receptor, including, but not limited to, integrin α2β1 and glycoprotein VI.

One improvement specific to this application is to target sites of the skin that are susceptible to treatment by skin care or cosmeceutical agents by focusing on identifiable specific changes that occur in immediate proximity to these sites; in many cases, these specific changes are associated with skin damage, which may include, but are not necessarily limited to, skin damage associated with aging, solar exposure, inflammation, infection, or other causes. For this purpose we have selected a unique event that occurs at these locations, the exposure of normally masked collagen fibers, which become visible to targets as a result of the active metabolic activity associated with inflammation or other insults or damage affecting skin. We believe that this approach provides enhanced targeting by orders of magnitude to areas of the skin requiring such treatment as compared to the passive targeting concept. Typically, the native collagen fibers to which a targeting composition according to the present invention is bound differ from other collagen fibers in the organism as they are clearly recognizable to the targeting moiety, by virtue of having their surface exposed as a consequence of the metabolic activity associated with inflammation or other insults or damage.

One particular aspect of this invention, therefore, is a focus on the synergistic role of local inflammation. Such local inflammation is associated with recognizable exposure of local collagen fibers and is also associated with skin damage, such as that caused by aging, solar exposure, or other causes.

Accordingly, one advantage of the use of the targeting methods of the present invention, employing the linkage of a skin care agent or cosmeceutical to a peptide sequence targeting collagen, is that they have the ability to deliver a wide variety of skin care agents or cosmeceuticals, including but not limited to the skin care agents or cosmeceuticals described above.

In some alternatives, as discussed below, it may be desirable to insert multiple binding motifs on the surface of the targeting particle to assure good linking and to stabilize its attachment. On the other hand it is possible that protruding PEG chains may suffice to achieve this goal. In one alternative, the protruding peptides can be extended by inserting repeating sequences of glycine. Glycine provides maximum rotation around peptide bonds, and therefore maximal degree of motion. When such polyglycine extensions are employed, the polyglycine extensions typically range up to 50 repeating units. The displacement between the most adjacent binding sites among parallel oriented molecules is in the range of 2-4 nm. Free random movement of the glycine chains and the generation of as many attachments as possible are desirable. In another alternative, the extensions can be made more rigid, such as by using repeating Gly-Pro-Pro-Gly sequences, to generate a collagen like rigid triple helical extension radiating from the surface of the targeting particle.

Other alternatives employing compositions according to the present invention are also within the scope of the present invention. In one such alternative, two separate preparations with different targeting sites are administered to the same organism in need of skin care treatment. In another such alternative, known as pretargeting, a secondary targeting reagent that specifically binds to the targeting composition and directly targets the area of the skin to be treated, such as a suitable antibody, which can be a monoclonal antibody, is administered first. In one alternative, the monoclonal antibody or other secondary targeting reagent is conjugated to one of two binding partners that use the biotin-avidin link, while the targeting composition, including the skin care agent or cosmeceutical, is conjugated to the other of the two binding partners that use the biotin-avidin link. In another alternative, the monoclonal antibody or other secondary targeting reagent and the targeting composition are both conjugated to biotin or a derivative or analogue of biotin, and a biotin-binding component is introduced to cross-link the monoclonal antibody or other secondary targeting reagent to the targeting composition. The avidin-binding component can be selected from the group consisting of avidin, streptavidin, a derivative or analogue of avidin or streptavidin, and a biotin-binding antibody.

In still another approach, targeting compositions according to the present invention are used that include at least two antibodies, wherein each antibody is an antibody for a specific receptor on the surface of cells in the organism to be treated by administration of a skin care product or cosmeceutical, the receptors occurring in the same cell to be treated.

In yet another approach according to the present invention, a targeting composition according to the present invention has two functionalities in addition to the targeting moiety described above. These two functionalities are: (1) a binding functionality to a noninternalizing receptor on the surface of a cell to be treated by administration of a skin care agent or cosmeceutical; and (2) an initially hidden functionality through a cell-penetrating peptide that is activated only after binding to the cell surface at slightly acidic conditions in the interior of the cell to be treated.

However, in many alternatives according to the present invention, it is not required for active agents present in skin care products or cosmeceuticals to bind to receptors to cells to be treated or internalize within cells to be treated.

In one alternative, one aspect of present invention is a targeting composition comprising: (1) a skin care agent or cosmeceutical; (2) an intermediate release linker bound to the therapeutic agent; and (3) a targeting moiety bound to the intermediate release linker as described further below for binding the targeting composition to native collagen fibers, such as a peptide motif identical or similar to that used by von Willebrand's factor to bind to collagen. The ingredients of this composition and the methods used to link them in the composition are described further below.

As indicated above, one of a number of peptide motifs can be used for binding the composition to native collagen fibers. Typically, such a peptide motif is based on the peptide motif used by von Willebrand's factor to bind to collagen.

Such sequences include, but are not limited to: (1) Trp-Arg-Glu-Pro-Ser-Phe-Met-Ala-Leu-Ser (WREPSFMALS) (SEQ ID NO: 1); (2) Trp-Arg-Glu-Pro-Ser-Phe-Cys-Ala-Leu-Ser (WREPSFCALS) (SEQ ID NO: 2); (3) peptides related to (1) or (2) by one or more conservative amino acid substitutions, as defined below, including, but not limited to: (3a) Trp-Arg-Asp-Pro-Ser-Phe-Met-Ala-Leu-Ser (WRDPSFMALS) (SEQ ID NO: 3); (3b) Trp-Arg-Asp-Pro-Ser-Phe-Cys-Ala-Leu-Ser (WRDPSFCALS) (SEQ ID NO: 4); (3c) Trp-Arg-Glu-Pro-Ser-Phe-Met-Ala-Ile-Ser (WREPSFMAIS) (SEQ ID NO: 5); (3d) Trp-Arg-Glu-Pro-Ser-Phe-Cys-Ala-Ile-Ser (WREPSFCAIS) (SEQ ID NO: 6); (3e) Trp-Arg-Asp-Pro-Ser-Phe-Met-Ala-Ile-Ser (WRDPSFMAIS) (SEQ ID NO: 7); and (3f) Trp-Arg-Asp-Pro-Ser-Phe-Cys-Ala-Ile-Ser (WRDPSFCAIS) (SEQ ID NO: 8).

Conservative amino acid substitutions are well known in the art. More specifically, in a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g. Watson et al., Molecular Biology of the Gene, 4th Edition, 1987, Benjamin/Cummings, p. 224). In particular, such a conservative variant has a modified amino acid sequence, such that the change(s) do not substantially alter the protein's (the conservative variant's) secondary or tertiary structure and/or activity, specifically binding activity in this context. Conservative amino acid substitution generally involves substitutions of amino acids with residues having similar properties (e.g., acidic, basic, positively or negatively charged, polar or non-polar, or other similarities) such that the substitutions of even critical amino acids do not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gln); (4) arginine (R or Arg), lysine (K or Lys); (5) isoleucine (I or Ile), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. As another example, for some purposes, one may regard all non-polar amino acids as conservative substitutions for each other.

Additionally, such sequences include the decapeptides of SEQ ID NOs: 1-8 extended at both the amino-terminus and the carboxyl-terminus by the addition of the sequences Gly-Pro-Pro-Gly (GPPG). Accordingly, these sequences are as follows: (4) Gly-Pro-Pro-Gly-Trp-Arg-Glu-Pro-Ser-Phe-Met-Ala-Leu-Ser-Gly-Pro-Pro-Gly (GPPGWREPSFMALSGPPG) (SEQ ID NO: 9); (5) Gly-Pro-Pro-Gly-Trp-Arg-Glu-Pro-Ser-Phe-Cys-Ala-Leu-Ser-Gly-Pro-Pro-Gly (GPPGWREPSFCALSGPPG) (SEQ ID NO: 10); (6) Gly-Pro-Pro-Gly-Trp-Arg-Asp-Pro-Ser-Phe-Met-Ala-Leu-Ser-Gly-Pro-Pro-Gly (GPPGWRDPSFMALSGPPG) (SEQ ID NO: 11); (7) Gly-Pro-Pro-Gly-Trp-Arg-Asp-Pro-Ser-Phe-Cys-Ala-Leu-Ser-Gly-Pro-Pro-Gly (GPPGWRDPSFCALSGPPG) (SEQ ID NO: 12); (8) Gly-Pro-Pro-Gly-Trp-Arg-Glu-Pro-Ser-Phe-Met-Ala-Ile-Ser-Gly-Pro-Pro-Gly (GPPGWREPSFMAISGPPG) (SEQ ID NO: 13); (9) Gly-Pro-Pro-Gly-Trp-Arg-Glu-Pro-Ser-Phe-Cys-Ala-Ile-Ser-Gly-Pro-Pro-Gly (GPPGWREPSFCAISGPPG) (SEQ ID NO: 14); (10) Gly-Pro-Pro-Gly-Trp-Arg-Asp-Pro-Ser-Phe-Met-Ala-Ile-Ser-Gly-Pro-Pro-Gly (GPPGWRDPSFMAISGPPG) (SEQ ID NO: 15); and (11) Gly-Pro-Pro-Gly-Trp-Arg-Asp-Pro-Ser-Phe-Cys-Ala-Ile-Ser-Gly-Pro-Pro-Gly (GPPGWRDPSFCAISGPPG) (SEQ ID NO: 16).

In another alternative, peptide sequences SEQ ID NO: 1 through SEQ ID NO: 16 (i.e., SEQ ID NO: 1, SEQ ID NO: 2, SEQ ID NO: 3, SEQ ID NO: 4, SEQ ID NO: 5, SEQ ID NO.: 6, SEQ ID NO.: 7, SEQ ID NO.: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16) can be incorporated into an elongated peptide structure of Formula (I):

(I)
[Gly-Pro-Pro-Gly-X₁-Gly-Pro-Pro-Gly-X₂-Gly-Pro-
Pro-Gly]ₙ

wherein: (1) X₁ and X₂ are one of peptide sequences SEQ ID NO: 1 through SEQ ID NO: 16, described above; and (2) n is an integer from 1 to 15.

This repeating sequence would provide: (1) increased sites of attachment; and (2) an intervening sequence which resembles collagen. This sequence should increase the compatibility with the surface of collagen, since it is similar to the normally adjacent sequences which are normal constituents of the native collagen molecule.

Other such sequences which cover longer ranges, and therefore would increase interaction, can be used. Also, modified sequences that take into account the triple helical configuration of collagen can be designed to enhance the longer range contact and surface compatibility with the triple stranded configuration displayed on the surface of the fiber. In particular, peptide motifs that bind collagen with a binding affinity of at least 80% of the binding affinity of either SEQ ID NO: 1 or SEQ ID NO: 2 for collagen can be used and are encompassed by the invention. Preferably, such peptide motifs bind collagen with a binding affinity of at least 90% of the binding affinity of either SEQ ID NO: 1 or SEQ ID NO: 2 for collagen. More preferably, such peptide motifs bind collagen with a binding affinity of at least 95% of the binding affinity of either SEQ ID NO: 1 or SEQ ID NO: 2 for collagen. Still more preferably, such peptide motifs bind collagen with a binding affinity of at least 97.5% of the binding affinity of either SEQ ID NO: 1 or SEQ ID NO: 2 for collagen. Such sequences can be deduced from crystallography data or other techniques known in the art for determining protein-peptide interactions, including NMR.

The nature of this sequence can be readily determined by observing a collagen model which includes the individual collagen molecules packed into a three-dimensional quarter-staggered array.

Other polypeptide sequences known to bind to collagen can alternatively be used.

In one alternative, the targeting moiety can be a targeting moiety in which the peptide sequences WREPSFMALS (SEQ ID NO: 1) or WREPSFCALS (SEQ ID NO: 2) can be incorporated into a variety of molecules of diverse nature to generate polypeptides which range from 2,000 to 10,000 daltons in size. The flanking sequences will vary but in general will mimic sequences found in native proteins, primarily collagen or elastin, with various degrees of hydrophilicity and hydrophobicity. As used herein, the term “mimics” refers to a sequence that results in the peptide including the sequence specifically binding to at least one target or receptor that the native protein intended to be mimicked by the sequence specifically binds to with an affinity that is at least 50% of the affinity of the native protein for the target or receptor. Inserted amino acids containing reactive groups will allow for coupling to the skin care product or cosmeceutical. In its simplest form one, two, or three collagen binding domains are incorporated in a construct, separated by spacers with lead to various suitable conformations. These should be able to bridge the span between repeating domains on the surface of collagen, and therefore enhance the binding of the vector.

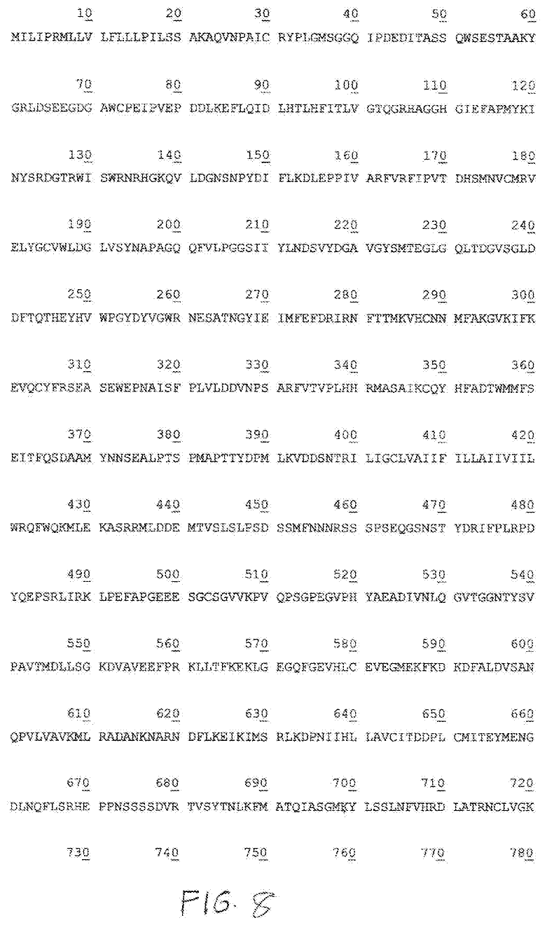

Multiple binding, to laterally displaced equivalent sites, on the surface of the collagen fiber should enhance binding affinity. Although the inventors do not intend to be bound by this theory, the inventors estimate a lateral displacement of about 3 nm, or twice the diameter of a collagen molecule to be in that range. Therefore spacers that maximally elongate in solution, i.e., alternating sequences (polar/nonpolar) which contribute to a β-like sheet or polylysine or polyglycine which stretch out in solution may give rise to adequate spacers (FIG. 7). This approach should provide (1) increased sites of attachment; and (2) an intervening sequence which resembles collagen.

In another alternative, the collagen binding domain sequences can be subject to pegylation (covalent conjugation with polyethylene glycol (PEG) moieties). These PEG polymers are nonionic, nontoxic, biocompatible, and highly hydrophilic. Their use provides increased solubility for hydrophobic therapeutic agents and increased bioavailability for skin cells. Such structures can be inserted at Site (C) in FIG. 7.

In yet another alternative, if a peptide is selected from an internal sequence of a protein, terminal amidation at the carboxyl-terminus or acetylation at the amino-terminus will eliminate the charge at the termini. In addition, these modifications will make the resulting peptide more stable towards enzymatic degradation by exopeptidases. Biotin and fluorescein isothiocyanate (FITC) are activated precursors used for fluorescein labelings. For efficient N-terminal labeling, a seven-atom aminohexanoyl spacer (NH₂—CH₂—CH₂—CH₂—CH₂—CH₂—COOH) can be inserted between the fluorophore (fluorescein) and the N-terminus of the peptide. One common means of conjugation involves the use of maleimide, which couples amino-terminal or carboxyl-terminal cysteine residues of the peptide to the carrier protein. Other conjugation methods are known in the art.

The decapeptides WREPSFMALS (SEQ ID NO: 1) or WREPSFCALS (SEQ ID NO: 2) involve a series of exposed amino acids, located strategically within the N-terminus, in an area extending from residues 570 (F) to 682 of Von Willebrand factor (Takagi, Asai et al. 1992). By binding competition this decapeptide was found to bind, on a molar basis, 20 times more efficiently to collagen than the intact VWF (Takagi, Asai et al. 1992). Further examination of the crystal structure of the collagen binding regions of VWF A-3 Domain (Ichikawa, Osawa et al. 2007); (Romijn, Westein et al. 2003); (Staelens, Hadders et al. 2006) as well as the complementary collagen exposed surface (Lisman, Raynal et al. 2006) is expected to yield collagen binding domains (CBDs) with an increased binding affinity.

Collagens are large, triple-helical proteins that form fibrils and network-like structures in the extracellular matrix. They have played a major role in the evolution of metazoans from their earliest origins. Cell adhesion receptors that interact with collagen, such as the integrins are at least as old as the collagens (Heino, Huhtala et al. 2009); (Whittaker and Hynes 2002) and instrumental in the evolution of bone, cartilage, and the immune system in chordates. In vertebrates collagen binding receptor tyrosine kinases send signals into cells after adhesion to collagen. Nevertheless, collagen continues to be seen primarily as an inert scaffold. To the inventors of the present application, the value of using it as a target became most relevant when they observed that it is only at sites of pathology or rapid tissue remodeling, including sites associated with skin care damage, that collagen fibers become devoid of their normal proteoglycan coating, and therefore recognizable as such.

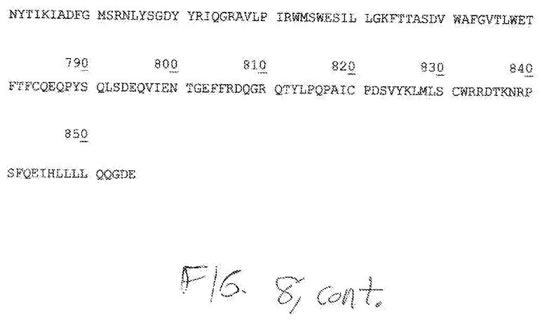

Other CBDs, such as the discoidin domain receptors, DDR1 and DDR2, are receptor tyrosine kinases known to be activated by native triple-helical collagen. The sequence on collagen that binds DDR2 with highest affinity has similarity to the binding site for von Willebrand's factor, GVMGFO (O is hydroxyproline) (SEQ ID NO: 17). (Konitsiotis, Raynal et al. 2008). The scattered amino acids on the binding site on the ligand are highlighted (FIG. 8). The complete DDR2 amino acid sequence (SEQ ID NO: 18) is shown in FIG. 8.

Accordingly, in compositions for delivery of a skin care agent or cosmeceutical according to the present invention, the CBDs from DDR1 and DDR2 can be employed. These include: (1) the native CBDs from DDR1 and DDR2; and (2) CBDs incorporating the amino acids on the surface of the three-dimensional protein structure of DDR1 or DDR2 in which at least one of the amino acids not directly contacting collagen is replaced with a conservative amino acid substitution as described above such that the CBD binds collagen with a binding affinity of at least 80% of the binding affinity of the native CBD from DDR1 or DDR2.

Yet another alternative for a CBD is the synthetic peptide P-15, which is a synthetic 15-residue peptide that binds to collagen at the single mammalian collagenase cleavage site. This peptide has the sequence GTPGPGGIAGQRGVV (SEQ ID NO: 19). The single unique collagenase site is particularly significant as it becomes exposed during periods of active collagen remodeling as occurs during fibrosis and metastasis. Additionally, the CBD can be a CBD derived from the sequence GTPGPGGIAGQRGVV (SEQ ID NO: 19) by one or more conservative amino acid substitutions such that the CBD binds collagen with a binding affinity of at least 80% of the binding affinity of the sequence GTPGPGGIAGQRGVV (SEQ ID NO: 19).

An increased number of CBDs can be employed, properly spaced from each other, as shown in FIG. 7. Peptide (B) of the array shown in FIG. 7 can be designed to match the profile of one or more components of the skin care agent or cosmeceutical being carried; and amino acid sequences can be inserted and crosslinking mechanisms can be adjusted to the hydrophobic or electrostatic character of the one or more components of the skin care agent or cosmeceutical being carried. Collagen sequences, especially if repeated, will encourage collagen-like folding. Suitable sequences can be generated as well as cyanogen bromide peptides by cleavage of the native collagen molecule (Deshmukh and Nimni 1973). Such peptides fold and generate small size stable triple helical structures (“mini-collagens”), thermodynamically favored at 37° C., which should enhance binding to the fibers.

Certain conservative amino acid substitutions, positive or negative, can improve binding affinity. Additionally, in another alternative, the CBDs can include one or more amino acids included in the collagen binding site for DDR2 and on the surface of DDR2 as shown in FIG. 8.

The present invention is designed to result in minimal toxicities that can be achieved as long as the skin care agent or cosmeceutical is not released from targeting compositions at sites other than those targeted by the composition as long as inactive prodrugs of skin care agents or cosmeceuticals included in the targeting composition are decomposed and removed from the body with minimal side effects. Compositions according to the present invention are intended to minimize such side effects, which can exist for conventionally applied skin care agents or cosmeceuticals, particularly when applied to the skin in greater than optimum quantities.

Suitable alternatives for skin care agents and cosmeceuticals that can be included in targeting compositions according to the present invention are as described above.

An important factor for one of ordinary skill in the art to consider in determining the construction of the targeting composition is the relative hydrophobicity or hydrophilicity of the skin care agent or cosmeceutical, including its solubility in water or aqueous solutions. This can assist one of ordinary skill in the art in determining suitable intermediate release linkers and targeting moieties as well as appropriate techniques for linking the skin care agent or cosmeceutical, the intermediate release linker, and the targeting moiety, including the reactive groups to be used; suitable combinations of reactive groups are described further below. This applies to all of the skin care agents or cosmeceuticals addressed above, including, but not limited to, anti-inflammatory agents.

When a targeting composition according to the present invention includes an antibody, as used herein, unless otherwise specified, the term “antibody” includes all antibody derivatives with appropriate binding specificity, including naturally occurring antibodies, monoclonal antibodies, chimeric antibodies, humanized antibodies, and single-chain antibodies such as sFv antibody derivatives. In some cases, the term “antibody” may also include antibody fragments with appropriate binding specificity, including such antibody fragments as sFv, Fv, Fab, Fab′ and F(ab)′₂ fragments.

The intermediate release linker that holds the skin care agent or cosmeceutical in place via ionic, covalent, or hydrophobic linkages can be further stabilized by a variety of techniques. These techniques can include various crosslinking modalities, some of which may offer various degrees of resistance to biodegradation. These crosslinking modalities can include the use of aldehydes such as formaldehyde to generate reversible crosslinks or glutaraldehyde to generate irreversible crosslinks. Alternatively, these crosslinks can be generated biologically through the activity of transglutaminases or other enzymes, which would require the insertion of suitable amino acid moieties into the primary structure of the intermediate release linker, such as free amino groups, free carboxyl groups, or combinations of such groups.

In another alternative, the composition can include cell-penetrating peptides and protein transcription-activating peptides, such as oligo-arginine and transcription activator peptides, to enable the internalization of agents that otherwise would not be taken up effectively by epidermal cells because of the lipophilic barrier generated by cell membranes of such cells.

Cell-penetrating peptides include, but are not limited to, the following alternatives.

One group of alternatives for cell-penetrating peptides are the cell-penetrating peptides disclosed in U.S. Pat. No. 7,754,678 to Guo et al., including RRHHCRSKAKRSRHH (SEQ ID NO: 20), SRRHHCRSKAKRSRHH (SEQ ID NO: 21), SARHHCRSKAKRSRHH (SEQ ID NO: 22), SRAHHCRSKAKRSRHH (SEQ ID NO: 23), SRRAHCRSKAKRSRHH (SEQ ID NO: 24), SRRHACRSKAKRSRHH (SEQ ID NO: 25), SRRHHARSKAKRSRHH (SEQ ID NO: 26), SRRHHCRAKAKRSRHH (SEQ ID NO: 27), SRRHHCRSAAKRSRHH (SEQ ID NO: 28), SRRHHCRSKAARSRHH (SEQ ID NO: 29), SRRHHCRSKAKASRHH (SEQ ID NO: 30), SRRHHCRSKAKRARHH (SEQ ID NO: 31), SRRHHCRSKAKRSAHH (SEQ ID NO: 32), RRHHCRSKAKRSR (SEQ ID NO: 33), RKGKHKRKKLP (SEQ ID NO: 34), GRKGKHKRKKLP (SEQ ID NO: 35), and GRRHHCRSKAKRSRHH (SEQ ID NO: 36).

Another group of alternatives for cell-penetrating peptides are the peptides disclosed in U.S. Pat. No. 7,709,606 to Jalinot et al., including NRKKRRQRRR (SEQ ID NO: 37), RRRRRRR (SEQ ID NO: 38), RRRRRRRR (SEQ ID NO: 39), and RRRRRRRRR (SEQ ID NO: 40).

Yet another group of alternatives for cell-penetrating peptides are the D-amino-acid containing peptides disclosed in U.S. Pat. No. 7,704,954 to Szeto et al., including Tyr-D-Arg-Phe-Lys-NH₂, 2′,6′-Dmt-D-Arg-Phe-Lys-NH₂, Phe-D-Arg-Phe-Lys-NH₂, D-Arg-2′,6′-Dmt-Lys-Phe-NH₂, and 2′,6′-Dmp-D-Arg-Phe-Lys-NH₂.

Yet another group of alternatives for cell penetrating peptides are the peptides disclosed in U.S. Pat. No. 7,579,318 to Divita et al., including GLX₉RAX₉RX₁LX₂RSLX₉X₃X₄X₅X₆X₇X₈ (SEQ ID NO: 41), wherein X₁ is selected from the group consisting of A, L, and G, X₂ is selected from the group consisting of W and a peptide bond, X₃ is selected from the group consisting of R and K, X₄ is selected from the group consisting of K, L, and S, X₅ is selected from the group consisting of L and K, X₆ is selected from the group consisting of R and W, X₇ is selected from the group consisting of K and S, X₈ is selected from the group consisting of A, V, and Q, and X₉ is selected from the group consisting of W, F, Y, and a non-amino-acid aromatic group. Additional non-peptide moieties can be covalently bound to this peptide sequence, in order to improve the overall stability of the molecule, and/or to provide it with additional properties, such as targeting ability. For example, a moiety such as cysteamide, a cysteine, a thiol, an amide, a carboxyl moiety, a linear or branched C₁₋₆ optionally substituted alkyl moiety, a primary or secondary amine, an osidic derivative, a lipid, a phospholipid, a fatty acid, a cholesterol, or a polyethylene glycol can be covalently linked to the carboxyl terminus of the peptide sequence. A moiety such as an acetyl moiety, a fatty acid moiety, a cholesterol moiety, or a polyethylene glycol can be covalently linked to the amino terminus of the peptide sequence. Additionally, in some alternatives, for example in the amino-terminal addition of cholesterol, a secondary peptide bridge can be used to bind a non-peptide molecule to the peptide sequence. Preferred examples of this alternative of the cell-penetrating peptide are

(SEQ ID NO: 42)
GLWRALWRLLRSLWRLLWKA,
(SEQ ID NO: 43)
GLWRALWRALWRSLWKLKRKV,
(SEQ ID NO: 44)
GLWRALWRALRSLWKLKRKV,
(SEQ ID NO: 45)
GLWRALWRGLRSLWKLKRKV,
(SEQ ID NO: 46)
GLWRALWRGLRSLWKKKRKV,
(SEQ ID NO: 47)
GLWRALWRLLRSLWRLLWKA,
(SEQ ID NO: 48)
GLWRALWRALWRSLWKLKWKV,
(SEQ ID NO: 49)
GLWRALWRALWRSLWKSKRKV,
(SEQ ID NO: 50)
GLWRALWRALWRSLWKKKRKV,
and
(SEQ ID NO: 51)
GLWRALWRLLRSLWRLLWSQ.

Yet another group of alternatives for cell-penetrating peptides are the peptides disclosed in U.S. Pat. No. 7,576,058 to Lin et al. These peptides include AAVALLPAVLLALLAPAAADQNQLMP (SEQ ID NO: 52) and AAVALLPAVLLALLAPAAANYKKPKLMP (SEQ ID NO: 53).

Other cell-penetrating peptides are known in the art.

Such cell-penetrating peptides are typically covalently bound to the targeting composition, or a component thereof, by reactions such as those described below. However, in some alternatives, the cell-penetrating peptides can be non-covalently bound to the targeting composition or a component thereof.

Transcription-activating peptides include, but are not limited, to peptides disclosed in U.S. Pat. No. 7,087,711 to Ptashne et al. These peptides include QLPPWL (SEQ ID NO: 54), QFLDAL (SEQ ID NO: 55), LDSFYV (SEQ ID NO:56), PPPPWP (SEQ ID NO: 57), SWFDVE (SEQ ID NO: 58), QLPDLF (SEQ ID NO: 59), PLPDLF (SEQ ID NO: 60), FESDDI (SEQ ID NO: 61), QYDLFP (SEQ ID NO: 62), LPDLIL (SEQ ID NO: 63), LPDFDP (SEQ ID NO: 64), LFPYSL (SEQ ID NO: 65), FDPFNQ (SEQ ID NO: 66), DFDVLL (SEQ ID NO: 67), HPPPPI (SEQ ID NO: 68), LPGCFF (SEQ ID NO: 69), QYDLFD (SEQ ID NO: 70), YPPPPF (SEQ ID NO: 71), PLPPFL (SEQ ID NO: 72), LPPPWL (SEQ ID NO: 73), VWPPAV (SEQ ID NO: 74), DPPWYL (SEQ ID NO: 75), LY (SEQ ID NO: 76), FDPFGL (SEQ ID NO: 77), PPSVNL (SEQ ID NO: 78), YLLPTCIP (SEQ ID NO: 79), LQVHNST (SEQ ID NO: 80), VLDFTPFL (SEQ ID NO: 81), HHAFYEIP (SEQ ID NO: 82), PWYPTPYL (SEQ ID NO: 83), YPLLPFLPY (SEQ ID NO: 84), YFLPLLST (SEQ ID NO: 85), FSPTFWAF (SEQ ID NO: 86), and LIMNWPTY (SEQ ID NO: 87).

Other transcription-activating peptides are known in the art.

Such transcription-activating peptides are typically covalently bound to the targeting composition, or a component thereof, by reactions such as those described below. However, in some alternatives, the transcription-activating peptides can be non-covalently bound to the targeting composition or a component thereof.

Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150, incorporated herein by this reference. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleimides can react with amino groups, such as the ε-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the ε-amino group of the side chain of lysine. Many other electrophilic reagents are known that will react with the ε-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G. T. Hermanson, “Bioconjugate Techniques” (Academic Press, San Diego, 1996), pp. 137-146, incorporated herein by this reference. Additionally, electrophilic reagents are known that will react with carboxylate side chains such as those of aspartate and glutamate, such as diazoalkanes and diazoacetyl compounds, carbonydilmidazole, and carbodiimides. These are described in G. T. Hermanson, “Bioconjugate Techniques” (Academic Press, San Diego, 1996), pp. 152-154, incorporated herein by this reference. Furthermore, electrophilic reagents are known that will react with hydroxyl groups such as those in the side chains of serine and threonine, including reactive haloalkane derivatives. These are described in G. T. Hermanson, “Bioconjugate Techniques,” (Academic Press, San Diego, 1996), pp. 154-158, incorporated herein by this reference. In another alternative embodiment, the relative positions of electrophile and nucleophile (i.e., a molecule reactive with an electrophile) are reversed so that, for example, if a protein is to be coupled to another molecule, the protein to be coupled has an amino acid residue with an electrophilic group that is reactive with a nucleophile and the molecule with which the protein to be coupled includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail.

Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiim ides. Hydroxyl groups can be reacted with epoxides, oxiranes, carbonyldiimidazole, N,N′-disuccinimidyl carbonate, N-hydroxysuccinimidyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. In some cases, it may be desirable to introduce a specific functional group that can subsequently be cross-linked. Such functional groups that can be introduced for cross-linking purposes can include, for example, sulfhydryl groups, carboxylate groups, primary amine groups, aldehyde groups, and hydrazide groups. Such cross-linking reagents and reactions, including the introduction of suitable functional groups for cross-linking, are described in G. T. Hermanson, “Bioconjugate Techniques” (Academic Press, San Diego, 1996), incorporated herein in its entirety by this reference.

The intermediate release linker of the composition is typically a polymer that shields the skin care agent or cosmeceutical of the composition from clearance by macrophages. The polymer can be a protein polymer or a non-protein polymer. If the polymer is a protein polymer, the protein polymer can be, but is not limited to, a protein such as albumin or gelatin. Other suitable proteins are known in the art and include, but are not limited to, keyhole limpet hemocyanin, ferritin, and ovalbumin. If albumin is used, it is typically bovine serum albumin, although analogous serum albumin proteins from other species, such as rats, mice, or horses, can also be used. As used herein, the term “protein” includes synthetic polypeptides including polypeptides of random sequence or defined sequence, block synthetic polypeptides that contain multiple regions, each region being comprised of residues of the same amino acid, or synthetic polypeptides of alternating sequence (i.e., polymers of a defined dipeptide or tripeptide); the synthetic polypeptides can be linear or branched, and many variations are possible. Typically, such synthetic polypeptides are produced by polymerization of α-amino acid-N-carboxyanhydrides (NCAs); the production and use of synthetic polypeptides are described in T. J. Deming, “Synthetic Polypeptides for Biomedical Applications,” Proq. Polymer Sci. 32: 858-875 (2007). Typically, if the intermediate release linker is a protein, it possesses at least one metalloprotease cleavage site for better local delivery, especially to epidermal cells, and less systemic toxicity. If the intermediate release linker is a protein, it can optionally be substituted with polyethylene glycol moieties (pegylation). When the protein intermediate release linker is pegylated, typically, the length of the polyethylene glycol chains, expressed in terms of the number of ethylene glycol monomers, is from about 10 ethylene glycol monomers to about 60 ethylene glycol monomers. Preferably, the length of the polyethylene glycol chains is from about 20 ethylene glycol monomers to about 40 ethylene glycol monomers. More preferably, the length of the polyethylene glycol chains is about 32 ethylene glycol monomers (i.e., polyethylene glycol with a molecular weight of about 2000 daltons). The polyethylene glycol chains can be attached through reactive groups, including reactive groups in side chains of amino acids in the polypeptide sequence of the protein, such as hydroxyl groups in serine or threonine or the ε-amino groups of lysine. Procedures for coupling polyethylene glycol groups to protein molecules are well known in the art; for example, and not by way of limitation, coupling can be performed by the creation of a reactive electrophilic intermediate that is capable of spontaneously coupling to nucleophilic residues on another molecule. To preserve the specificity of binding, the polyethylene glycol groups can be blocked at one end (the end not bound to the protein) with a methyl ether group. Methods for pegylation of proteins and other polypeptide sequences are described in G. T. Hermanson, “Bioconjugate Techniques” (Academic Press, San Diego, 1996), pp. 605-618. Preferably, the intermediate release linker does not interact with the skin care agent or cosmeceutical and does not bind to or otherwise interact with the targeting moiety.

If a natural protein rich in thiol groups is desired, peptides derived from keratin can be used, and if long segments of hydrophobic residues are to be used for the endothermic attachment of nonpolar drugs, peptides derived from elastin, or biosynthesized sequences which mimic such sequences, can be used.

If the intermediate release linker is a non-protein polymer, it is typically polyethylene glycol, although analogous polymers, such as polypropylene glycol, can be used. When the intermediate release linker is polyethylene glycol, typically, the length of the polyethylene glycol chains, expressed in terms of the number of ethylene glycol monomers, is from about 10 ethylene glycol monomers to about 60 ethylene glycol monomers. Preferably, the length of the polyethylene glycol chains is from about 20 ethylene glycol monomers to about 40 ethylene glycol monomers. More preferably, the length of the polyethylene glycol chains is about 32 ethylene glycol monomers (i.e., polyethylene glycol with a molecular weight of about 2000 daltons).

Typically, the linkage between the skin care agent or cosmeceutical and the intermediate release linker is a covalent linkage, such as a covalent linkage involving the reactive moieties and the cross-linking agents described above, or other reactive moieties and cross-linking agents known in the art. In some alternatives, the therapeutic agent and the intermediate release linker can be derivatized by peptides, such as linkers such as TGEKP (SEQ ID NO: 85) and the longer linker TGGGGSGGGGTGEKP (SEQ ID NO: 86). Modifications of the longer linker of SEQ ID NO: 86 can also be used. For example, the polyglycine runs of four glycine (C) residues each can be of greater or lesser length (i.e., 3 or 5 glycine residues each). The serine residue (S) between the polyglycine runs can be replaced with threonine (T). The TGEKP (SEQ ID NO: 85) moiety that comprises part of the linker TGGGGSGGGGTGEKP (SEQ ID NO: 86) can be modified as described above for the TGEKP (SEQ ID NO: 85) linker alone. Still other linkers are known in the art and can alternatively be used. These include the linkers LRQKDGGGSERP (SEQ ID NO: 87), LRQKDGERP (SEQ ID NO: 88), GGRGRGRGRQ (SEQ ID NO: 89), QNKKGGSGDGKKKQHI (SEQ ID NO: 90), TGGERP (SEQ ID NO: 91), ATGEKP (SEQ ID NO: 92), and GGGSGGGGEGP (SEQ ID NO: 93), as well as derivatives of those linkers in which amino acid substitutions are made as described above for TGEKP (SEQ ID NO: 85) and TGGGGSGGGGTGEKP (SEQ ID NO: 86). For example, in these linkers, the serine (S) residue between the diglycine or polyglycine runs in QNKKGGSGDGKKKQHI (SEQ ID NO: 90) or GGGSGGGGEGP (SEQ ID NO: 93) can be replaced with threonine (T). In GGGSGGGGEGP (SEQ ID NO: 93), the glutamic acid (E) at position 9 can be replaced with aspartic acid (D). Other linkers such as glycine or serine repeats are well known in the art to link peptides such as single chain antibody domains. These linkers are described in United States Patent Application Publication No. 2007/0178499 by Barbas, Ill. Still other linkers are known in the art; some suitable linkers are described, for example in U.S. Pat. No. 6,936,439 to Mann et al. Such linkers typically comprise short oligopeptide regions that typically assume a random coil conformation. The linker typically consists of less than about 15 amino acid residues, more typically about 4 to 10 amino acid residues. When both the therapeutic drug and the intermediate release linker are derivatized by peptides, the linkage between the therapeutic agent and the intermediate release linker can then be a peptide (amide) bond formed between these peptides. In another alternative, the covalent linkage between the therapeutic agent and the intermediate release linker is a cleavable linker, such as, for example, cathepsin-cleavable linkers such as Val-Cit which are cleaved by intracellular cathepsins. Cleavable linkers include di-, tri-, and tetrapeptide subunits of cathepsin B, D, and L. Other cleavable linkers include acid-cleavable groups such as hydrazones which may be cleaved by endocytosis or through intracellular interaction with lysosomes. Still other cleavable linkers include acid-labile linkers. Examples of acid-labile linkers include linkers containing an orthoester group, a hydrazone, a cis-acetonyl, an acetal, a ketal, a silyl ether, a silazane, an imine, a citriconic anhydride, a maleic anhydride, a crown ether, an azacrown ether, a thiacrown ether, a dithiobenzyl group, a cis-aconitic acid, a cis-carboxylic alkatriene, methacrylic acid, and mixtures thereof. Examples of acid-labile groups and linkers are provided in U.S. Pat. No. 7,098,032 to Trubetskoy et al., U.S. Pat. No. 6,897,196 to Szoka, Jr., et al., U.S. Pat. No. 6,426,086 to Papahadjopolous et al., U.S. Pat. No. 7,138,382 to Wolff et al., U.S. Pat. No. 5,563,250 to Hylarides et al., and U.S. Pat. No. 5,505,931 to Pribish. Additional cleavable linkers include, but are not limited to, protease sensitive cleavable peptide linkers, nuclease sensitive cleavable nucleic acid linkers, lipase sensitive cleavable lipid linkers, glycosidase sensitive cleavable carbohydrate linkers, pH-sensitive cleavable linkers such as acid-labile cleavable linkers or base-labile cleavable linkers, photo-cleavable linkers, heat-labile cleavable linkers, cleavable linkers that are cleaved by the action of a hydrolytic enzyme (i.e., esterase cleavable linkers), and others. Cleavable linkers are described, for example, in United States Patent Application Publication No. 2010/0183727 by lannacone et al., United States Patent Application Publication No. 2010/0112042 by Polisky et al., United States Patent Application Publication No. 2010/0129392 by Shi et al., and United States Patent Application Publication No. 2010/0184831 by Hart et al. Cleavable linkers also include linkers containing disulfide groups, which can be cleaved by reduction, linkers containing glycols, which can be cleaved by periodate, linkers containing diazo groups, which can be cleaved by dithionite, linkers containing ester groups, which can be cleaved by hydroxylamine, and linkers containing sulfones, which can be cleaved by bases. Further details on such cleavable linkers are provided in G. T. Hermanson, “Bioconjugate Techniques” (Academic Press, San Diego, 1996), pp. 292-296.

In another alternative, the linkage between the intermediate release linker and the skin care agent or cosmeceutical can be a non-covalent linkage. If the linkage is a non-covalent linkage, it must be sufficiently stable to withstand storage and delivery and not be disrupted until the composition reaches its target cell, tissue, or organ, in particular, epidermal cells, in order to insure that targeting is specific. If the linkage between the intermediate release linker and the skin care agent or cosmeceutical is a non-covalent linkage, it is typically a biotin/avidin or biotin/streptavidin linkage. As used herein, the term “biotin” encompasses both biotin (hexahydro-2-oxo-1H-thieno[3,4-d]imidazole-4-pentanoic acid) itself, or its lysine derivative biocytin (c-N-biotinyl-L-lysine).

In yet another alternative, a specific antigen/antibody or hapten/antibody linkage can be used to couple the skin care agent or cosmeceutical and the intermediate release linker. As used herein, the term “antibody” includes all antibody derivatives with appropriate binding specificity, including naturally occurring antibodies, monoclonal antibodies, chimeric antibodies, humanized antibodies, and single-chain antibodies such as sFv antibody derivatives.

In a targeting composition according to the present invention, the intermediate release linker is further linked to the targeting moiety. Typically, the linkage between the intermediate release linker and the targeting moiety is a covalent linkage as described above; cleavable linkers can also be used in some alternatives, as described above. In some alternatives, either the intermediate release linker or the targeting moiety can be extended with a peptide linker such as described above. In some alternatives, the linkage between the intermediate release linker and the targeting moiety is a non-covalent linkage, such as a biotin/avidin or biotin/streptavidin. Other non-covalent linkages are possible alternatives, as described above, including antigen/antibody or hapten/antibody linkages.

In another alternative, the composition comprises a liposome having attached to its surface a peptide motif for binding the liposome to native collagen fibers.

When a liposome is employed, because of the nature of the relatively large nanoparticle we contemplate using (around 100 nm diameter, which represents around ⅓ the length of a collagen molecule, it may be desirable to insert multiple binding motifs on the surface of such a sphere to assure good linking and to stabilize its attachment. On the other hand it is possible that the protruding PEG chains may suffice to achieve this goal. In one alternative, the protruding peptides can be extended by inserting repeating sequences of glycine. Glycine provides maximum rotation around peptide bonds, and therefore maximal degree of motion. When such polyglycine extensions are employed, the polyglycine extensions typically range up to 50 repeating units. The displacement between the most adjacent binding sites among parallel oriented molecules is in the range of 2-4 nm. Free random movement of the glycine chains and the generation of as many attachments as possible are desirable. In another alternative, the extensions can be made more rigid, such as by using repeating Gly-Pro-Pro-Gly sequences) to generate a collagen like rigid triple helical extension radiating from surface of the liposome.

The properties of the liposomes that are employed in compositions in this alternative of the present invention depend on the chemotherapeutic agent to be carried by the liposomes, the details of the targeting process, pharmacokinetic considerations related to the persistence of the liposomes in the circulation and their clearance by the body, especially by the reticuloendothelial system.

Liposome charge also influences the electrostatic adsorption of proteins and, therefore, the clearance times and interactions with cells and tissues. For cationic complexes containing liposomes used in gene therapy, pronounced cytokine-related toxicity has been observed and is suggested to occur owing to enhanced accumulation and uptake of complexes including liposomes by Kupffer cells that would in turn activate inflammatory responses. The effect of negative charge of liposomes on complement activation has also been studied extensively. In general, it is preferred to use neutral or negatively-charged liposomes rather than positively-charged liposomes.

For long circulation times, pegylation of drug-carrier surfaces has been explored and has revolutionized in vivo drug delivery, including liposomes, micelles and proteins. Surface-grafted PEG chains, as a result of their high mobility and hydration in water, are thought to stabilize the liposome surface sterically and increase the circulation times of liposomes. In particular, pegylation results in long-circulating liposomes regardless of the liposome surface charge or the presence of cholesterol in the liposome membrane.

Accordingly, in compositions of the present invention, it is generally preferred to employ pegylated liposomes. However, the present invention is not limited to pegylated liposomes; other types of liposomes can be used, particularly if other means known in the art to increase liposome stability are employed.

It is suggested that PEG provides a steric barrier to protein adsorption on the liposome surface, that it alters liposome interactions with cells (including cells of the reticuloendothelial system [RES]) and/or that it results in reduced liposome aggregation. However, Applicants are not bound by this theory concerning the mechanism of increased stability liposomes that are derivatized with polyethylene glycol chains. The level of pegylation (PEG grafting density) does not seem to influence total protein adsorption from plasma but it does seem to influence the adsorption kinetics and the types or sizes of particular proteins that adsorb on the liposome surface. For short incubation times (2 minutes), low-grafted PEG densities in mushroom conformations (at concentrations not shielding the liposome surface entirely) almost abort specific binding between avidin and surface-grafted biotins and it was suggested that pegylation might have an effect on the diffusion of avidin on the liposome surface owing to the higher energy barrier presented by the grafted PEG chains, which, in a way, need to be “pushed” by avidin in order for the latter to bind to biotin. For the effect of pegylation in vivo, it has also been suggested that it might be a result partly of the role of PEG as a steric barrier to approaching macrophages by decreasing liposome uptake by these cells, which results in delayed clearance of PEG-modified liposomes, although adsorption of serum proteins might still occur on the liposome surface; in such cases, the adsorption of serum proteins on the liposome surface may not adversely impact the circulatory lifespan of such liposomes. Recently, in a well designed study, very strong evidence shows that a major mechanism by which pegylation extends circulation time would be to prohibit not the adsorption of proteins on the liposome surface but the approximation and aggregation of liposomes into larger liposomal structures. Aggregation, leading to the formation of larger structures (not necessarily resulting in fusion) of non-pegylated liposomes can explain their short circulation times.

Various approaches for surface pegylation have been pursued. Pegylated liposome surfaces with multiloop PEG chains containing hydrophobic “anchoring chains” protect liposomes from complement binding more than do grafted linear PEG chains and exhibit surface protection of the liposomes for longer times. Other pegylated surface architectures include branched PEG chains and tiered surface strategies with mixed lengths of extended PEG chains (potentially to present a molecular sieve for the different shapes and sizes of serum proteins); in vivo, these do seem to result in longer circulation times and higher tumor uptake than single-length grafted PEG chains. Any of these alternatives can be used in compositions according to the present invention. Commercial formulations (e.g., STEALTH® liposomes) contain grafted PEG at relatively high surface densities (5% mole for 2000 molecular weight [MW] PEG) that should result in extended brush conformations, with lengths that might extend beyond the surface of adsorbed proteins, providing the above-mentioned steric barrier. Pegylation, however, enhances accumulation of liposomes to the skin.

Other methods of derivatizing liposomes to enhance their circulatory lifespan or other desirable properties are known, including the use of monosialoganglioside (GM1) and phosphatidylinositol (PI), and are within the scope of the present invention. However, in most applications, the use of pegylated liposomes is preferred.

For small pegylated liposomes, the lipid composition probably determines the extent and types of surface-adsorbed plasma proteins that, in turn, affect specific liposome-cell interactions.

To relax the extracellular matrix (ECM) of the cells intended to take up the liposomes and enhance liposome penetration, different approaches have been followed. In one approach, hyaluronidase, the ECM-degrading enzyme, was administered.

Depending on the nature of delivered modalities including skin care agents or cosmeceuticals, cellular internalization of the targeting ligand and, therefore, of the targeted liposome, can be significant. However, cellular internalization is not required for all components of skin care agents or cosmeceuticals included in compositions according to the present invention.

Three different immobilization architectures of targeting ligands on liposomes exist. These immobilization architectures are: (1) type A liposomes, in which ligands are conjugated directly on the phospholipid headgroups of non-pegylated liposomes; (2) type B liposomes, in which ligands are conjugated directly on the phospholipid headgroups of pegylated liposomes; and (3) type C liposomes, in which ligands are conjugated on the free termini of pegylated chains. Type A liposomes bind to target cells specifically and exhibit fast blood clearance that is restored by the addition of grafted PEG chains and the formation of type B liposomes, which, however, exhibit reduced targeting owing to steric hindrance of the targeting ligand by the neighboring polymer chains. These observations lead to type C liposomes resulting in minimal screening of the ligand by the neighboring grafted PEG chains, however, in vivo, their circulation time was inversely proportional to their conjugated ligand-grafting density. In the case of type C liposomes in which the ligands are antibodies, removal of the antibody's Fc region can, in principle, restore longer circulation times.

Other alternatives employing compositions according to the present invention are also within the scope of the present invention. In one such alternative, two separate liposomal preparations with different targeting sites are administered to the same organism in need of treatment with a skin care agent or cosmeceutical. In another such alternative, known as pretargeting, a reagent that specifically binds to the liposomes and directly targets the epidermal region to be treated, such as a suitable monoclonal antibody, is administered first. In one alternative, the monoclonal antibody or other targeting reagent is conjugated to one of two binding partners that use the biotin-avidin link, while the liposome is conjugated to the other of the two binding partners that use the biotin-avidin link. In another alternative, the monoclonal antibody or other targeting reagent and the liposome are both conjugated to biotin or a derivative or analogue of biotin, and a biotin-binding component is introduced to cross-link the monoclonal antibody or other targeting reagent to the liposome. The avidin-binding component can be selected from the group consisting of avidin, streptavidin, a derivative or analogue of avidin or streptavidin, and a biotin-binding antibody. In still another approach, the liposomes can contain a derivatized component of a skin care agent or cosmeceutical that binds to a bispecific antibody, and, prior to the administration of the liposomes, the bispecific antibody is administered to the organism to be administered a skin care agent or cosmeceutical. The bispecific antibody binds both to the derivatized skin care agent or cosmeceutical included in the liposome and to a marker in epithelial cells of the organism. Alternatives for the epithelial cell marker, the skin care agent or cosmeceutical, and the bispecific antibody are known in the art and, for the skin care agent or cosmeceutical, are as described herein.

In still another approach, liposome compositions according to the present invention are used that include at least two antibodies, wherein each antibody is an antibody for a specific receptor on the surface of epithelial cells in the organism to be treated by administration of a skin care product or cosmeceutical, the receptors occurring in the same epithelial cell.

In yet another approach according to the present invention, a liposome composition according to the present invention has two functionalities in addition to the targeting moiety described above. These two functionalities are: (1) a binding functionality to a noninternalizing receptor on the surface of an epithelial cell to be targeted and (2) an initially hidden functionality through a cell-penetrating peptide that is activated only after binding to the cell surface at slightly acidic conditions.

One targeting moiety that can be included in liposomes to target them to epithelial cells, according to the present invention, is a peptide motif as described above that includes one or more CBDs as described above.

Accordingly, one aspect of the present invention is a composition comprising a liposome having attached to its surface a peptide motif (CBD) for binding the liposome to native collagen fibers.

Release of encapsulated contents is desired to occur after localization of liposomes after cellular uptake and accumulation into subcellular compartments to result in enhanced bioavailability of the skin care agent or cosmeceutical.

Accordingly, in one alternative of a composition according to the present invention, the diameter of the liposome is from about 50 nm to about 2000 nm. Typically, the diameter of the liposome is from about 200 nm to about 2000 nm. Preferably, the diameter of the liposome is about 1000 nm.

Accordingly, in another alternative of a composition according to the present invention, the liposome has polyethylene glycol (PEG) chains on its surface, i.e., is pegylated. Typically, the length of the polyethylene glycol chains, expressed in terms of the number of ethylene glycol monomers, is from about 10 ethylene glycol monomers to about 60 ethylene glycol monomers. Preferably, the length of the polyethylene glycol chains is from about 20 ethylene glycol monomers to about 40 ethylene glycol monomers. More preferably, the length of the polyethylene glycol chains is about 32 ethylene glycol monomers (i.e., polyethylene glycol with a molecular weight of about 2000 daltons).

When a composition according to the present invention has polyethylene glycol chains on the surface of the liposome, the polyethylene glycol chains can have either a free amino group or a free carboxyl group available for reaction. Alternatively, the polyethylene glycol chains can be derivatized with another reactive group that can be linked to a polypeptide or a therapeutic agent, such as a hydroxyl group or a carbonyl group.

In one preferred alternative, when a composition according to the present invention has polyethylene glycol chains on the surface of the liposome, and the polyethylene glycol chains have a free amino group, the peptide motif as described above can be linked to the polyethylene glycol chains through the formation of a peptide (amide) bond between the free amino group of the polyethylene glycol group and the free carboxyl group of the peptide motif. Alternatively, when the polyethylene glycol chains have free carboxyl groups, the peptide motif can be linked to the polyethylene glycol chains through the formation of a peptide (amide) bond between the free carboxyl group of the polyethylene glycol group and the free amino group of the peptide motif. These alternatives results in opposite orientations of the peptide motif.

The composition of the liposome is not critical; suitable proportions of ingredients for the preparation of liposomes are known in the art and are described, for example, in European Patent Application Publication No. EP 1332755 by Weng et al. In one alternative, the liposome can comprise from about 25% to about 35% of cholesterol, from about 60% to about 70% of dipalmitoylphosphatidylcholine (DPPC), and from about 2% to about 5% of a reactive pegylated lipid. The reactive pegylated lipid can be, but is not limited to, 1,2-distearoyl-sn-glycero-3-phosphoethanolamine-N-amino(polyethylene glycol-2000). Other reactive pegylated lipids can be used. In this alternative, preferably, the liposome comprises from about 27.5% to about 32.5% of cholesterol. In this alternative, more preferably, the liposome comprises about 30% of cholesterol.

The liposome can also include a small proportion (less than about 1%, typically less than 0.1%) of a standard fluorophore as a fluorescent marker to assist in determining the binding of the liposome to collagen. The fluorescent marker can be, but is not limited to, markers such as NDB II, which provides a green color, or rhodamine, which provides a pink color. The use of the fluorophore is optional and its omission does not interfere with the activity of the liposome. The fluorophore can be used for identifying and localizing liposomes clinically and in experimental studies to determine efficacy of the procedure.

Liposome compositions according to the present invention can be prepared according to standard liposome preparation techniques known in the art, such as those described in European Patent Application Publication No. EP 1332755 by Weng et al., supra. The peptide motif as described above can be either attached after liposome synthesis or attached to one of the ingredients of the liposome prior to liposome assembly. Typically, the peptide motif is attached to the reactive pegylated lipid as described above, although the peptide motif can alternatively be attached to one of the other components of the liposome. Typically, as described above, the peptide motif is attached through a free carboxyl group on the peptide motif to an amino group on the reactive PEG moiety, forming a peptide (amide) bond. In another alternative, the peptide motif is attached through a free amino group on the peptide motif to a carboxyl group on the reactive PEG moiety, again, forming a peptide bond. In other alternatives, other cross-linking methods as known in the art can be used for cross-linking the peptide motif to the liposome. These cross-linking methods are discussed below.

Additionally, a composition according to the present invention comprising a liposome having attached to its surface a peptide motif for binding the liposome to native collagen fibers, as described above, can further comprise a skin care agent or cosmeceutical as described above. The skin care agent or cosmeceutical can be either incorporated in the interior of the liposome or attached to the surface of the liposome.

Therefore, when liposomes are used, another aspect of the present invention is a composition comprising a liposome having attached to its surface a peptide motif for binding the liposome to native collagen fibers. The peptide motif is one of the collagen binding domains described above. The composition further comprises a skin care agent or cosmeceutical; alternatives for skin care agents and cosmeceuticals are also described above. The skin care agent or cosmeceutical can be incorporated in the interior of the liposome; alternatively, the skin care agent or cosmeceutical can be attached to the surface of the liposome. The liposome can further comprise a substance that can be identified by a radiological procedure selected from the group consisting of X-ray, MRI, and CT. The substance can be selected from the group consisting of a radio-opaque substance and a radioactive substance. When liposomes are employed, a pharmaceutical composition can comprise:

(1) a therapeutically effective quantity of the composition of the present invention with liposomes, including a skin care agent or cosmeceutical as described above; and

(2) a pharmaceutically acceptable carrier, diluent, or excipient in unit dosage form.

Another aspect of the present invention is a pharmaceutical composition comprising:

(1) a therapeutically effective quantity of at least one targeting composition according to the present invention as described above including therein a skin care agent or cosmeceutical as described above; and

(2) a pharmaceutically acceptable carrier, diluent, or excipient.

Skin care agents and cosmeceuticals that can be included in compositions according to the present invention, whether such compositions employ liposomes or other targeting mechanisms, are as described above.

In another alternative, the pharmaceutical composition can include a first targeting composition that includes a skin care agent and a second targeting composition that includes a cosmeceutical. Other combinations of two or more targeting compositions in a pharmaceutical composition according to the present invention are also possible. For example, and not by way of limitation, the pharmaceutical composition includes two different skin care agents. In yet another alternative, the pharmaceutical composition includes two different cosmeceuticals. In yet another alternative, a pharmaceutical composition according to the present invention can include: (i) at least one targeting composition according to the present invention as described above; and (ii) at least one additional skin care agent or cosmeceutical. Various combinations are again possible. For example, and not by way of limitation, the pharmaceutical composition can include: (i) a targeting composition including a first skin care agent; and (ii) a second skin care agent. As another alternative, the pharmaceutical composition can include: (i) a targeting composition including a skin care agent; and (ii) a cosmeceutical. As still another alternative, the pharmaceutical composition can include: (i) a targeting composition including a cosmeceutical; and (ii) a skin care agent. As yet another alternative, the pharmaceutical composition can include: (i) a targeting composition including a first cosmeceutical and (ii) a second cosmeceutical. Still other combinations are possible.

In yet another alternative, bone morphogenetic proteins (BMPs) or active portions thereof can be targeted to such areas of exposed collagen for cosmeceutical purposes, such as, for example, to assist in epidermal smoothing or regularization, removal or reduction of wrinkles, or to act synergistically with conventional dermal fillers. Bone morphogenetic proteins usable as cosmeceuticals are described further below.

The BMPs are described in further detail in the following publications: (1) F. P. Luyten et al., “Purification and Partial Amino Acid Sequence of Osteogenin, a Protein Initiating Bone Differentiation,” J. Biol. Chem. 264: 13377-13380 (1989); (2) E. Özkaynak et al., “Murine Osteogenic Protein (OP-1): High Levels of mRNA in Kidney,” Biochem. Biophys. Res. Commun. 179: 116-123 (1991); (3) R. M. Harland et al., “The Transforming Growth Factor β Family and Induction of the Vertebrate Mesoderm: Bone Morphogenetic Proteins are Ventral Inducers,” Proc. Natl. Acad. Sci. USA 91: 10243-10246 (1994); (4) S. K. Maiti & G. R. Singh, “Bone Morphogenetic Proteins-Novel Regulators of Bone Formation,” Ind. J. Exp. Biol. 36: 237-244 (1998); (5) J. M. Wozney et al., “Novel Regulators of Bone Formation: Molecular Clones and Activities,” Science 242: 1528-1534 (1988); (6) D. M. Kingsley et al., “What Do BMPs Do in Mammals? Clues from the Mouse Short-Ear Mutation,” Trends Genet. 10: 16-21 (1994); (7) C. Scheufler et al., “Crystal Structure of Human Bone Morphogenetic Protein-2 at 2.7 Å Resolution,” J. Mol. Biol. 287: 103-115 (1999); (8) J. Q. Feng et al., “Structure and Sequence of Mouse Bone Morphogenetic Protein-2 Gene (BMP-2): Comparison of the Structures and Promoter Regions of BMP-2 and BMP-4 Genes,” Biochim. Biophys. Acta 1218: 221-224 (1994); (9) N. Ghosh-Choudhury et al., “Expression of the BMP 2 Gene During Bone Cell Differentiation,” Crit. Rev. Eukaryot. Gene Expr. 4: 345-355 (1994); (10) B. L. Rosenzweig et al., “Cloning and Characterization of a Human Type II Receptor for Bone Morphogenetic Proteins,” Proc. Natl. Acad. Sci. USA 92: 7632-7636; (11) L. J. Jonk et al., “Identification and Functional Characterization of a Smad Binding Element (SBE) in the JunB Promoter That Acts as a Transforming Growth Factor-βActivin, and Bone-Morphogenetic-Protein-Inducible Enhancer,” J. Biol. Chem. 273: 21145-21152 (1998); and (12) M. Kawabata et al., “Signal Transduction by Bone Morphogenetic Proteins,” Cytokine Growth Factor Rev. 9: 49-61 (1998). The BMPs represent a family of proteins that initiate, promote, and maintain cartilage and bone morphogenesis, differentiation and regeneration in both the developing embryo and the adult. There are more than 30 known BMPs, of which 15 are found in mammals. BMPs belong to the transforming growth factor β (TGFβ) superfamily, which includes TGFβs, activins/inhibins, Mullerian-inhibiting substance (MIS) and glial cell line-derived neurotrophic factor. Comparison and alignment of the amino acid sequences of BMPs reveal that BMPs, except for BMP-1, share a common structural motif that is distinct from the structure of BMP-1. These BMPs include BMP-2, BMP-3, BMP-3b, BMP4, BMP-5, BMP-6, BMP-7, BMP-8, BMP-8B, GDF-3, GDF-5, GDF-6, GDF-7, GDF-8, GDF-9, GDF-10, and nodal. In this specification, the term “BMP,” without further qualification, is to be taken to include BMP-1; the term “BMP sharing a common structural motif” is to be taken to include BMPs other than BMP-1. These BMPs sharing a common structural motif are disulfide-linked dimeric proteins. BMP-1 is not properly a BMP family member; rather it is a procollagen C proteinase related to Drosophila tolloid and which is postulated to regulate BMP activity through proteolysis of BMP antagonists/binding proteins. These growth factors can exist in multiple forms, such as: (1) splicing variants produced from mRNAs generated by spicing from alternative sites; (2) variants produced by proteolysis, such as the cleavage of signal peptides or propeptides; (3) variants produced by the presence or lack of glycosylation, typically N-linked glycosylation; (4) naturally-occurring isoforms; (5) naturally-occurring mutations or allelic variants; and (6) artificial variants produced by genetic engineering in which one or more amino acids in the primary sequence are altered by techniques such as site-specific mutagenesis; such artificial variants are frequently designated muteins. In general, these multiple forms are within the scope of the present invention when they exist or can be produced for a particular growth factor. Additionally, growth factors useful in compositions according to the present invention can be incorporated into fusion proteins. Examples of fusion proteins incorporating bone morphogenetic proteins are disclosed in U.S. Pat. No. 6,352,972 to Nimni et al. In general, such fusion proteins are also within the scope of the present invention when they exist or can be generated. These fusion proteins can incorporate multiple domains or domains from more than one naturally-occurring growth factor. They can also incorporate elements such as reporter genes or detection tags. Therefore, the use of such fusion proteins incorporating growth factors, including but not limited to BMPs, is within the scope of the invention. The BMPs can be BMPs sharing a common structural motif and that are disulfide-linked dimeric proteins, such as, but not limited to, BMP-2, BMP-3, BMP-3b, BMP-4, BMP-5, BMP-6, BMP-7, BMP-8, BMP-8B, GDF-3, GDF-5, GDF-6, GDF-7, GDF-8, GDF-9, GDF-10, and nodal. A particularly preferred BMP for incorporation into a fusion protein is BMP-3.

In yet another alternative, a composition according to the present invention can include a growth factor. Growth factors also can be targeted to such areas of exposed collagen for cosmeceutical purposes, such as, for example, to assist in epidermal smoothing or regularization, removal or reduction of wrinkles, or to act synergistically with conventional dermal fillers. Growth factors suitable for incorporation into compositions according to the present invention include, but are not limited to, adrenomedullin (AM), autocrine mobility factor, bone morphogenetic proteins (BMPs) (considered to be growth factors and covered above), epidermal growth factor (EGF), erythropoietin (EPO), fibroblast growth factor (FGF), granulocyte-colony stimulating factor (G-CSF), granulocyte-macrophage colony stimulating factor (GM-CSF), growth differentiation factor-9 (GDF-9), hepatocyte growth factor (HGF), hepatoma derived growth factor (HDGF), insulin-like growth factor (IGF), migration stimulating factor (MSF), myostatin (GDF-8), nerve growth factor (NGF), brain-derived neurotrophic factor (BDNF), novel neurotrophin-1 (NNT-1), neurotrophin-3 (NT-3), neurotrophin-4 (NT-4), platelet-derived growth factor (PDGF), thrombopoietin (TPO), transforming growth factor α (TGF-α), transforming growth factor β (TGF-β), vascular endothelial growth factor (VEGF), and placental growth factor (PGF); other growth factors are known in the art. A particularly suitable growth factor is GM-CSF.

In yet another alternative of a therapeutic composition according to the present invention, the therapeutic composition comprises: (1) a skin care agent or cosmeceutical as described above; (2) an intermediate release linker bound to the therapeutic agent; (3) a targeting moiety bound to the intermediate release linker; and (4) a growth factor bound either to the polypeptide or protein skin care agent or cosmeceutical or the intermediate release linker. The growth factor that is bound either to the polypeptide or protein skin care agent or cosmeceutical or to the intermediate release linker is covalently linked to the polypeptide or protein skin care agent or cosmeceutical or to the intermediate release linker by one of the coupling reactions described above, depending on the functional groups available on the growth factor and the polypeptide for crosslinking as described above. In some alternatives, the active agent of the skin care agent or cosmeceutical is a protein or polypeptide.

Still another aspect of the invention is a method of treating a subject with a skin care agent or cosmeceutical as described above to effect an esthetic improvement in the subject comprising administration of an effective quantity of a targeting composition to the subject. The esthetic improvement can be, but is not limited to, removal or reduction of blemishes, removal or reduction of wrinkles, removal or reduction of irregularities in skin color or skin tone, or other esthetic improvements. This method comprises administration of a therapeutically effective quantity of a targeting composition according to the present invention as described above. The targeting composition can include the optional carrier component.

The targeting compositions comprising a skin care agent or cosmeceutical as described above can be administered directly to subjects in need of treatment. In an alternative, targeting compositions according to the present invention comprising a skin care agent or cosmeceutical are preferably administered to the subjects in pharmaceutical compositions which comprise the targeting composition comprising the skin care agent or cosmeceutical, and, optionally, at least one additional skin care agent or cosmeceutical in a therapeutically effective dose. Typically, the pharmaceutical composition includes a pharmaceutically acceptable carrier. When at least one additional skin care agent or cosmeceutical is present in the pharmaceutical composition, and the pharmaceutical composition includes a pharmaceutically acceptable carrier, the pharmaceutically acceptable carrier is compatible with any additional skin care agent or cosmeceutical present in the pharmaceutical composition. Pharmaceutically acceptable carriers are agents which are not biologically or otherwise undesirable, i.e., the agents can be administered to a subject along with the targeting composition comprising the skin care agent or cosmeceutical without causing any undesirable biological or esthetic effects or interacting in a deleterious manner with any of the components of the pharmaceutical composition in which it is contained. Pharmaceutically acceptable carriers enhance or stabilize the composition, or can facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically acceptable carrier should be suitable for various routes of administration described herein. Typically, the pharmaceutically acceptable carrier is a dermatologically acceptable carrier as described below. Suitable additional skin care agents or cosmeceuticals for inclusion in a pharmaceutical composition are as described above.

A pharmaceutical composition containing a therapeutic composition incorporating a skin care agent or cosmeceutical and, optionally, other skin care agents or cosmeceuticals can be administered by a variety of methods known in the art. Typically, administration is performed topically, by direct application of the composition to the skin to be treated. However, in some alternatives, administration may be performed by other routes, such as subcutaneous injection or systemic administration such as oral administration.

Pharmaceutical compositions according to the present invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds.

Actual dosage levels of the active ingredients in the pharmaceutical compositions of the present invention can be varied so as to obtain an amount of the active ingredient which is effective to achieve the desired esthetic response for a particular subject, composition, and mode of administration, without being toxic to the subject. The selected dosage level depends upon a variety of pharmacokinetic factors including the activity of the particular skin care agent or cosmeceutical, the route of administration, the time of administration, the rate of excretion of the particular compound being employed, the severity of the condition, other health considerations affecting the subject, and the status of liver and kidney function of the subject. It also depends on the duration of the treatment and other factors known in the art, such as, but not limited to, other drugs, skin care agents, cosmeceuticals, compounds and/or materials used in combination with the particular skin care agent or cosmeceutical employed, as well as the age, weight, condition, general health and prior medical history of the subject being treated, and like factors. Methods for determining optimal dosages are described in the art, e.g., Remington: The Science and Practice of Pharmacy, Mack Publishing Co., 20th ed., 2000. Typically, an esthetically effective dosage would be between about 0.001 and 100 mg/kg body weight of the subject to be treated. Similar considerations apply if additional skin care agents or cosmeceutical are administered as described above.

The targeting composition or pharmaceutical composition that includes the targeting composition, and, if desired, other skin care agents or cosmeceuticals as described above, are usually administered to the subjects on multiple occasions. Intervals between single dosages can be weekly, monthly or yearly. Intervals can also be irregular as indicated by esthetic response or other parameters well known in the art. Alternatively, the targeting composition or pharmaceutical composition can be administered as a sustained release formulation, in which case less frequent administration is required. Dosage and frequency vary depending on the half-life in the subject of the skin care agent or cosmeceutical and the other components included in a pharmaceutical composition.

In some alternatives, oral administration of compositions according to the present invention may be employed. Preparations formulated for oral administration may be in the form of tablets, dragees, capsules, or solutions. The pharmaceutical compositions contemplated by the present invention may be manufactured in a manner that is itself known, e.g., by means of conventional mixing, dissolving, granulating, dragee-making, levitating, emulsifying, encapsulating, entrapping or lyophilizing processes. Pharmaceutical preparations for oral use can be obtained by combining the compositions with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different doses of therapeutic agent. Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-a-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

Although not generally preferred, parenteral administration may be utilized in some circumstances. Pharmaceutical formulations for parenteral administration can include aqueous solutions or suspensions. Suitable lipophilic solvents or vehicles include fatty oils such as sesame oil or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility or dispersibility of the composition to allow for the preparation of highly concentrated solutions.

Sustained-release formulations or controlled-release formulations are well-known in the art. For example, the sustained-release or controlled-release formulation can be (1) an oral matrix sustained-release or controlled-release formulation; (2) an oral multilayered sustained-release or controlled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by this reference.

One of ordinary skill in the art can readily prepare formulations for controlled release or sustained release comprising a targeting composition according to the present invention by modifying the formulations described above, such as according to principles disclosed in V. H. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the targeting composition, such as aqueous solubility, partition coefficient, molecular size, stability, and nonspecific binding to proteins and other biological macromolecules. This process of preparation also takes into account biological factors, such as absorption, distribution, metabolism, duration of action, the possible existence of side effects, and margin of safety, for the targeting composition. Accordingly, one of ordinary skill in the art could modify the formulations into a formulation having the desirable properties described above for a particular application.

Compositions according to the present invention typically comprise a dermatologically acceptable carrier. The phrase “dermatologically acceptable carrier,” as used herein, means that the carrier is suitable for topical application to the keratinous tissue, has good aesthetic properties, is compatible with the skin care agents or cosmeceuticals of the present invention and any other components, and will not cause any untoward safety or toxicity concerns. A safe and effective amount of carrier is from about 50% to about 99.99%, preferably from about 80% to about 99.9%, more preferably from about 90% to about 98%, and even more preferably from about 90% to about 95% of the composition.

A number of alternatives for components of dermatologically acceptable carriers and combinations of such components for use in compositions according to the present invention are described below.

The carrier can be in a wide variety of forms. For example, emulsion carriers, including, but not limited to, oil-in-water, water-in-oil, water-in-oil-in-water, and oil-in-water-in-silicone emulsions, are useful herein. Preferred carriers contain an emulsion such as oil-in-water emulsions, water-in-oil emulsions, and water-in-silicone emulsions. Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.01% to about 10%, more preferably from about 0.1% to about 5%, of an emulsifier, based on the weight of the carrier. Emulsifiers may be nonionic, anionic or cationic. Suitable emulsifiers are disclosed in, for example, U.S. Pat. No. 3,755,560 to Dickert et al., and in U.S. Pat. No. 4,421,769 to Dixon et al. The emulsion may also contain an anti-foaming agent to minimize foaming upon application to the keratinous tissue. Anti-foaming agents include high molecular weight silicones and other materials well known in the art for such use. Suitable emulsions may have a wide range of viscosities, depending on the desired product form. Exemplary low viscosity emulsions, which are preferred, have a viscosity of about 50 centistokes or less, more preferably about 10 centistokes or less, still more preferably about 5 centistokes or less. Preferred water-in-silicone and oil-in-water emulsions are described in greater detail below.

(I) Water-in-Silicone Emulsions

Water-in-silicone emulsions contain a continuous silicone phase and a dispersed aqueous phase.

(A) Continuous Silicone Phase

Preferred water-in-silicone emulsions of the present invention contain from about 1% to about 60%, preferably from about 5% to about 40%, more preferably from about 10% to about 20%, by weight of a continuous silicone phase. The continuous silicone phase exists as an external phase that contains or surrounds the discontinuous aqueous phase described hereinafter. The continuous silicone phase contains a polyorganosiloxane oil. The continuous silicone phase of these preferred emulsions contain between about 50% and about 99.9% by weight of organopolysiloxane oil and less than about 50% by weight of a non-silicone oil. In an especially preferred embodiment, the continuous silicone phase contains at least about 50%, preferably from about 60% to about 99.9%, more preferably from about 80% to about 99.9%, polyorganosiloxane oil by weight of the continuous silicone phase, and up to about 50% non-silicone oils, preferably less than about 30%, even more preferably less than about 10%, and even more preferably less than about 2%, by weight of the continuous silicone phase. Water-in-silicone emulsions of this type are described in PCT Application Publication No. WO 97/21423 by Robinson et al.

The organopolysiloxane oil for use in the composition may be volatile, non-volatile, or a mixture of volatile and non-volatile silicones. The term “nonvolatile” as used in this context refers to those silicones that are liquid under ambient conditions and have a flash point (under one atmosphere of pressure) of or greater than about 100° C. The term “volatile” as used in this context refers to all other silicone oils. Examples of suitable organopolysiloxane oils include polyalkylsiloxanes, cyclic polyalkylsiloxanes, and polyalkylarylsiloxanes. Polyalkylsiloxanes useful in the composition herein include polyalkylsiloxanes with viscosities of from about 0.5 to about 1,000,000 centistokes at 25° C. Such polyalkylsiloxanes can be represented by the general chemical formula R₃SiO[R₂SiO]ₓSiR₃ wherein R is an alkyl group having from one to about 30 carbon atoms (preferably R is methyl or ethyl, more preferably methyl; also mixed alkyl groups can be used in the same molecule), and x is an integer from 0 to about 10,000, chosen to achieve the desired molecular weight which can range to over about 10,000,000. Commercially available polyalkylsiloxanes include the polydimethylsiloxanes, which are also known as dimethicones. Suitable dimethicones include those represented by the chemical formula (CH₃)₃SiO[(CH₃)2SiO]ₓ[CH₃RSiO]_{y}Si(CH₃)₃ wherein R is straight or branched chain alkyl having from two to about 30 carbon atoms and x and y are each integers of 1 or greater selected to achieve the desired molecular weight which can range to over about 10,000,000. Examples of these alkyl-substituted dimethicones include cetyl dimethicone and lauryl dimethicone. Cyclic polyalkylsiloxanes suitable for use in the composition include those represented by the chemical formula [SiR2-O]ₙ wherein R is an alkyl group (preferably R is methyl or ethyl, more preferably methyl) and n is an integer from about 3 to about 8, more preferably n is an integer from about 3 to about 7, and still more preferably n is an integer from about 4 to about 6. When R is methyl, these materials are typically referred to as cyclomethicones. Also useful are materials such as trimethylsiloxysilicate, which is a polymeric material corresponding to the general chemical formula [(CH₂)₃SiO_{1/2}]ₓ[SiO₂]y, wherein x is an integer from about 1 to about 500 and y is an integer from about 1 to about 500. Dimethiconols are also suitable for use in the composition. These compounds can be represented by the chemical formulas R₃SiO[R₂SiO]ₓSiR₂OH and HOR₂SiO[R₂SiO]ₓSiR₂OH wherein R is an alkyl group (preferably R is methyl or ethyl, more preferably methyl) and x is an integer from 0 to about 500, chosen to achieve the desired molecular weight. Commercially available dimethiconols are typically sold as mixtures with dimethicone or cyclomethicone. Polyalkylaryl siloxanes are also suitable for use in the composition. Polymethylphenyl siloxanes having viscosities from about 15 to about 65 centistokes at 25° C. are especially useful. As stated above, the continuous silicone phase may contain one or more non-silicone oils. Suitable non-silicone oils have a melting point of about 25° C. or less under about one atmosphere of pressure.

(B) Dispersed Aqueous Phase

The topical compositions of the present invention contain from about 30% to about 90%, more preferably from about 50% to about 85%, and still more preferably from about 70% to about 80% of a dispersed aqueous phase. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The aqueous phase can be water, or a combination of water and one or more water soluble or dispersible ingredients. Examples of such ingredients include, but are not limited to, thickeners, acids, bases, salts, chelating agents, gums, water-soluble or dispersible alcohols and polyols, buffers, preservatives, sunscreening agents, colorings, and the like. The topical compositions of the present invention will typically contain from about 25% to about 90%, preferably from about 40% to about 80%, more preferably from about 60% to about 80%, of water in the dispersed aqueous phase by weight of the composition.

(C) Emulsifier for Dispersing the Aqueous Phase

The water-in-silicone emulsions of the present invention preferably contain an emulsifier. In a preferred embodiment, the composition contains from about 0.1% to about 10% emulsifier, more preferably from about 0.5% to about 7.5%, still more preferably from about 1% to about 5%, emulsifier by weight of the composition. The emulsifier helps disperse and suspend the aqueous phase within the continuous silicone phase. A wide variety of emulsifying agents can be employed herein to form the preferred water-in-silicone emulsion, provided that the selected emulsifying agent is chemically and physically compatible with components of the composition of the present invention, and provides the desired dispersion characteristics. Suitable emulsifiers include silicone emulsifiers, non-silicone-containing emulsifiers, and mixtures thereof, known by those skilled in the art for use in topical personal care products. Preferably these emulsifiers have an HLB value of or less than about 14, more preferably from about 2 to about 14, and still more preferably from about 4 to about 14. Emulsifiers having an HLB value outside of these ranges can be used in combination with other emulsifiers to achieve an effective weighted average HLB for the combination that falls within these ranges. Silicone emulsifiers are preferred. A wide variety of silicone emulsifiers are useful herein. These silicone emulsifiers are typically organically modified organopolysiloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C₂-C₃₀ pendant side chains. Still other useful dimethicone copolyols include materials having various cationic, anionic, amphoteric, and zwitterionic pendant moieties. The dimethicone copolyol emulsifiers useful herein can be described by the following general structure (Formula (DCE-I):

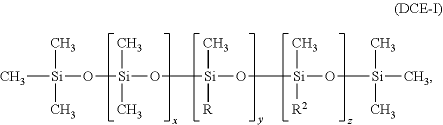

wherein:

(1) R is C₁-C₃₀ straight, branched, or cyclic alkyl; and

(2) R² is selected from the group consisting of —(CH₂)ₙ—O—(CH₂CHR³O)ₘ—H and —(CH₂)ₙ—O—(CH₂CHR³O)ₘ—(CH₂CHR⁴O)ₒ—H;

wherein n is an integer from 3 to about 10; R³ and R⁴ are selected from the group consisting of H and C₁-C₆ straight or branched chain alkyl such that R³ and R⁴ are not simultaneously the same; and m, o, x, and y are selected such that the molecule has an overall molecular weight from about 200 to about 10,000,000, with m, o, x, and y being independently selected from integers of zero or greater such that m and o are not both simultaneously zero, and z being independently selected from integers of 1 or greater. Also useful herein, although not strictly classified as dimethicone copolyols, are silicone surfactants as depicted in the structures in the previous paragraph wherein R² is —(CH₂)n-O—R₅, wherein R⁵ is a cationic, anionic, amphoteric, or zwitterionic moiety. Examples of dimethicone copolyols and other silicone surfactants useful as emulsifiers herein include polydimethylsiloxane polyether copolymers with pendant polyethylene oxide side chains, polydimethylsiloxane polyether copolymers with pendant polypropylene oxide side chains, polydimethylsiloxane polyether copolymers with pendant mixed polyethylene oxide and polypropylene oxide side chains, polydimethylsiloxane polyether copolymers with pendant mixed poly(ethylene)(propylene)oxide side chains, polydimethylsiloxane polyether copolymers with pendant organobetaine side chains, polydimethylsiloxane polyether copolymers with pendant carboxylate side chains, polydimethylsiloxane polyether copolymers with pendant quaternary ammonium side chains; and also further modifications of the preceding copolymers containing pendant C₂-C₃₀ straight, branched, or cyclic alkyl moieties. Suitable dimethicone copolyols include cetyl dimethicone copolyol, lauryl dimethicone copolyol, dimethicone copolyol acetate, diemethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, and dimethicone copolyol stearate. Dimethicone copolyol emulsifiers are also described in U.S. Pat. No. 4,960,764 to Figueroa, Jr. et al. Among the non-silicone-containing emulsifiers useful herein are various non-ionic and anionic emulsifying agents such as sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C₁-C₃₀ fatty alcohols, polyglyceryl esters of C₁-C₃₀ fatty acids, C₁-C₃₀ esters of polyols, C₁-C₃₀ ethers of polyols, alkyl phosphates, polyoxyalkylene fatty ether phosphates, fatty acid amides, acyl lactylates, soaps, and mixtures thereof. Other non-silicone-containing emulsifiers can be used, including polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

(II) Oil-in-Water Emulsions

Other preferred topical carriers include oil-in-water emulsions, having a continuous aqueous phase and a hydrophobic, water-insoluble phase (“oil phase”) dispersed therein. Examples of suitable oil-in-water emulsion carriers are described in U.S. Pat. No. 5,073,371 to Turner et al. and U.S. Pat. No. 5,073,372 to Turner et al. An especially preferred oil-in-water emulsion, containing a structuring agent, hydrophilic surfactant and water, is described in detail herein.

(A) Structuring Agent

A preferred oil-in-water emulsion contains a structuring agent to assist in the formation of a liquid crystalline gel network structure. Without being limited by theory, it is believed that the structuring agent assists in providing rheological characteristics to the composition which contribute to the stability of the composition. The structuring agent may also function as an emulsifier or surfactant. Preferred compositions of this invention contain from about 0.1% to about 20%, more preferably from about 1% to about 10%, even more preferably from about 1% to about 5%, by weight of the composition, of a structuring agent. The preferred structuring agents of the present invention include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof. More preferred structuring agents of the present invention are selected from stearyl alcohol, cetyl alcohol, behenyl alcohol, the polyethylene glycol ether of stearyl alcohol having an average of about 2 ethylene oxide units (steareth-2), the polyethylene glycol ether of stearyl alcohol having an average of about 21 ethylene oxide units (steareth-21), the polyethylene glycol ether of cetyl alcohol having an average of about 2 ethylene oxide units, and mixtures thereof.

(B) Hydrophilic Surfactant

The preferred oil-in-water emulsions contain from about 0.05% to about 10%, preferably from about 1% to about 6%, and more preferably from about 1% to about 3% of at least one hydrophilic surfactant which can disperse the hydrophobic materials in the water phase (percentages by weight of the topical carrier). The surfactant, at a minimum, must be hydrophilic enough to disperse in water. Preferred hydrophilic surfactants are selected from nonionic surfactants. Among the nonionic surfactants that are useful herein are those that can be broadly defined as condensation products of long chain alcohols, e.g. C₈-C₃₀ alcohols, with sugar or starch polymers, i.e., glycosides. These compounds can be represented by the formula (S)ₙ—O—R wherein S is a sugar moiety such as glucose, fructose, mannose, and galactose; n is an integer of from about 1 to about 1000, and R is a C₈-C₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C₈-C₂₀ alkyl group, and n is an integer of from about 1 to about 9; suitable examples include decyl polyglucoside and lauryl polyglucoside. Other useful nonionic surfactants include the condensation products of alkylene oxides with fatty acids (i.e. alkylene oxide esters of fatty acids). These materials have the general formula RCO(X)ₙOH wherein R is a C₁₀-C₃₀ alkyl group, X is —OCH₂CH₂— (i.e. derived from propylene glycol or oxide), and n is an integer from about 6 to about 200. Other nonionic surfactants are the condensation products of alkylene oxides with 2 moles of fatty acids (i.e. alkylene oxide diesters of fatty acids). These materials have the general formula RCO(X)ₙOOCR wherein R is a C₁₀-C₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 100. Other nonionic surfactants are the condensation products of alkylene oxides with fatty alcohols (i.e. alkylene oxide ethers of fatty alcohols). These materials have the general formula R(X)ₙOR′ wherein R is a C₁₀-C₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 100 and R′ is H or a C10-30 alkyl group. Still other nonionic surfactants are the condensation products of alkylene oxides with both fatty acids and fatty alcohols, i.e. wherein the polyalkylene oxide portion is esterified on one end with a fatty acid and etherified (i.e. connected via an ether linkage) on the other end with a fatty alcohol. These materials have the general formula RCO(X)ₙOR′ wherein R and R′ are C₁₀-C₃₀ alkyl groups, X is —OCH₂CH₂— (i.e. derived from ethylene glycol or oxide) or —OCH₂CHCH₃— (i.e. derived from propylene glycol or oxide), and n is an integer from about 6 to about 100. Examples of these alkylene oxide derived nonionic surfactants include ceteth-6, ceteth-10, ceteth-12, ceteareth-6, ceteareth-10, ceteareth-12, steareth-6, steareth-10, steareth-12, steareth-21, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof. Still other useful nonionic surfactants include polyhydroxy fatty acid amide surfactants corresponding to the structural formula (PFA-I):

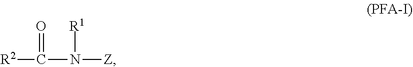

wherein R¹ is H, C₁-C₄ alkyl, 2-hydroxyethyl, 2-hydroxypropyl, preferably C₁-C₄ alkyl, more preferably methyl or ethyl, most preferably methyl; R² is C₅-C₃₁ alkyl or alkenyl, preferably C₇-C₁₉ alkyl or alkenyl, more preferably C₉-C₁₇ alkyl or alkenyl, most preferably C₁₁-C₁₅ alkyl or alkenyl; and Z is a polyhydroxyhydrocarbyl moiety having a linear hydrocarbyl chain with at least 3 hydroxyls directly connected to the chain, or an alkoxylated derivative (preferably ethoxylated or propoxylated) thereof. Z preferably is a sugar moiety selected from the group consisting of glucose, fructose, maltose, lactose, galactose, mannose, xylose, and mixtures thereof. An especially preferred surfactant corresponding to the above structure is coconut alkyl N-methyl glucoside amide (i.e., wherein the R²CO— moiety is derived from coconut oil fatty acids. Preferred among the nonionic surfactants are those selected from the group consisting of steareth-21, ceteareth-20, ceteareth-12, sucrose cocoate, steareth-100, PEG-100 stearate, and mixtures thereof. Other nonionic surfactants suitable for use herein include sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C₁-C₃₀ fatty alcohols, polyglyceryl esters of C₁-C₃₀ fatty acids, C₁-C₃₀ esters of polyols, C₁-C₃₀ ethers of polyols, alkyl phosphates, polyoxyalkylene fatty ether phosphates, fatty acid amides, acyl lactylates, and mixtures thereof. Examples of these emulsifiers include: polyethylene glycol 20 sorbitan monolaurate (Polysorbate 20), polyethylene glycol 5 soya sterol, steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof. Another group of nonionic surfactants useful herein include the fatty acid ester blends based on a mixture of sorbitan or sorbitol fatty acid ester and sucrose fatty acid ester, the fatty acid in each instance being preferably C₈-C₂₄, more preferably C₁₀-C₂₀. The preferred fatty acid ester emulsifier is a blend of sorbitan or sorbitol C₁₆-C₂₀ fatty acid ester with sucrose C₁₀-C₁₆ fatty acid ester, especially sorbitan stearate and sucrose cocoate. Other suitable surfactants useful herein include a wide variety of cationic, anionic, zwitterionic, and amphoteric surfactants such as are known in the art, including surfactants described in U.S. Pat. No. 5,011,681 to Ciotti et al.; U.S. Pat. No. 4,421,769 to Dixon et al.; and U.S. Pat. No. 3,755,560 to Dickert et al. The hydrophilic surfactants useful herein can contain a single surfactant, or any combination of suitable surfactants. The exact surfactant (or surfactants) chosen will depend upon the pH of the composition and the other components present. The cationic surfactants useful herein include dialkyl quaternary ammonium compounds, examples of which are described in U.S. Pat. Nos. 5,151,209; 5,151,210; 5,120,532; 4,387,090; 3,155,591; 3,929,678; and 3,959,461. The cationic surfactants useful herein also include cationic ammonium salts such as those having the formula (CA-I):

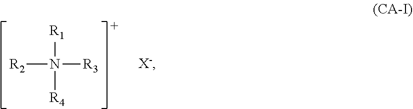

wherein R₁ is an alkyl group having from about 12 to about 30 carbon atoms, or an aromatic, aryl or alkaryl group having from about 12 to about 30 carbon atoms; R₂, R₃, and R₄ are independently selected from hydrogen, an alkyl group having from about 1 to about 22 carbon atoms, or aromatic, aryl or alkaryl groups having from about 12 to about 22 carbon atoms; and X is any compatible anion, preferably selected from chloride, bromide, iodide, acetate, phosphate, nitrate, sulfate, methyl sulfate, ethyl sulfate, tosylate, lactate, citrate, glycolate, and mixtures thereof. Additionally, the alkyl groups of R₁, R₂, R₃, and R₄ can also contain ester and/or ether linkages, or hydroxy or amino group substituents (e.g., the alkyl groups can contain polyethylene glycol and polypropylene glycol moieties). More preferably, R₁ is an alkyl group having from about 12 to about 22 carbon atoms; R₂ is selected from H or an alkyl group having from about 1 to about 22 carbon atoms; R₃ and R₄ are independently selected from H or an alkyl group having from about 1 to about 3 carbon atoms; and X is as described above. Alternatively, other useful cationic emulsifiers include amino-amides, wherein in the above structure R₁ is alternatively R₅CONH—(CH₂)ₙ, wherein R₅ is an alkyl group having from about 12 to about 22 carbon atoms, and n is an integer from about 2 to about 6, more preferably from about 2 to about 4. Examples of these cationic emulsifiers include stearamidopropyl PG-dimonium chloride phosphate, behenamidopropyl PG-dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof. Especially preferred is behenamidopropyl PG dimonium chloride. Examples of quaternary ammonium salt cationic surfactants include those selected from cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, and mixtures thereof. Additional quaternary ammonium salts include those wherein the C₁₂ to C₃₀ alkyl carbon chain is derived from a tallow fatty acid or from a coconut fatty acid. The term “tallow” refers to an alkyl group derived from tallow fatty acids (usually hydrogenated tallow fatty acids), which generally have mixtures of alkyl chains in the C₁₆ to C₁₅ range. The term “coconut” refers to an alkyl group derived from a coconut fatty acid, which generally have mixtures of alkyl chains in the C₁₂ to C₁₄ range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof. An example of a quaternary ammonium compound having an alkyl group with an ester linkage is ditallowyl oxyethyl dimethyl ammonium chloride. More preferred cationic surfactants are those selected from behenamidopropyl PG dimonium chloride, dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, stearamidopropyl PG-dimonium chloride phosphate, stearamidopropyl ethyldiammonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof. A preferred combination of cationic surfactant and structuring agent is behenamidopropyl PG dimonium chloride and/or behenyl alcohol, wherein the ratio is preferably optimized to enhance physical and chemical stability, especially when such a combination contains ionic and/or highly polar solvents. This combination is especially useful for delivery of sunscreening agents such as zinc oxide and octyl methoxycinnamate. A wide variety of anionic surfactants are also useful herein. Suitable anionic surfactants are disclosed in U.S. Pat. No. 3,929,678 to Laughlin et al. Examples of anionic surfactants include the alkoyl isethionates, and the alkyl and alkyl ether sulfates. Examples of alkoyl isethionates include ammonium cocoyl isethionate, sodium cocoyl isethionate, sodium lauroyl isethionate, sodium stearoyl isethionate, and mixtures thereof. Another suitable class of anionic surfactants are the water-soluble salts of the organic sulfuric acid products of the general formula R₁—SO₃-M, wherein R₁ is a straight or branched chain, saturated aliphatic hydrocarbon radical having from about 8 to about 24, preferably about 10 to about 16, carbon atoms; and M is a cation. Still other anionic synthetic surfactants include the class designated as succinamates, olefin sulfonates having about 12 to about 24 carbon atoms, and β-alkyloxy alkane sulfonates. Examples of these materials are sodium lauryl sulfate and ammonium lauryl sulfate. Other anionic materials useful herein are soaps (i.e. alkali metal salts, e.g., sodium or potassium salts) of fatty acids, typically having from about 8 to about 24 carbon atoms, preferably from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, or other alternatives known in the art). The fatty acids can also be synthetically prepared. Soaps are described in more detail in U.S. Pat. No. 4,557,853 to Collins. Amphoteric and zwitterionic surfactants are also useful herein. Examples of amphoteric and zwitterionic surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 22 carbon atoms (preferably C₈-C₁₈) and one contains an anionic water solubilizing group, e.g., carboxy, sulfonate, sulfate, phosphate, or phosphonate. Examples are alkyl iminoacetates, and iminodialkanoates and aminoalkanoates of the formulas RN[(CH₂)ₘCO₂M]₂ and RNH(CH₂)ₘCO₂M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Examples of suitable amphoteric surfactants include sodium 3-dodecyl-aminopropionate, sodium 3-dodecylaminopropane sulfonate, N-alkyltaurines such as the one prepared by reacting dodecylamine with sodium isethionate according to the teaching of U.S. Pat. No. 2,658,072 to Kosmin; N-higher alkyl aspartic acids such as those produced according to U.S. Pat. No. 2,438,091 to Lynch; and metal salts of substituted quaternary hydroxy cycloimidinic acid metal alcoholates described in U.S. Pat. No. 2,528,378 to Mannheimer, which is incorporated herein by reference in its entirety. Other examples of useful amphoterics include phosphates, such as coamidopropyl PG-dimonium chloride phosphate. Other amphoteric or zwitterionic surfactants useful herein include betaines. Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as Lonzaine 16SP from Lonza Corp.), lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and amidobetaines and amidosulfobetaines (wherein the RCONH(CH₂)₃ radical is attached to the nitrogen atom of the betaine), oleyl betaine, and cocamidopropyl betaine. Other useful amphoteric and zwitterionic surfactants include the sultaines and hydroxysultaines such as cocamidopropyl hydroxysultaine and the alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and trialkanolamine (e.g., triethanolamine), a preferred example of which is sodium lauroyl sarcosinate.

(C) Water

In this alternative, the compositions may contain from about 25% to about 98%, preferably from about 65% to about 95%, more preferably from about 70% to about 90% water by weight of the topical carrier. The hydrophobic phase is dispersed in the continuous aqueous phase. The hydrophobic phase may contain water insoluble or partially soluble materials such as are known in the art, including but not limited to the silicones described herein in reference to silicone-in-water emulsions, and other oils and lipids such as described above in reference to emulsions.

Additionally, compositions according to the present invention, including but not limited to lotions and creams, may contain a dermatologically acceptable emollient. Such compositions preferably contain from about 1% to about 50% of the emollient. As used herein, “emollient” refers to a material useful for the prevention or relief of dryness, as well as for the protection of the skin. A wide variety of suitable emollients is known and may be used herein; in some alternatives, a preferred emollient is glycerol. Glycerol is preferably used in an amount of from or about 0.001% to or about 30%, more preferably from or about 0.01% to or about 20%, still more preferably from or about 0.1% to or about 10%, e.g., 5%. Lotions and creams according to the present invention generally contain a solution carrier system and one or more emollients. Lotions and creams typically contain from about 1% to about 50%, preferably from about 1% to about 20%, of emollient; and from about 50% to about 90%, preferably from about 60% to about 80%, water. Creams are generally thicker than lotions due to higher levels of emollients and/or higher levels of thickeners.

In some alternatives, compositions according to the present invention can be in the form of an ointment. Ointments of the present invention may contain a simple carrier base of animal or vegetable oils or semi-solid hydrocarbons (oleaginous); absorption ointment bases which absorb water to form emulsions; or water soluble carriers, e.g., a water soluble solution carrier. Ointments may further contain a thickening agent; suitable thickening agents can include, but are not limited to, beeswax, cocoa butter, shea butter, wool wax, and cetyl alcohol. Other thickening agents include, but are not limited to: (i) carboxylic acid polymers; (ii) crosslinked polyacrylate polymers; (iii) polyacrylamide polymers; (iv) polysaccharides; and (v) gums. Carboxylic acid polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Suitable carboxylic acid polymers are described in U.S. Pat. No. 5,087,445 to Haffey et al.; U.S. Pat. No. 4,509,949 to Huang et al.; and U.S. Pat. No. 2,798,053, to Brown. Suitable carboxylic acid polymers include, but are not limited to: carbomers, which are homopolymers of acrylic acid crosslinked with allyl ethers of sucrose or pentaerythritol; copolymers of C₁₀-C₃₀ alkyl acrylates with one or more monomers of acrylic acid, methacrylic acid, or one of their short-chain (i.e., C₁₄) alcohol esters, wherein the crosslinking agent is an allyl ether of sucrose or pentaerythritol. Crosslinked polyacrylate polymers include both cationic and nonionic polymers. Crosslinked polyacrylate polymers are described in U.S. Pat. No. 5,100,660 to Hawe et al.; U.S. Pat. No. 4,849,484 to Heard; U.S. Pat. No. 4,835,206 to Farrar et al.; U.S. Pat. No. 4,628,078 to Glover et al.; and U.S. Pat. No. 4,599,379 to Flesher et al. Polyacrylamide polymers include nonionic polyacrylamide polymers, including substituted branched or unbranched polymers. Suitable polyacrylamide polymers include a polymer produced by cross-linking polymerized 2-acrylamido-2-methylpropanesulfonic acid with N,N′-methylenebisacrylamide; one formulation also includes C₁₃-C₁₄ isoparaffin and laureth-7. Other polyacrylamide polymers useful herein include multi-block copolymers of acrylamides and substituted acrylamides with acrylic acids and substituted acrylic acids. Suitable polysaccharide polymers include, but are not limited to, cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful are the alkyl-substituted celluloses. In these polymers, the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is cetyl hydroxyethylcellulose. Other useful polysaccharides include scleroglucans which are a linear chain of (1→3) linked glucose units with a (1→6) linked glucose every three units. Gums are primarily derived from natural sources but may also be inorganic. Suitable gums include, but are not limited to, acacia, agar, algin, alginic acid, ammonium alginate, amylopectin, calcium alginate, calcium carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, and xanthan gum.

Ointments may further contain an emollient. For example, an ointment may contain from about 2% to about 10% of an emollient; and from about 0.1% to about 2% of a thickening agent.

In some alternatives, compositions according to the present invention can include at least one ingredient useful for cleansing of the skin. In such an alternative, the compositions typically contain from about 1% to about 90%, preferably from about 5% to about 10%, of a dermatologically acceptable surfactant. The surfactant is suitably selected from anionic, nonionic, zwitterionic, amphoteric and ampholytic surfactants, as well as mixtures of these surfactants. Examples of possible surfactants include isoceteth-20, sodium methyl cocoyl taurate, sodium methyl oleoyl taurate, and sodium lauryl sulfate. Suitable surfactants are described in U.S. Pat. No. 4,800,197 to Kowcz et al.

In some alternatives, additional components can be included in a composition according to the present invention. These additional components generally fall under the category of dermatologically acceptable carriers, diluents, or excipients. These additional components include, but are not limited to, abrasives, absorbents, fragrances, pigments, colorants, essential oils, skin sensates, astringents (e.g., clove oil, menthol, camphor, eucalyptus oil, eugenol, menthyl lactate, witch hazel distillate), anti-caking agents, anti-foaming agents, antioxidants, binders, biological additives, buffering agents, bulking agents, film formers (e.g., a copolymer of eicosene and vinyl pyrrolidone), opacifying agents, pH adjusters, propellants, sequestrants, acidifying agents, alkalinizing agents, complexing agents, and penetration enhancers. Suitable additional components are disclosed in PCT Patent Application Publication No. WO 2002/076423 by Bissett et al.

Suitable buffering agents include, but are not limited to, acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate, sodium bicarbonate, Tris (Tris(hydroxymethyl)aminomethane), MOPS (3-(N-morpholino)propanesulfonic acid), HEPES (N-(2-hydroxyethyl)piperazine-N′-(2-ethanesulfonic acid), ACES (2-[(2-amino-2-oxoethyl)amino]ethanesulfonic acid), ADA (N-(2-acetamido)2-iminodiacetic acid), AMPSO (3-[(1,1-dimethyl-2-hydroxyethylamino]-2-propanesulfonic acid), BES (N,N-bis(2-hydroxyethyl)-2-aminoethanesulfonic acid, Bicine (N,N-bis(2-hydroxyethylglycine), Bis-Tris (bis-(2-hydroxyethyl)imino-tris(hydroxymethyl)methane, CAPS (3-(cyclohexylamino)-1-propanesulfonic acid), CAPSO (3-(cyclohexylamino)-2-hydroxy-1-propanesulfonic acid), CHES (2-(N-cyclohexylamino)ethanesulfonic acid), DIPSO (3-[N,N-bis(2-hydroxyethylamino]-2-hydroxy-propanesulfonic acid), HEPPS (N-(2-hydroxyethylpiperazine)-N′-(3-propanesulfonic acid), HEPPSO (N-(2-hydroxyethyl)piperazine-N′-(2-hydroxypropanesulfonic acid), MES (2-(N-morpholino)ethanesulfonic acid), triethanolamine, imidazole, glycine, ethanolamine, phosphate, MOPSO (3-(N-morpholino)-2-hydroxypropanesulfonic acid), PIPES (piperazine-N,N′-bis(2-ethanesulfonic acid), POPSO (piperazine-N, N′-bis(2-hydroxypropaneulfonic acid), TAPS (N-tris[hydroxymethyl)methyl-3-aminopropanesulfonic acid), TAPSO (3-[N-tris(hydroxymethyl)methylamino]-2-hydroxy-propanesulfonic acid), TES (N-tris(hydroxymethyl)methyl-2-aminoethanesulfonic acid), tricine (N-tris(hydroxymethyl)methylglycine), 2-amino-2-methyl-1,3-propanediol, and 2-amino-2-methyl-1-propanol.

Suitable acidifying agents include, but are not limited to, acetic acid, citric acid, fumaric acid, hydrochloric acid, diluted hydrochloric acid, malic acid, nitric acid, phosphoric acid, diluted phosphoric acid, sulfuric acid, and tartaric acid.

Suitable antioxidants include, but are not limited to, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite, sodium thiosulfate, sulfur dioxide, and tocopherol.

Suitable alkalinizing agents include, but are not limited to, strong ammonia solution, ammonium carbonate, diethanolamine, diisopropanolamine, potassium hydroxide, sodium bicarbonate, sodium borate, sodium carbonate, sodium hydroxide, and trolamine.

Suitable complexing agents include, but are not limited to, ethylenediaminetetraacetic acid, salts of ethylenediaminetetraacetic acid, gentisic acid ethanolamide, and oxyquinoline sulfate.

Suitable penetration enhancers include, but are not limited to, monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones, and ureas.

Another aspect of the present invention is a method of treating a subject with a skin care agent or cosmeceutical as described above to effect an esthetic improvement in the subject. The method comprises administering a therapeutically effective quantity of a targeting composition according to the present invention to effect an esthetic improvement in the subject. The esthetic improvement can be, but is not limited to, selected from the group consisting of removal or reduction of blemishes, removal or reduction of wrinkles, and removal or reduction of irregularities in skin color or skin tone. Typically, the targeting composition is administered topically. Typically, the targeting composition includes the optional carrier component. In one alternative, the targeting composition is administered in a pharmaceutical composition including at least one additional skin care agent or cosmeceutical.

The invention is illustrated by the following Examples. These Examples are included for illustrative purposes only, and are not intended to limit the invention. These Examples may describe prospective work to the extent that is described in the future tense as appropriate.

### Example 1

### Targeting of Peptides to Collagen

Studies with targeted peptides used the decapeptide sequence: Trp-Arg-Glu-Pro-Ser-Phe-Met-Ala-Leu-Ser (WREPSFMALS) (SEQ ID NO: 1). It was linked to either TGF-β or BMP-3 at the C-terminus of the corresponding growth factors. These covalently linked growth factors retained significant biological activities and developed a strong binding affinity towards native collagen.

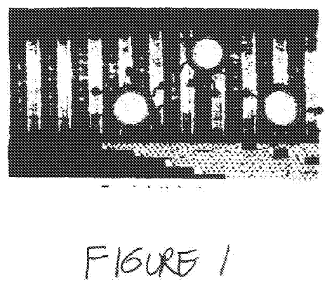

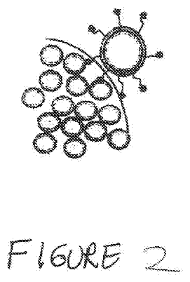

Because of the nature of the relatively large nanoparticle we contemplate using for the targeting composition (around 100 nm diameter, which represents around ⅓ the length of a collagen molecule (see FIG. 1), we believe that we may have to insert multiple binding motifs on the surface of such a sphere to assure good linking and to stabilize its attachment. On the other hand it is possible that protruding PEG chains may suffice to achieve this goal. If we have to do so we will extend the protruding peptides by inserting repeating sequences of glycine (also shown in FIG. 1). Glycine provides maximum rotation around peptide bonds because of the small size of the side chain (a hydrogen atom) and minimum steric hindrance, and therefore maximal degree of motion. We have selected polyglycine extensions ranging from zero and 50 repeating units. The displacement between the most adjacent binding sites among parallel oriented molecules is in the range of 2-4 nm. We want to allow for free random movement of the glycine chains, and of course generate as many attachments as possible. Initially we will experiment with the lower molecular weight enhanced extensions. In one alternative, the extension can be made more rigid (i.e., by using repeating Gly-Pro-Pro-Gly sequences) to generate a collagen-like rigid triple helical extension radiating from the targeting conjugate.

FIG. 1 shows a native collagen fiber stained with phosphotungstic acid, showing 68-nm periodicity and a schematic representation of collagen molecules measuring approximately 300 nm (adapted from M. Nimni, ed., “Collagen”, Vol. 1, CRC Press, 1988).

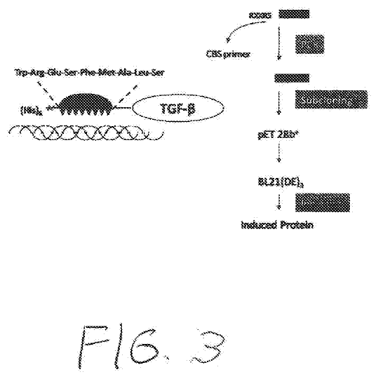

FIG. 2 shows the molecular packing of the Type I collagen fiber.

Polypeptide growth factors, of the TGF-β family, and others, were constructed to contain, at the C-terminal end, a decapeptide with high affinity for collagen (Andrades, Nimni et al. 1996; Tuan, Cheung et al. 1996; Andrades, Han et al. 1999; Hall, Han et al. 2001; Han, Perelman et al. 2002; Romijn, Westein et al. 2003). This was intended to provide concentration of the growth factors where needed for maximum efficacy. In compositions and methods according to the present invention, this effect can be used for delivery of the skin care agent or cosmeceutical to the skin.

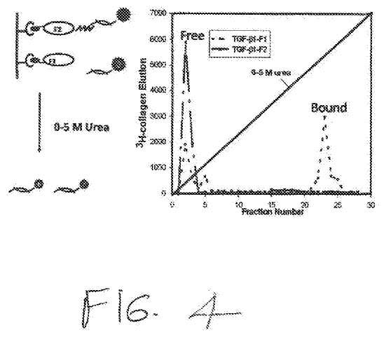

FIG. 3 depicts a genetically engineered fusion protein consisting of TGF-β1 with a collagen binding decapeptide. The purification tag comprises a hexapeptide of histidine, linked via a Gly-Gly link; it binds tightly to a Ni-NTA column for purification. Although the growth factor TGF-β1, in general, is not characterized as a skin care agent or cosmeceutical, it can be used in this context to demonstrate collagen binding and the capacity to be delivered effectively to the tissue or organ of interest; in the case of compositions and methods according to the present invention, the tissue or organ of interest is the skin or a defined region of the skin for delivery of a skin care agent or cosmeceutical.

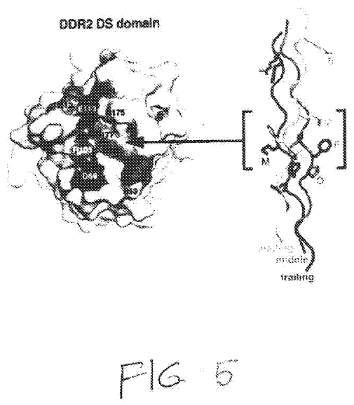

FIG. 4 depicts the binding of the TGF-β with a collagen binding domain to collagen; the binding requires a high concentration of urea for dissociation. This is compared to the behavior of TGF-β without the collagen binding domain, which has poor affinity for collagen.

Suitable CBDs to be used are as described above, including, but not limited to, the original von Willebrand derived polypeptide binding sequence, namely WREPSFCALS (SEQ ID NO: 1) or a slight variant. We will compare this with the fibronectin binding sequence Gly-Gly-Trp-Ser-His-Trp (GGWSHW) (SEQ ID NO: 94) derived from thrombospondin, as well as variants of the CBP with insertions, permutations, and modifications and, if practical, combinations separated by suitable spacers. In particular, the decapeptide of SEQ ID NO: 1 involves a series of exposed amino acids, located strategically within the N-terminus, in an area extending from residues 570 (F) to 682 of Von Willebrand factor (Takagi, Asai et al. 1992). By binding competition this decapeptide was found to bind, on a molar basis, 20 times more efficiently to collagen than the intact VWF (Takagi, Asai et al. 1992). Further examination of the crystal structure of the collagen binding regions of VWF A-3 Domain (Ichikawa, Osawa et al. 2007); (Romijn, Westein et al. 2003); (Staelens, Hadders et al. 2006) as well as the complementary collagen exposed surface (Lisman, Raynal et al. 2006) is expected to yield CBDs with increased binding affinity.

Collagens are large, triple-helical proteins that form fibrils and network-like structures in the extracellular matrix. They have played a major role in the evolution of metazoans from their earliest origins. Cell adhesion receptors that interact with collagen such as the integrins are at least as old as the collagens (Heino, Huhtala et al. 2009); (Whittaker and Hynes 2002) and instrumental in the evolution of bone, cartilage, and the immune system in chordates. In vertebrates collagen binding receptor tyrosine kinases send signals into cells after adhesion to collagen. Nevertheless, collagen continues to be seen primarily as an inert scaffold. To us the value of using it as a target became most relevant when we observed that it is only at sites of rapid tissue remodeling that collagen fibers become devoid of their normal proteoglycan coating, and therefore recognizable as such. This provides a basis for the use of compositions according to the present invention for the delivery of skin care agents or cosmeceuticals, as it is at such sites where the need for the delivery of such skin care agents or cosmeceuticals is most important.

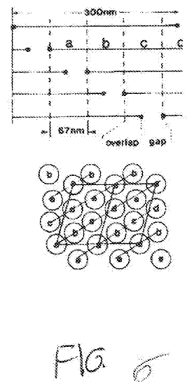

Other CBD, such as the discoidin domain receptors, DDR1 and DDR2, are receptor tyrosine kinases known to be activated by native triple-helical collagen. The sequence on collagen that binds DDR2 with highest affinity has similarity to the binding site for von Willebrand's factor, GVMGFO (O is hydroxyproline). (Konitsiotis, Raynal et al. 2008). The scattered amino acids on the binding site on the ligand are highlighted (FIG. 5). (Brondijk, de Ruiter et al.). The complete amino acid sequence of wild-type human DDR2 is shown in FIG. 5. A peptide discovered in the process of mapping the topography of collagen is P-15, a synthetic 15 residue peptide which binds to collagen at the single mammalian collagenase cleavage city (Gough and Bhatnagar 1999). The P-15 peptide, characterized as GTPGPGGIAGQRGVV (SEQ ID NO: 19) has found clinical application in the area of bone mineralization. The single unique collagenase cleavage site may be particularly interesting since it becomes exposed during periods of active collagen remodeling, and thus may represent a site for delivery of skin care agents or cosmeceuticals by compositions according to the present invention.

FIG. 5 shows molecular modeling of discoidin, including the amino acids on the surface involved in binding to collagen. These amino acids and their distribution within the DS domain provide a three-dimensional view of the nature of the collagen-ligand interaction.

As part of our intention to enhance binding affinities we will increase the number of CBD's, properly spaced from each other (FIG. 7). Peptide (B) will be designed to match the profile of the skin care agent or cosmeceutical it carries and amino acid sequences inserted and crosslinking mechanisms will adjusted to the hydrophobic or electrostatic character of such a skin care agent or cosmeceutical. The basic motifs will be selected from the group consisting of: Trp-Arg-Glu-Pro-Ser-Phe-Met-Ala-Leu-Ser (WREPSFMALS) (SEQ ID NO: 1); Trp-Arg-Glu-Pro-Ser-Phe-Cys-Ala-Leu-Ser (WREPSFCALS) (SEQ ID NO: 2) and peptides related by one or more conservative amino acid substitutions. Alternatively we will expand to a group consisting of: GPPGWREPSFMALSGPPG) (SEQ ID NO: 9), GPPGWREPSFCALSGPPG (SEQ ID NO: 10), and GPPGWRDPSFMALSGPPG (SEQ ID NO: 11), thus adding a “collagen like” sequence at one or both ends. Such sequences have been previously generated as well as CNBr peptides by cleavage of the native collagen molecule (Deshmukh and Nimni 1973). Such peptides fold and generate small size stable triple helical structures (“mini-collagens”), thermodynamically favored at 37° C., which should enhance binding to the fibers.

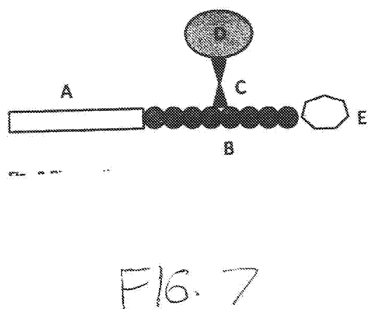

FIG. 6 is a schematic drawing of molecular packing within a collagen fiber. (A) Axial view showing linear staggering; (B) Cross-sectional view showing the unit cell. (B) shows how particular segments are repeated on the surface of the fiber (b-b for instance is separated by 2× the diameter of a molecule or approximately 3 nm laterally, the distance that repeating CBDs should be set apart for optimal binding).

Conservative amino substitution will also be explored. These can include (original residue followed by possible substitution): Ala/Gly or Ser; Arg/Lys; Asp/Glu; Cys/Ser; Gln/Asn; Gly/Asp; Gly/Ala or Pro. (Creighton (1984) Proteins, W. H. Freeman and Company; Schuiz and Schimer (1979) Principles of Protein Structure, Springer-Verlag). Certain conservative substitutions, positive or negatively charged, may improve binding affinity. Ideally we would like to include in our CBDs amino acids such as those highlighted in FIG. 5.

FIG. 7 is a diagrammatic representation of a collagen targeting vector: (A) CBD; (B) peptide for facilitating skin care agent or cosmeceutical attachment (D) attachment (length of peptide and specific amino acids in peptide leading to suitable conformations in solution will vary); (C) reactive functional groups suitable for skin care agent or cosmeceutical attachment (—SH, —CO₂H, —NH₂, or other groups); (D) skin care agent or; (E) additional site for identical or different CBD, separated by a suitable length of spacer (B) can be added.

FIG. 8 shows the entire wild-type DDR2 amino acid sequence.

If a peptide is selected from an internal sequence of a protein, terminal amidation (C-terminus) or acetylation (N-terminus) will remove its charge. In addition, this modification makes the resulting peptide more stable towards enzymatic degradation by exopeptidases. Biotin and fluorescein isothiocyanate (FITC) are activated precursors used for fluorescein labeling. For efficient N-terminal labeling, a seven-atom aminohexanoyl spacer (NH₂—CH₂—CH₂—CH₂—CH₂—CH₂—COOH) will be inserted between the fluorophore (fluorescein) and the N-terminus of the peptide. One common means of conjugation involves the use of maleimide, which couples N or C terminus cysteine residues of the peptide to the carrier protein.

In vitro binding studies will initially be carried out as described in a number of publications. In addition, in order to generate a more biocompatible and representative model, we will generate surfaces of native collagen, collagen/PG composites, reconstituted collagen fibers coated with supernatants of tissue homogenates, and other collagen-containing constructs to resemble the “masked” collagen present in tissues. In the past we plated reconstituted fibrous collagen (monomeric collagen assembled into fibers by heating to 37° C.) in petri dishes for the purpose of evaluating agents that inhibited crosslinking and or/enzymatic degradation (Nimni 1968). Selected areas of a reconstituted collagen fibrous network coated with proteoglycans will be enclosed by removable cylindrical inserts. Some will be treated with MMP's to expose “naked” collagen molecules on the fiber surface, to simulate what happens at sites of active collagen remodeling; such sites are particularly desirable for targeting of a skin care agent or cosmeceutical to the site. Others will remain masked by the surface deposited non-collagen extracellular matrix glycoproteins. The selective affinity of the various CBD's towards the exposed collagen will be evaluated using specific histochemical stains, as well as built in fluorescent or other markers. The sequences to be evaluated will be derived from sequences derived from conformational analysis, and will include the simplest CBD now in use and variables with collagen compatible peptide conformations, spacers to bridge repeating motifs on the surface of collagen, separated by distances estimated from the pattern of molecular assembly, coiling conformation, and other molecular parameters. Binding constants will be quantified, and the constructs with highest binding affinities as drug carriers will be selected. Evaluation of binding constants will be aided by coupling fluorescent markers to the peptides.

### REFERENCES

The following references are cited, and are hereby incorporated herein by this reference. The inclusion of these references is not to be taken as an admission that they are prior art.
Andrades, J. A., B. Han, et al. (1999). “A recombinant human TGF-beta1 fusion protein with collagen-binding domain promotes migration, growth, and differentiation of bone marrow mesenchymal cells.” Exp Cell Res 250(2): 485-98.
Andrades, J. A., M. E. Nimni, et al. (1996). “Type I collagen combined with a recombinant TGF-beta serves as a scaffold for mesenchymal stem cells.” Int J Dev Biol Suppl 1: 107S-108S.
Brondijk, T. H., T. de Ruiter, et al. (2010). “Crystal structure and collagen-binding site of immune inhibitory receptor LAIR-1: unexpected implications for collagen binding by platelet receptor GPVI.” Blood 115(7): 1364-73.
Chawla, S. P., V. S. Chua, et al. (2010). “Advanced phase I/II studies of targeted gene delivery in vivo: intravenous Rexin-G for gemcitabine-resistant metastatic pancreatic cancer.” Mol Ther 18(2): 435-41.
Chawla, S. P., V. S. Chua, et al. (2009). “Phase I/II and phase II studies of targeted gene delivery in vivo: intravenous Rexin-G for chemotherapy-resistant sarcoma and osteosarcoma.” Mol Ther 17(9): 1651-7.
Deshmukh, K. and M. E. Nimni (1973). “Isolation and characterization of cyanogen bromide peptides from the collagen of bovine articular cartilage.” Biochem J 133(4): 615-22.
Farndale, R. W., T. Lisman, et al. (2008). “Cell-collagen interactions: the use of peptide Toolkits to investigate collagen-receptor interactions.” Biochem Soc Trans 36(Pt 2): 241-50.
Gordon, E. M. and F. L. Hall (2009). “The ‘timely’ development of Rexin-G: first targeted injectable gene vector (review).” Int J Oncol 35(2): 229-38.
Gough, C. A. and R. S. Bhatnagar (1999). “Differential stability of the triple helix of (Pro-Pro-Gly)10 in H2O and D2O: thermodynamic and structural explanations.” J Biomol Struct Dyn 17(3): 481-91.
Hall, F. L., B. Han, et al. (2001). “Phenotypic differentiation of TGF-beta1-responsive pluripotent premesenchymal prehematopoietic progenitor (P4 stem) cells from murine bone marrow.” J Hematother Stem Cell Res 10(2): 261-71.
Han, B. (1998). “Collagen targeting TGF-beta: expression, characterization, and applications.” Ph. D. Thesis.
Han, B., N. Perelman, et al. (2002). “Collagen-targeted BMP3 fusion proteins arrayed on collagen matrices or porous ceramics impregnated with Type I collagen enhance osteogenesis in a rat cranial defect model.” J Orthop Res 20(4): 747-55.
Heino, J., M. Huhtala, et al. (2009). “Evolution of collagen-based adhesion systems.” Int J Biochem Cell Biol 41(2): 341-8.
Herr, A. B. and R. W. Farndale (2009). “Structural insights into the interactions between platelet receptors and fibrillar collagen.” J Biol Chem 284(30): 19781-5.
Ichikawa, O., M. Osawa, et al. (2007). “Structural basis of the collagen-binding mode of discoidin domain receptor 2.” EMBO J 26(18): 4168-76.
Karmali, P. P., V. R. Kotamraju, et al. (2009). “Targeting of albumin-embedded paclitaxel nanoparticles to tumors.” Nanomedicine 5(1): 73-82.
Konitsiotis, A. D., N. Raynal, et al. (2008). “Characterization of high affinity binding motifs for the discoidin domain receptor DDR2 in collagen.” J Biol Chem 283(11): 6861-8.
Lisman, T., N. Raynal, et al. (2006). “A single high-affinity binding site for von Willebrand factor in collagen III, identified using synthetic triple-helical peptides.” Blood 108(12): 3753-6.
Nimni, M. E. (1968). “A defect in the intramolecular and intermolecular cross-linking of collagen caused by penicillamine. I. Metabolic and functional abnormalities in soft tissues.” J Biol Chem 243(7): 1457-66.
Nimni, M. E. (1997). “Polypeptide growth factors: targeted delivery systems.” Biomaterials 18(18): 1201-25.
Romijn, R. A., E. Westein, et al. (2003). “Mapping the collagen-binding site in the von Willebrand factor-A3 domain.” J Biol Chem 278(17): 15035-9.
Ruoslahti, E., S. N. Bhatia, et al. (2010). “Targeting of drugs and nanoparticles to tumors.” J Cell Biol 188(6): 759-68.
Sharkey, R. M. and D. M. Goldenberg (2005). “Perspectives on cancer therapy with radiolabeled monoclonal antibodies.” J Nucl Med 46 Suppl 1: 115S-27S.
Staelens, S., M. A. Hadders, et al. (2006). “Paratope determination of the antithrombotic antibody 82D6A3 based on the crystal structure of its complex with the von Willebrand factor A3-domain.” J Biol Chem 281(4): 2225-31.
Takagi, J., H. Asai, et al. (1992). “A collagen/gelatin-binding decapeptide derived from bovine propolypeptide of von Willebrand factor.” Biochemistry 31(36): 8530-4.
Tuan, T. L., D. T. Cheung, et al. (1996). “Engineering, expression and renaturation of targeted TGF-beta fusion proteins.” Connect Tissue Res 34(1): 1-9.
Whittaker, C. A. and R. O. Hynes (2002). “Distribution and evolution of von Willebrand/integrin A domains: widely dispersed domains with roles in cell adhesion and elsewhere.” Mol Biol Cell 13(10): 3369-87.

### Advantages of the Invention

The present invention provides an improved method for targeting skin care agents and cosmeceuticals, to cellular targets, as well as compositions for such targeting. The method and compositions can be employed for targeting of a wide range of skin care agents or cosmeceuticals and does not depend critically on chemical reactivity or physical properties of the skin care agents to be targeted. By targeting to collagen molecules, methods and compositions according to the present invention provide a more efficient way of targeting that will reduce delivery of the skin care agents or cosmeceuticals to undesired sites, reduce the quantity of skin care agents or cosmeceuticals required, and reduce the frequency and severity of adverse reactions associated with the delivery of an excessive quantity of skin care agent or cosmeceutical or with the delivery of a skin care agent or cosmeceutical to an area where treatment is not needed or is not optimal.

Compositions according to the present invention possess industrial applicability as compositions useful for cosmetic, cosmeceutical, or esthetic purposes. Methods as described herein also include methods for preparation of a medicament for treatments to carry out cosmetic, cosmeceutical, or esthetic functions.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

The transitional phrase “comprising,” as used herein in the specification and claims, also encompasses the transitional phrases “consisting essentially of” and “consisting of” unless “consisting essentially of” or “consisting of” are specifically excluded from the scope therefrom.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, and literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Moreover, as used herein, the transitional phrase “comprising” also encompasses the transitional phrases “consisting essentially of” and “consisting of” unless either “consisting essentially of” or “consisting of” are expressly excluded. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

1. A targeting composition comprising:
(a) a skin care agent or an agent that is a cosmeceutical agent;
(b) an intermediate release linker bound to the skin care agent or cosmeceutical agent;
(c) a targeting moiety bound to the intermediate release linker, the targeting moiety for binding the targeting composition to native collagen fibers; and
(d) optionally, a carrier component to enhance delivery to the skin.

2.-4. (canceled)

5. The targeting composition of claim 1 wherein the targeting moiety is selected from the group consisting of: (i) Trp-Arg-Glu-Pro-Ser-Phe-Met-Ala-Leu-Ser (WREPSFMALS) (SEQ ID NO: 1); (ii) Trp-Arg-Glu-Pro-Ser-Phe-Cys-Ala-Leu-Ser (WREPSFCALS) (SEQ ID NO: 2); and (iii) peptides related to (i) or (ii) by one or more conservative amino acid substitutions.

6. The targeting composition of claim 5 wherein the targeting moiety is a peptide related to SEQ ID NO: 1 or SEQ ID NO: 2 by one or more conservative amino acid substitutions, and wherein the peptide is selected from the group consisting of: Trp-Arg-Asp-Pro-Ser-Phe-Met-Ala-Leu-Ser (WRDPSFMALS) (SEQ ID NO: 3); Trp-Arg-Asp-Pro-Ser-Phe-Cys-Ala-Leu-Ser (WRDPSFCALS) (SEQ ID NO: 4); Trp-Arg-Glu-Pro-Ser-Phe-Met-Ala-Ile-Ser (WREPSFMAIS) (SEQ ID NO: 5); Trp-Arg-Glu-Pro-Ser-Phe-Cys-Ala-Ile-Ser (WREPSFCAIS) (SEQ ID NO: 6); Trp-Arg-Asp-Pro-Ser-Phe-Met-Ala-Ile-Ser (WRDPSFMAIS) (SEQ ID NO: 7); Trp-Arg-Asp-Pro-Ser-Phe-Cys-Ala-Ile-Ser (WRDPSFCAIS) (SEQ ID NO: 8); Gly-Pro-Pro-Gly-Trp-Arg-Glu-Pro-Ser-Phe-Met-Ala-Leu-Ser-Gly-Pro-Pro-Gly (GPPGWREPSFMALSGPPG) (SEQ ID NO: 9); Gly-Pro-Pro-Gly-Trp-Arg-Glu-Pro-Ser-Phe-Cy s-Ala-Leu-Ser-Gly-Pro-Pro-Gly (GPPGWREPSFCALSGPPG) (SEQ ID NO: 10); Gly-Pro-Pro-Gly-Trp-Arg-Asp-Pro-Ser-Phe-Met-Ala-Leu-Ser-Gly-Pro-Pro-Gly (GPPGWRDPSFMALSGPPG) (SEQ ID NO: 11); Gly-Pro-Pro-Gly-Trp-Arg-Asp-Pro-Ser-Phe-Cys-Ala-Leu-Ser-Gly-Pro-Pro-Gly (GPPGWRDPSFCALSGPPG) (SEQ ID NO: 12); Gly-Pro-Pro-Gly-Trp-Arg-Glu-Pro-Ser-Phe-Met-Ala-Ile-Ser-Gly-Pro-Pro-Gly (GPPGWREPSFMAISGPPG) (SEQ ID NO: 13); Gly-Pro-Pro-Gly-Trp-Arg-Glu-Pro-Ser-Phe-Cys-Ala-Ile-Ser-Gly-Pro-Pro-Gly (GPPGWREPSFCAISGPPG) (SEQ ID NO: 14); Gly-Pro-Pro-Gly-Trp-Arg-Asp-Pro-Ser-Phe-Met-Ala-Ile-Ser-Gly-Pro-Pro-Gly (GPPGWRDPSFMAISGPPG) (SEQ ID NO: 15); and Gly-Pro-Pro-Gly-Trp-Arg-Asp-Pro-Ser-Phe-Cys-Ala-Ile-Ser-Gly-Pro-Pro-Gly (GPPGWRDPSFCAISGPPG) (SEQ ID NO: 16).

7. (canceled)

8. The targeting composition of claim 1 wherein the targeting moiety is an elongated peptide structure of Formula (I):
(I)
(SEQ ID NO: 97)
[Gly-Pro-Pro-Gly-X₁-Gly-Pro-Pro-Gly-X₂-Gly-Pro-
Pro-Gly]ₙ
wherein: (1) X₁ and X₂ are one of peptide sequences SEQ ID NO: 1 through SEQ ID NO: 16; and (2) n is an integer from 1 to 15.

9.-11. (canceled)

12. The targeting composition of claim 1 wherein the targeting moiety is a collagen binding site of a platelet collagen binding receptor.

13.-15. (canceled)

16. The targeting composition of claim 1 wherein the targeting moiety of the composition includes a flanking sequence that mimics a sequence found in native collagen or in native elastin.

17.-27. (canceled)

28. The targeting composition of claim 1 wherein the targeting moiety includes the amino acid sequence GVMGFO (SEQ ID NO. 17).

29. The targeting composition of claim 1 wherein the targeting moiety includes a CBD from discoidin domain receptor DDR1 or from discoidin domain receptor DDR2.

30.-31. (canceled)

32. The targeting composition of claim 1 wherein the targeting moiety includes the amino acid sequence GTPGPGGIAGQRGVV (SEQ ID NO: 19).

33.-34. (canceled)

35. The targeting composition of claim 1 wherein the intermediate release linker is stabilized by crosslinking.

36.-39. (canceled)

40. The targeting composition of claim 1 further comprising a cell-penetrating peptide.

41.-46. (canceled)

47. The targeting composition of claim 1 further comprising a transcription-activating peptide.

48. (canceled)

49. The targeting composition of claim 1 wherein the intermediate release linker is a polymer that shields the therapeutic agent of the composition from clearance by macrophages.

50.-61. (canceled)

62. The targeting composition of claim 1 wherein the intermediate release linker does not interact with the skin care active agent or cosmeceutical and does not bind to or otherwise interact with the targeting moiety.

63. The targeting composition of claim 62 wherein the intermediate release linker is a non-protein polymer selected from the group consisting of polyethylene glycol and polypropylene glycol.

64.-68. (canceled)

69. The targeting composition of claim 1 wherein the intermediate release linker is selected from the group consisting of a thiol-containing amino acid sequence derived from keratin or a biosynthesized thiol-containing amino acid sequence mimicking the properties of the thiol-containing amino acid sequence derived from keratin and a hydrophobic amino acid sequence derived from elastin or a biosynthesized hydrophobic amino acid sequence mimicking the properties of the hydrophobic amino acid sequence derived from elastin.

70. (canceled)

71. The targeting composition of claim 1 wherein the linkage between the skin care agent or cosmeceutical and the intermediate release linker is a covalent linkage.

72.-73. (canceled)

74. The targeting composition of claim 1 wherein the linkage between the skin care agent or cosmeceutical and the intermediate release linker is a non-covalent linkage.

75.-76. (canceled)

77. The targeting composition of claim 1 wherein the linkage between the intermediate release linker and the targeting moiety is a covalent linkage.

78.-79. (canceled)

80. The targeting composition of claim 1 wherein the linkage between the intermediate release linker and the targeting moiety is a non-covalent linkage.

81.-82. (canceled)

83. The targeting composition of claim 1 wherein the skin care agent or cosmeceutical is a skin care agent selected from the group consisting of retinoids, hydroxyacids, esters of hydroxyacids, skin treatment products, and Wnt pathway modulators.

84.-158. (canceled)

159. The targeting composition of claim 1 wherein the skin care agent or cosmeceutical is a cosmeceutical selected from the group consisting of a botanical extract from oil palm vegetation liquor; GM-CSF; a nucleic acid expressing GM-CSF; a suspension of a powder of an aliphatic polyester copolymer, a cross-linked silicone elastomer, and at least one hydrolysate or acylated short-chain peptide; a mixture of refined, bleached, deodorized palm oils and red palm olein; a dipeptide incorporating a selenoamino acid; a 3,6-dihydro-2H-pyran; calcium chloride, magnesium chloride, and potassium bromide for restoration of skin barrier function; a composition including nordihydroguiaretic acid, niacinimide, and, optionally, an antioxidant a peptide modified with a triterpenoid; 5-aminolevulinic acid; 3,5-dimethoxy-4′-hydroxystilbene; an alkanediol selected from the group consisting of 1,2-propanediol, butyleneglycol, 2-ethyl-1,3-hexanediol, and 2-methyl-2,4-pentanediol; an ether diol; a diether alcohol; a composition including hyaluronic acid, kokic acid, and glycolic acid; artemetin; hydroquinone or a derivative thereof; an anti-acne agent selected from the group consisting of N-acetylcysteine, adapalene, azelaic acid, benzoyl peroxide, cholate, clindamycin, deoxycholate, erythromycin, flavonoids, glycolic acid, meclocycline, mupirocin, octopirox, phenoxyethanol, phenoxypropanol, pyruvic acid, resorcinol, retinoic acid, salicylic acid, scymnol sulfate, sulfacetamide-sulfur, sulfur, tazarotene, tetracycline, and tretinoin triclosan; melatonin; an anti-psoriatic agent selected from the group consisting of 6-aminonicotinamide, 6-aminonicotinic acid, 2-aminopyrazinamide, anthralin, calcipotriene, 6-carbamoylnicotinamide, 6-chloronicotinamide, 2-carbamoylpyrazinamide, corticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, and trichothecene mycotoxins; an anti-rosacea agent selected from the group consisting of azelaic acid and metronidazole sulfacetamide; a histamine receptor H₁ antagonist selected from the group consisting of doxepin hydrochloride, carbinoxamine maleate, clemastine fumarate, diphenhydramine hydrochloride, dimenhydrinate, pyrilamine citrate, tripelennamine hydrochloride, tripelennamine citrate, chlorpheniramine mdialeate, brompheniramine maleate, hydroxyzine hydrochloride, hydroxyzine pamoate, cyclizine hydrochloride, cyclizine lactate, meclizine hydrochloride, promethazine hydrochloride, cyproheptadine hydrochloride, phenindamine tartrate, acrivastine, cetirizine hydrochloride, azelastine hydrochloride, levocabastine hydrochloride, loratidine, desloratidine, ebastine, mizolastine, and fexofenadine; a histamine receptor H₂ antagonist selected from the group consisting of cimetidine and ranitidine; a histamine receptor H₃ antagonist a histamine receptor H₄ antagonist a kinin receptor antagonist a leukotriene receptor antagonist vitamin E; vitamin E acetate; tocotrienol; progesterone; capsaicin; capsicum oleoresin; menthol; methyl salicylate; benzophenone-3; octyl methoxycinnamate; benzocaine; and lidocaine.

160.-162. (canceled)

163. The targeting composition of claim 1 further comprising the optional carrier component, wherein the optional carrier component is a pharmaceutically acceptable carrier, diluent, or excipient.

164.-204. (canceled)
